(19) United States
(12) Patent Application Publication
(10) Pub. No.: US 2012/0071338 A1
(43) Pub. Date: Mar. 22, 2012

(54) POLYMERIZATION-BASED AMPLIFICATION FOR IMMUNOSTAINING AND BIODETECTION

(76) Inventors: Christopher Bowman, Boulder, CO (US); Vivek Balasubramaniam, Superior, CO (US); Bradley Berron, Lexington, KY (US); Heather Avens, Lafayette, CO (US); Gregory Seedorf, Denver, CO (US)

(21) Appl. No.: 13/183,919

(22) Filed: Jul. 15, 2011

Related U.S. Application Data
(60) Provisional application No. 61/364,689, filed on Jul. 15, 2010.

(51) Int. Cl. C40B 30/04; B82Y 5/00

(52) U.S. Cl. 506/9; 977/773

(57) ABSTRACT

The invention provides polymerization-based signal amplification methods in which labeling moieties are incorporated into a polymer mass or film formed at a biorecognition site. When the labeling moieties are fluorescent, the invention can be used to provide an immunofluorescent staining method which is non-enzymatic. The invention also provides kits useful for the immunofluorescent staining methods of the invention.

### CROSS-REFERENCE TO RELATED APPLICATIONS

This application claims the benefit of U.S. Provisional Application No. 61/364,689, filed Jul. 15, 2010, which is hereby incorporated by reference in its entirety to the extent not inconsistent with the disclosure herein.

### ACKNOWLEDGEMENT OF GOVERNMENT SUPPORT

This invention was made with government support under grant number R21 CA127884 awarded by the National Institutes of Health. The government has certain rights in the invention.

### BACKGROUND

Immunostaining may be defined as the staining of a specific substance by using an antibody against it which is complexed with a staining medium. Immunofluorescent staining of cells is a central technology in cell biology that is invaluable for localization of cellular proteins, elucidating cellular functions and aiding in pathology-based disease diagnosis. To localize fluorescent signal at antigenic biorecognition sites, an organic fluorophore is typically coupled to the primary or secondary antibody. In a similar approach, a biotinylated antibody probe is used, followed by fluorescently labeled avidin which specifically binds biotin.

Though being straightforward to implement, direct labeling of protein probes has limitations for detection of low abundance antigens (Van Heusden, 1997, J. Histochem. Cytochem. 45, 315-319). Enzymatic fluorescent signal amplification techniques which utilize an enzyme to deposit fluors near biorecognition sites enable far more sensitive detection and are commercially available, yet they also have limitations.

One popular enzymatic approach is tyramide signal amplification (TSA), also called catalyzed reporter deposition (CARD), in which probes are coupled to horseradish peroxidase (HRP) which catalyzes the formation of short-lived fluorescently-labeled tyramide radicals that rapidly react with chemical groups near the enzyme, thereby immobilizing the fluorophores. Though enabling highly sensitive detection, TSA is often hindered by loss of signal localization as the tyramide radicals diffuse away from the biorecognition site prior to immobilization (Kersterns 1995, J. Histochem. Cytochem. 43, 347-352; Wiedorn 1999, Histochem. Cell Biol. 111, 89-95). Additionally, non-specific staining from endogenous cellular peroxidases is often difficult or impractical to eliminate completely (Hunyady 1996 Histochem. Cell Biol. 106, 447-456; Ishii 2004 FEMS Microbiol. Ecol. 50, 203-212; Pavlekovic 2009 J. Microbiol. Methods 78, 119-126).

Another enzymatic method, enzyme-linked fluorescence (ELF) generates a precipitating fluorescent substrate but is only available with only a single fluorophore. (Larison, 1995 J. Histochem. Cytochem. 43, 77-83)

Alternatively to enzymatic approaches, probe-functionalized quantum dots (QDs) have also been employed for cellular immunostaining, (Wu, 2003, Nat. Biotechnol. 21, 41-46) yielding an exceptionally photostable stain and providing especially narrow emission spectra, reducing cross-talk in multiplexed staining of multiple antigens. Though QD are brighter than organic fluorophores, they typically do not achieve the same increase in fluorescence intensity as enzyme-based methods. (Wu 2003, Nat Biotechnol 21:41-46; Speel 1999 J. Histochem. Cytochem. 47, 281-288)

Methods for amplification of signals from antibody microarrays include use of fluorescent nanoparticles (NPs) (Wiese et al, 2003, Luminescence, 18: 25-30 Jarras et al., 2008, Proteome Res. 7:1308-14), fluorescent proteins (Haab et al, 2003, Proteomics, 3: 2116-22; Nielson, 2004, J. Immol, Methods, 290: 107-20), use of quantum dots (Geho et al., 2005, Bioconjug. Chem., 16: 559-66); and enzymatic techniques such as tyranamide signal amplification and rolling circle amplification (Nielson, 2004, J. Immol, Methods, 290: 107-20).

Polymerization-based methods of biorecognition events involving antibodies have also been reported. U.S. Pat. No. 7,354,706 to Rowlen et al. reports use of photopolymerization for amplification and detection of a molecular recognition event. WO/2007/095464 to Kuck reports signal amplification of biorecognition events using photopolymerization in the presence of air. Additionally, the incorporation of fluorescent NPs into films generated by polymerization-based signal amplification (PBA) with a conjugate of streptavidin (SA) and eosin has been shown in an antibody microarray format to yield a 100-fold improvement in sensitivity compared to the use of fluor-labeled streptavidin (Avens, 2009, Acta Biomater 6: 83-89).

Application of polymerization-based methods for detection of various types of molecular recognition events has been reported. Sikes et al.(H. D. Sikes et al., Nat. Mater. 7 (2008) 52-56) reported a visual detection method utilizing photopolymerization for signal amplification on a commercially available biodetection platform (Jenison et al., Nature Biotechnology 2001, 19, 62-65; Jenison, Ret al., Clinical Chemistry 2001, 47, 1894-1900). A more recent approach to signal amplification involves polymerization-based amplification (PBA) systems capable of direct visualization and detection of minute levels of biotinylated biomolecules present on glass slides (R. Hansen et al., 2008, Biomacromolecules 9, 355-362; R. R. Hansen, et al., 2008, Anal. Bioanal. Chem. 392 167-175). These systems employed streptavidin (SA) labeled photoinitiators capable of both recognizing surface-bound biotin and eliciting the formation of macroscale polymeric material upon surface-initiated polymerization reactions. Hansen (2008, Anal Bioanal Chem 392:167-175) also describes incorporation of fluorescent NPs into PBA films generated by SA-eosin. Other references related to polymerization-based signal amplification in a variety of configurations include Sikes (2009, Lab on a chip 9(5):653-6); Lou (2005, Anal. Chem. 77, 4698-4705), and Hansen (Hansen, 2009, Anal. Biochem. 386, 285-287).

Surface initiated polymerization from surface confined initiators has also been reported. Biesalski et al. report poly(methyl methacrylate) brushes grown in situ by free radical polymerization from an azo-initiator monolayer covalently bound to the surface (Biesalski, M. et al., (1999), J. Chem. Phys., 111(15), 7029). Surface initiated polymerization for amplification of patterned self-assembled monolayers by surface-initiated ring opening polymerization (Husemann, M. et al., Agnewandte Chemie Int. Ed. (1999), 38(5) 647-649) and atom transfer radical polymerization (Shah, R. R. et al., (2000), Macromolecules, 33, 597-605) has been also reported.

### SUMMARY

In one aspect, the invention provides methods to detect molecular recognition events. In an embodiment, the methods of the invention are based on polymerization-based amplification of the signal due to each molecular recognition event. In an embodiment, labeling moieties are incorporated into the polymer, so that the amplification may be defined in terms of the signal obtained from the labeling moieties. In one embodiment, the invention provides a fluorescent polymerization-based signal amplification method for immunofluorescent staining of cells. In another embodiment, the invention provides a fluorescent polymerization-based signal amplification method for biodetection in an array format.

In an embodiment of the methods of the invention, a photoinitiator molecule may be coupled to a probe which can interact with a target at a biorecognition site. Subsequent light exposure can then initiate the conversion of monomer in the vicinity of the biorecognition site to form a polymer mass or film localized at the biorecognition site. If the photoinitiator is a radical photoinitiator, the amplification inherent in radical polymerization reactions can be harnessed. The incorporation of labeling moieties into the polymer mass or film can increase the number of labeling moieties associated with a particular biorecognition site as compared to methods in which one labeling moiety is directly coupled to each probe.

In an embodiment, the labeling moieties can be detectable particles. For example, the labeling moieties may be fluorescent, magnetic, radioactive, electrically conducting, absorptive/colored, MRI contrast agents, or electron microscopy contrast agents (e.g. electron opaque particles such as metal particles). In an embodiment, the detectable particles are selected together with the monomer solution in order to enhance incorporation of the detectable particles into the polymer mass or film.

In an embodiment, the incorporation of fluorescent particles in the polymer mass leads to an overall gain in fluorescence signal at the biorecognition site as compared to methods in which one labeling moiety is directly coupled to each probe. In an embodiment, the gain can be quantified as the ratio of the amount of fluorescence resulting from incorporation of fluorescent nanoparticles into the polymer mass to the amount of fluorescence resulting from a fluorescent labeling moiety being directly coupled to each probe. In different embodiments, this ratio may be at least 10, 20, 30, 40, 50, 60, 70, 80, 90, 100, 150, 200, 250, 300, 350, 400, 450, or 500. This gain may be coupled to a particular polymer film thickness range. In different embodiments, the polymer film thickness may be from 1 nm to 1 micrometer, from 5 nm to 500 nm, or from 5 nm to 200 nm. In an embodiment, the fluorescence can be detected with epifluorescence microscopy.

In an embodiment, the invention provides a polymerization-based signal amplification method for immunofluorescent staining of cells that combines the attributes of high sensitivity, good signal localization, photostable fluorescence, and multiplexed staining ability. The immunofluorescent staining methods of the invention can provide a non-enzymatic, polymerization-based signal amplification method without interference from endogenous enzymes or the need for any enzymatic process. In this embodiment, photoinitiator molecules may be coupled to protein probes such that light exposure initiates the conversion of monomer and fluorescent moieties into a highly fluorescent polymer film localized at the biorecognition site. In an embodiment, the protein probe is an antibody. The antibody concentration required for detection of the target may be significantly less than in typical immunofluorescent protocols. In different embodiments, the antibody concentration may be at least one order of magnitude less, at least two orders of magnitude less, or at least three orders of magnitude less than in typical immunofluorescent protocols (e.g. a protocol in which a fluorophore such as streptavidin-Alexa Fluor 488 is attached to a biotin binding molecule). Therefore, this approach can reduce antibody consumption during staining as compared to typical immunofluorescent protocols.

In an embodiment, the staining methods of the invention detect a molecular recognition event between an antibody and a target of a cell. The antibody which undergoes molecular recognition with the target may be a primary antibody. The target may be a target protein, which may be an antigen containing at least one epitope capable of binding to the antibody. The epitope may be located inside the cell membrane, outside the cell membrane, or at the cell membrane. For epitopes located within the cell membrane, the epitope may be located inside or outside any other membrane within the cell such as the nuclear membrane or membranes around other organelles. In addition to detection of proteins, antibodies are also able to achieve specific detection of a wide variety of small molecules (e.g. haptens); in other embodiments the target may be such a small molecule.

In an embodiment, the invention provides a method of detecting a molecular recognition event between an antibody and a target protein of a cell attached to a substrate, the method comprising the steps of: treating the cell with a blocking agent; contacting the antibody with the target protein under conditions effective to form an antibody-target protein complex; removing antibody not complexed with the target protein; labeling the antibody-target protein complex with a photoinitiator label wherein the photoinitiator label comprises a photoinitiator; removing photoinitiator label not attached to the antibody-target protein complex; contacting the photoinitiator-labeled antibody-target protein complex with a polymer precursor solution comprising a water soluble monomer, an optional co-initiator and plurality of fluorescent nanoparticles, wherein the average size of the fluorescent nanoparticles is from 10 to 50 nm; exposing the photoinitiator-labeled antibody-target protein complex and the polymer precursor solution to light, thereby forming a polymer gel attached to the cell and incorporating at least some of the fluorescent nanoparticles from the polymer precursor solution; removing unpolymerized polymer precursor and fluorescent nanoparticles not incorporated into the polymer gel; and detecting fluorescence from the nanoparticles in the polymer gel, thereby detecting the molecular recognition event.

In an embodiment, the fluorescent nanoparticles are selected to limited nonspecific binding or interaction in a biological environment such as within a cell or assembly of cells, within a tissue or in a cell suspension. Such biological environments may present greater nonspecific binding challenges than microarray environments. The cells of interest may be cultured cells; the cultured cells may adherent cells. In an embodiment, the extent of nonspecific binding may be indicated by comparison of the fluorescence signal from an area of interest to the signal away from the area of interest (background signal). In an embodiment, the ratio of the fluorescence signal per unit area from nanoparticles in the polymer gel to a background fluorescence signal per unit area is greater than 5. In other embodiment, the signal to noise ratio may be greater than 3. The unit area may be a pixel of a detector or an image produced by a detector. The fluorescence signal may be an average of the signal from a plurality of pixels.

The photoinitiator included in the photoinitiator label may be a photocleavable unimolecular initiator or a bimolecular photoinitiator. If the photoinitiator label comprises a bimolecular photoinitiator, a co-initiating molecule is typically included in the polymer precursor solution. The photoinitiator label may comprise a photoreducible dye, with an amine co-initiating molecule being included in the polymer precursor solution and the photoinitiator label and the polymer precursor solution being exposed to visible light in order to initiate the polymerization process. The polymer precursor solution may be treated to reduce the amount of oxygen dissolved therein, thereby minimizing oxygen inhibition of the polymerization process.

When the target protein is located within the interior of the cell, the methods of the invention will generally include the step of subjecting the cell to a fixation step. In order to facilitate access of the antibody to the target protein, the cell may be subjected to a permeabilizing treatment prior to contacting the antibody with the target protein.

In another embodiment, the staining methods of the invention can be used to detect a molecular recognition event between an antibody and target which is a cellular macromolecule. It is known in the art that antibodies are capable of achieving sensitive detection of cellular macromolecules such as nucleic acids, carbohydrates and lipids. In other embodiment, the methods of the invention can be used for detection of any target chemical species in the cell for which a primary antibody has been developed.

In an embodiment, the invention provides a method of detecting a molecular recognition event between an antibody and a target chemical species of a cell attached to a substrate, the method comprising the steps of: treating the cell with a blocking agent; contacting the antibody with the target under conditions effective to form an antibody-target complex; removing antibody not complexed with the target; labeling the antibody-target complex with a photoinitiator label wherein the photoinitiator label comprises a photoinitiator; removing photoinitiator label not attached to the antibody-target complex; contacting the photoinitiator-labeled antibody-target complex with a polymer precursor solution comprising a water soluble monomer, an optional co-initiator and plurality of fluorescent nanoparticles, wherein the average size of the fluorescent particles is from 10 to 50 nm; exposing the photoinitiator-labeled antibody-target complex and the polymer precursor solution to visible light, thereby forming a polymer gel attached to the cell and least some of the fluorescent nanoparticles from the polymer precursor solution; removing unpolymerized polymer precursor and fluorescent nanoparticles not incorporated into the polymer gel; and detecting fluorescence from the nanoparticles in the polymer gel, thereby detecting the molecular recognition event.

In an embodiment, the cell comprises a plurality of target species and a plurality of antibodies are contacted with the cell, forming a plurality of antibody-target complexes and forming polymer gel at a plurality of antibody-target complexes. The polymer gel may form a layer connected to a plurality of antibody-target complexes. The dimensions of the polymer film or mass generated during polymerization-based amplification can affect the resolution of the stain. In an embodiment, polymerization is controlled so that the thickness of the film is less than the minimum characteristic dimension of the feature to be imaged. In different embodiments, the thickness of the film is from 1 nm to 5 micrometers, from 1 nm to 2 micrometers, from 1 nm to 1 micrometer, less than one micrometer, from 5 nm to 500 nm, or from 5 nm to 200 nm. Monomers useful for control of film thickness (less than 1 micrometer) include, but are not limited to, poly(ethylene glycol) diacrylate (PEGDA) and mixtures of poly(ethylene glycol) acrylate (PEGA) and poly(ethylene glycol) diacrylate (PEGDA).

In other embodiments, growth of a relatively thick film may be desirable. For example, sufficiently thick films can be seen with the unaided eye. In an embodiment, the film thickness may be greater than 1 micrometer. Film thicknesses greater than micrometer can be useful for detection of targets on or external to the cell membrane. Monomers useful for formation of films thick enough to be seen with the unaided eye include, but are not limited to mixtures of mono and difunctional modified and/or unmodified acrylamide monomers.

FIG. 1 is a conceptual depiction of fluorescent polymerization-based amplification (FPBA) for immunostaining of a nuclear envelope membrane protein. Primary antibody (light grey Y-shape) binds its target protein (rectangles outlined in black), and a biotinylated secondary antibody probe (dark grey Y-shape) binds primary antibody. Streptavidin (cross-shape) coupled to eosin photoinitiators (medium grey) binds to biotin on the secondary antibody (dark grey Y-shape). Divinyl monomer, coinitiator, and fluorescent nanoparticles (NPs) are applied and the sample is exposed to visible light to initiate polymerization. The growing polymer film entraps the fluorescent NPs, anchoring them to the biorecognition site.

In another embodiment, sequential polymerization steps, each incorporating a different labeling moiety, can be used to enable detection of a plurality of antigens. For example, each step may use fluorescent nanoparticles with a different emission wavelength (color).

In another aspect, the methods of the invention can be used to detect molecular interaction between a receptor and a ligand in a cell. In such a method, a photoinitiator-labeled receptor-ligand complex is formed. In different embodiments, the photoinitiator may be linked to the receptor or the ligand. In this case, the method may include the steps of forming a photoinitiator labeled ligand-receptor complex, contacting this complex with a polymer precursor solution including a plurality of labeling moieties, exposing the complex and the precursor solution to light, thereby forming a polymer incorporating a plurality of labeling moieties, and detecting the labeling moieties, thereby detecting the molecular recognition event. In an embodiment, the polymer mass is bound to or localized near the ligand receptor complex, thereby forming a polymer label.

The cells to be stained using the methods of the invention may be in solution or attached to a solid support. For cells in solution, the methods of the invention may be used to prepare polymer-labeled cells for flow cytometry, fluorescence activated cell sorting or magnetic activated cell sorting. It is also possible to specifically form polymer on the exterior of specific cells to sort based on cell size. For cells on a solid support, the methods of the invention can be used to prepare polymer-labeled cells for a variety of immunostaining analysis techniques known to the art, including fluorescence microscopy or confocal microscopy (for fluorescent labels) or radiography (for radioactive labels). In an embodiment, the invention provides kits for performing the polymerization-based amplification methods of the invention. In an embodiment, a kit suitable for use with a biotin-labeled target protein-antibody complex comprises a first solution including a conjugate of a photoinitiator and a biotin binding protein, a second solution including a monomer, and a plurality of labeling moieties. In another embodiment, a kit suitable for use with a target protein-primary antibody complex comprises a first solution including conjugate of a secondary antibody and a photoinitiator, a second solution including a monomer, and a plurality of labeling moieties.

For biodetection in microarrays, the detection and amplification scheme can be used to detect and amplify a variety of molecular recognition interactions. In different embodiment, the molecular interaction reaction may be between nucleic acids, an antibody and an antigen, or a first and a second protein. In this aspect of the invention, the surface-bound moiety may be referred to as the probe, and the photoinitiator coupled most directly to the target. Monomers useful for biodetection in microarrays include, but are not limited to mixtures of acrylamide and bisacrylamide, poly(ethylene glycol) diacrylate (PEGDA) and mixtures of poly(ethylene glycol) acrylate (PEGA) and poly(ethylene glycol) diacrylate (PEGDA). In an embodiment, the fluorescent polymerization-based methods of the invention are capable of detecting a quantity of surface-bound target molecules 40 zeptomole or greater.

### BRIEF DESCRIPTION OF THE FIGURES

FIG. 1: Conceptual depiction of FPBA for immunostaining of a nuclear envelope membrane protein. Primary antibody (light grey) binds its target protein rectangles), and a biotinylated secondary antibody probe (dark grey) binds primary antibody. Streptavidin coupled to eosin photoinitiators (medium grey) binds to biotin on the secondary antibody. Divinyl monomer, coinitiator, and fluorescent NPs are applied and the sample is exposed to visible light to initiate polymerization. The growing polymer film entraps the fluorescent NPs, anchoring them to the biorecognition site and enabling detection.

FIGS. 2A-2F: Comparison of FPBA vs. SA-Alexa488 for immunofluorescent imaging of various antigens. (A) Human endothelial cells were immunostained with mouse anti-vimentin (1:50,000), biotinylated anti-mouse secondary (1:400), and fluorescently labeled by FPBA. (B) Human endothelial cells were stained for vWF with rabbit anti-vWF (1:250,000), biotinylated anti-rabbit secondary (1:500), and fluorescently labeled by FPBA. (C) Human endothelial cells were immunostained with mouse anti-NPC (1:1,000), biotinylated anti-mouse secondary (1:400) and fluorescently labeled by FPBA. (D,E,F) Same as (A, B, C) respectively, except that the fluorescent labeling is achieved using SA-Alexa488 rather than FPBA. Scale bar is 5 μm. Images were taken on a confocal laser scanning microscope (CLSM) using a 40× oil objective for A,B,D, and E, and a 63× oil objective for C, and F. The nucleus is counterstained with DAPI. DAPI staining in C and F confirmed that immunostaining is localized around the nucleus, but the DAPI stain is not depicted so as to more clearly indicate the NPC staining.

FIGS. 3A ((i)-(iv)), 3B ((i)-(iv)), and 3C ((i)-(ii)): Comparison of FPBA to other detection methods for the fluorescent imaging of NPC in human fibroblast cells. FPBA (A), is compared to TSA (B), and SA-Alexa488 (C). Cells were stained using 1:10² (i), 1:10³ (ii), 1:10⁴ (iii), or 1:10⁵ (iv) dilution of primary antibody. In all cases, the secondary antibody was biotinylated anti-mouse at a 1:400 dilution. These images were obtained by epifluorescence microscopy, using a 20× objective lens, a four second exposure time and no binning of pixels. The scale bar is 200 μm. DAPI nuclear counter-staining verified that staining is centered around the nuclei, but the DAPI stain is not depicted so as to more clearly indicate the NPC staining.

FIGS. 4A-4B: A) Comparison of fluorescence intensity of NPC staining in human fibroblast cells utilizing FPBA (light gray), TSA (dark gray), or SA-Alexa488 (black). The p-values for a one-tailed student's t-test between FPBA and TSA were p₁=1.1×10⁻³, p₂=3.4×10⁻², p₃=2.7×10⁻¹¹, p₄=1.1×10⁻³, and p₅=1.9×10⁻¹. Although p-values were determined for all data sets, it is of note that the data sets for the no primary antibody condition were not normally distributed, nor were the FPBA data for the 1:100 antibody dilution. *Indicates that most of these signal intensities were saturated. **Indicates that these measurements were not taken. (B) Nonspecific staining (“noise”) was assessed in each cell by measuring the fluorescence in an area equal to the size of the nucleus, but immediately adjacent to the nucleus. For each cell, the intensity of staining in the nucleus (“signal”) was divided by the noise value. The ratio of signal/noise is compared between FPBA (light gray) and TSA (dark gray). Although these data sets were not normally distributed, p-values for a one-tailed t-test were determined: p₁=8.5×10⁻², p₂=6.0×10⁻², p₃=1.0×10⁻⁴, p₄=3.6×10⁻³. For both A and B, measurements were taken for human fibroblast cells stained under the same staining and imaging conditions as utilized for the images in FIG. 3. Also, for each primary antibody dilution in A and B, measurements were made for all cells in each of four images obtained from at least two separate staining sessions; however, when it was not possible to take a noise measurement immediately adjacent to the nucleus, that cell was not included in the signal/noise measurements. For A and B, intercellular background signal measured far from the cells was subtracted from the measured signal and noise intensities. DAPI staining was used to delineate areas that are expected to be stained positive for NPC.

FIGS. 5A-5C: Dual color immunostaining of two antigens is achieved by sequential FPBA reactions. (a) Human endothelial cells are immunostained for NPC using Nile red NPs (fluorescence in oval central region), followed by a second round of immunostaining for vimentin using yellow/green NPs (fluorescence in outer region). (b) As a negative control for NPC staining, the NPC primary antibody was omitted from the antibody dilution buffer during the first staining reaction. (c) As a negative control for vimentin staining, the vimentin primary antibody was omitted from the antibody dilution buffer during the second staining reaction. The nucleus is stained with DAPI (fluorescence in oval central region). The scale bar is 10 microns. Images were taken with a 40× oil objective on a CLSM. Mouse anti-NPC antibody was used at 1:1,000 dilution, mouse anti-vimentin antibody was used at 1:5,000 dilution, and biotinylated anti-mouse antibody was used at 1:400 dilution.

FIG. 6. FPBA, SA-Alexa488, and SA-FITC were compared for their photostability during imaging. Human fibroblast cells were stained for vimentin and are fluorescently labeled by either FPBA (yellow/green NPs) (solid squares), SA-Alexa488 (open circles), or SA-FITC (open triangles). The cells were continuously illuminated while images were taken at the indicated times after the illumination begins (time=0s). The images were obtained by epifluorescence microscopy, with binning set to 2. Exposure times: FPBA=0.125 sec; SA-Alexa488 and SA-FITC=1 sec. Mouse anti-vimentin antibody was used at 1:500 dilution and biotinylated anti-mouse antibody was used at 1:400 dilution.

FIG. 7: Comparison of the fluorescence emission spectra for 20 nm yellow/green NPs either in water (solid line) or entrapped in a polyacrylamide gel (dotted line). NPs were 0.05 wt %. The monomer formulation was 5.2 M acrylamide, 130 mM bisacrylamide, 210 mM MDEA and 35 mM VP in water. The polymerization was photoinitiated using 500 ng/mL eosin and exposure to 40 mW/cm² light of wavelengths greater than 480 nm.

FIGS. 8A-8C: Introduction of terminology for characterization of fluorescent films generated from a range of eosin photoinitator surface densities. Here, 0.05 wt % 100 nm yellow/green carboxylate-functionalized NPs were used in an acrylamide monomer formulation (5.2 M acrylamide, 130 mM bisacrylamide, 210 mM MDEA and 35 mM VP in water). (a) The term “Overall Gain” is introduced to describe the total increase in film fluorescence seen with an increase in eosin photoinitiator surface density. (b) The term “Fluorescence Gain” is introduced to indicate the increase in the observed fluorescence as a result of an increase in film thickness. (c) The term “Thickness Gain” is introduced to describe the change in film thickness achieved by an increase in eosin photinitiator surface density. Polymerization was initiated by a 30 minute exposure to 40 mW/cm² light at wavelengths greater than 480 nm.

FIGS. 9A-9C: Comparison of the overall gain (a), fluorescence gain (b) and thickness gain (c) measured for polymerizations performed with carboxylate-functionalized NPs of various sizes (0.05 wt %) added to an acrylamide monomer formulation (5.2 M acrylamide, 130 mM bisacrylamide, 210 mM MDEA and 35 mM VP in water). Polymerizations were initiated by a 30 minute exposure to 40 mW/cm² light of wavelengths greater than 480 nm. Films generated in the absence of NPs were not fluorescent. The letters denote groups of conditions that are statistically different (α=0.05)

FIG. 10: Normalized overall gain (solid squares), normalized fluorescence gain (open circles), and normalized thickness gain (open triangles) for polymer films generated by surface mediated initiation with eosin using a PEGDA monomer formulation (420 mM PEGDA, 210 mM MDEA, and 35 mM VP in water) containing carboxylate-functionalized dark red NPs of various sizes (0.05 wt %). Polymerizations were initiated by a 30 minute exposure to 40 mW/cm² light of wavelengths greater than 480 nm. Within each gain type, normalization is performed by dividing each value by the highest gain value. For the overall gain and fluorescence gain, the 20 nm condition is significantly different (α=0.05) than the 40 condition. Also, the thickness gain for the polymerization without NPs (0 nm) is significantly different (α=0.05) from the 20 nm, 40 nm, and 200 nm conditions.

FIG. 11: Normalized overall gain (solid squares), normalized fluorescence gain (open circles), and normalized thickness gain (open triangles) for polymer films generated by surface mediated initiation with eosin using an acrylamide monomer formulation (5.2 M acrylamide, 130 mM bisacrylamide, 210 mM MDEA and 35 mM VP in water) with 0.05 wt % 200 nm yellow/green nanoparticles. Use of carboxy-versus anime-functionalized NPs is compared. Polymerizations were initiated by a 30 minute exposure to 40 mW/cm² light of wavelengths greater than 480 nm. Within each gain type, normalization is performed by dividing each value by the highest gain value. For fluorescence gain and thickness gain, the observed difference in gain between the amine- and carboxy-functionalized surfaces is statistically significant (α=0.05)

FIG. 12: Normalized overall gain (solid squares), normalized fluorescence gain (open circles), and normalized thickness gain (open triangles) for polymer films generated by surface mediated initiation with eosin using different concentrations of bisacrylamide added to 5.2 M acrylamide, 210 mM MDEA, 35 mM VP, and 0.05 wt % 20 nm carboxylate-functionalized yellow/green NPs in water. (2 wt % bisacrylamide is 130 mM.) Polymerizations were initiated by a 30 minute exosure to 40 mW/cm² light of wavelengths greater than 480 nm. Within each gain type, normalization is performed by dividing each value by the highest gain value. Within each gain type, all the values are significantly different (α=0.05)

FIG. 13: Normalized overall gain (solid squares), normalized fluorescence gain (open circles), and normalized thickness gain (open triangles) for polymer films generated by surface mediated initiation with eosin using either an acrylamide monomer formulation (5.2 M acrylamide with 130 mM bisacrylamide) or a PEGDA formulation (420 mM PEGDA), each with 210 mM MDEA, 35 mM VP, and 0.05 wt % 20 nm carboxylate-functionalized Dark red NPs. Polymerizations were initiated by a 30 minute exposure to 40 mW/cm² light of wavelengths greater than 480 nm. For all three types of gain, the observed difference between the acrylamide and PEGDA monomer formulations is statistically significant (α=0.05)

FIG. 14: Schematic of the posited factors influencing NP incorporation into films generated by surface-mediated initiation of polymerization.

FIG. 15: Two different monomer/NP formulations with different anticipated aptitudes for immobilizing fluorescent NPs are compared for their suitability in the fluorescent polymerization-based signal amplification platform for biodetection. Solid squares represent 5.2 M acrylamide, 130 mM bisacrylamide, 210 mM MDEA, 35 mM VP and 0.05 wt % 20 nm yellow/green NPs in water. Open circles represent 620 mM PEGA, 19 mM PEGDA, 210 mM MDEA, 35 mM VP, and 0.05 wt % 100 nm carboxylate-functionalized yellow/green NPs in water. Polymerizations were initiated by a 30 minute exposure to 40 mW/cm² light of wavelengths greater than 480 nm. The limit of detection (S/N>3) for each system is indicated by an asterisk.

FIGS. 16A-16B: Comparison of using an acrylamide (a) versus a PEGDA (b) monomer formulation for FPBA-based staining of the nuclear pore complex of endothelial cells. The acrylamide monomer formulation comprises 5.2 M acrylamide, 130 mM bisacrylamide, 210 mM MDEA, 35 mM VP in water, and 0.05 wt % yellow/green fluorescent NPs in water. The PEGDA monomer formulation comprises 420 mM PEGDA, 210 mM MDEA, 35 mM VP, and 0.05 wt % yellow/green fluorescent NPs in water. Polymerizations were initiated by a 20 minute exposure to 30 mW/cm² light of wavelengths greater than 480 nm. The scale bar indicates 10 μm.

### DETAILED DESCRIPTION

In general, the methods of the invention can be used to generate and amplify a signal due to many types of molecular recognition events that can be described by the following equation:

A+B+In→A−B−In (Eqn. 1)

where A and B are the species of interest that undergo molecular recognition and In is a photoinitiator. A is the probe species and B is the target species. For a microarray, the probe A may be attached to the substrate. The target species, B, and/or the photoinitiator may comprise a linking group which allows selective binding of the photoinitiator to the target or the A-B complex. As an example, the target species may comprise biotin and the initiator avidin. In an embodiment, the target species comprises one of biotin and a biotin-binding protein and the probe species comprises the other of biotin and a biotin-binding protein. Biotin-binding proteins include avidin, streptavidin, and Neutravidin (a deglycosylated form of avidin).

When the initiator comprises a linking group, Equation 1 may also be written as:

A+B+C−In→A−B−C−In (Eqn. 2)

where C comprises an entity which allows selective binding of the photoinitiator to the target or the A-B complex.

Agents capable of participating in molecular recognition events include, but are not limited to, agonists and antagonists for cell membrane receptors, toxins and venoms, viral epitopes, hormones (e.g., opiates, steroids, etc.), hormone receptors, peptides, enzymes, enzyme substrates, substrate analogs, transition state analogs, cofactors, drugs, proteins, and antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. In different embodiments, the detection and amplification scheme can be used to detect and amplify the molecular recognition interaction between nucleic acids, an antibody and an antigen, and a first and a second protein. As used herein, molecular recognition interactions are those in which the probe recognizes and selectively binds a target, resulting in a target-probe complex. Molecular recognition interactions also involve the formation of noncovalent bonds between the two species. The binding occurs between specific regions of atoms (molecular domains) on the probe species which have the characteristic of binding or attaching specifically to unique molecular domains on specific target species. Molecular recognition interactions can also involve responsiveness of one species to another based on the reciprocal fit of a portion of their molecular shapes.

The target and probe are two species of interest which undergo molecular recognition. The target may also be referred to as a ligand. The probe may also be referred to as a receptor. In an embodiment, at least some characteristics of the probe are known. In an embodiment, the probe is an oligonucleotide whose sequence is known or partially known. In other embodiment, the sequence of the probe may not be known, but it is known to be complementary to a possible target species. Typically, the probe will be selected so that it is capable of selected recognition with the known or suspected identity of the target. In some cases a single probe can be used to detect the presence of a target. In other cases more than one probe will be necessary to detect the presence of or identify a target.

In order for molecular interaction between the target and the probe to identify the target, the molecular interaction between the target and the probe must be sufficiently specific. For hybridization, the selectivity is a measure of the specificity of the molecular recognition event. “Selectivity” or “hybridization selectivity” is the ratio of the amount of hybridization (i.e., number of second nucleic acids hybridized) of fully complementary hybrids to partially complementary hybrids, based on the relative thermodynamic stability of the two complexes. For the purpose of this definition it is presumed that this ratio is reflected as an ensemble average of individual molecular binding events. Selectivity is typically expressed as the ratio of the amount of hybridization of fully complementary hybrids to hybrids having one base pair mismatches in sequence. Selectivity is a function of many variables, including, but not limited to: temperature, ionic strength, pH, immobilization density, nucleic acid length, the chemical nature of the substrate surface and the presence of polyelectrolytes and/or other oligomers immobilized on the substrate or otherwise associated with the immobilized film.

In an embodiment, the molecular recognition event occurs between a target and a probe to form a target-probe complex. The target-probe complex is labeled with a photoinitiator label which comprises a photoinitiator. In one embodiment, the photoinitiator is capable of being activated by ultraviolet (UV) light and photopolymerization is initiated by exposure to a source of UV light. In this embodiment, a co-initiator may not be required. In another embodiment, the photoinitiator is capable of being activated by visible light and photopolymerization is initiated by exposure to a source of visible light. In an embodiment, the photoinitiator is part of a two-part photoinitiator system comprising a photoinitiator and a co-initiator. In an embodiment, the photoinitiator interacts with the co-initiator to generate free-radicals upon exposure to a source of visible light.

The amplification scheme relies on the large number of propagation events that occur for each initiation event. Depending on the specific polymerization system used (light intensity, initiator concentration, monomer formulation, temperature, etc.); each initiator can lead to the polymerization of as many as 10²-10⁶ monomer units. Thus, each single molecular recognition event has the opportunity to be amplified by the polymerization of up to 10⁶ or 10⁷ monomers, each of which may be fluorescent or enable detection of its presence through one of a variety of means. In other embodiments, the detectable response can be generated from as low as 10⁴, 10⁵, or 10⁶ molecular recognition events.

In an embodiment, the invention provides a method for amplifying a molecular recognition interaction between a target and a probe comprising the steps of: contacting the target with the probe under conditions effective to form a target-probe complex; removing target not complexed with the probe (if the probe is attached to the solid surface) or removing probe not complexed with the target (if the target is attached to a solid surface or cell); contacting the target-probe complex with a photoinitiator label under conditions effective to attach the photoinitiator label to the target-probe complex; removing photoinitiator label not attached to the target-probe complex; contacting the photoinitiator-labeled target-probe complex with a polymer precursor solution; exposing the photoinitiator-labeled target-probe complex and the polymer precursor solution to light, thereby forming a polymer; and detecting the polymer formed, thereby detecting an amplified target-probe interaction. The polymer precursor may incorporate a plurality of labeling particles which are incorporated into the polymer formed; the polymer may be detected through detecting a signal from the labeling particles. Excess labeling particles (not incorporated into the polymer) may be removed prior to detection.

In an embodiment, the invention provides a method for identifying a target comprising the steps of: providing a probe array comprising a plurality of different probes, wherein the probes are attached to a solid substrate at known locations (e.g. at a plurality of array spots, each array spot comprising a plurality of probe molecules); contacting the probe array with the target under conditions effective to form a target-probe complex; removing target not complexed with the probe; contacting the target-probe complex with a photoinitiator label under conditions effective to attach the label to the target-probe complex; removing photoinitiator label not attached to the target-probe complex; contacting the photoinitiator-labeled target-probe complex with a polymer precursor solution; exposing the photoinitiator-labeled target-probe complex and the polymer precursor to light, thereby forming a polymer; and detecting the polymer formed, wherein the polymer location indicates the probe which forms a target-probe complex with the target, thereby identifying the target. The polymer precursor may incorporate a plurality of labeling particles which are incorporated into the polymer formed; the polymer may be detected through detecting a signal from the labeling particles. Excess labeling particles (not incorporated into the polymer) may be removed prior to detection.

In an embodiment, the invention provides a method for identifying a target comprising the steps of: providing a probe array comprising a plurality of different probes, wherein the probes are attached to a solid substrate at known locations; contacting the target with the probe array under conditions effective to form a target-probe complex; removing target not complexed with the probe; contacting the target-probe complex with a photoinitiator label under conditions effective to attach the photoinitiator label to the target-probe complex, the photoinitiator label comprising a photoinitiator; removing photoinitiator label not attached to the target-probe complex; contacting the photoinitiator-labeled target-probe complex with a polymer precursor solution comprising a polymer precursor, an optional co-initiator, and a plurality of detectable nanoparticles having a size from 5 to 100 nm; exposing the photoinitiator-labeled target-probe complex and the polymer precursor solution to visible light, thereby forming a polymer gel incorporating detectable nanoparticles; removing and detecting the formation of the polymer gel incorporating detectable nanoparticle by detecting the detectable nanoparticles, removing unpolymerized polymer precursor and detectable nanoparticles not incorporated into the polymer gel, wherein the location of the polymer gel incorporating detectable nanoparticles indicates the probe which forms a target-probe complex with the target, thereby identifying the target.

In an embodiment, the photoinitiator comprises at least one photoinitiator. In an embodiment, the photoinitiator label comprises at least one photoinitiator molecule and a biotin-binding protein, the photoinitiator molecule being attached to the biotin-binding protein. In an embodiment, a photoinitiator molecule can be attached to avidin or streptavidin by modification of avidin or streptavidin lysine residues. For photoinitiators having a carboxylic acid functional group, the carboxylic functional group of the photoinitiator can be coupled to the amine of the lysine residue in the presence of a coupling agent. The result is the formation of a peptide bond between the initiator and the protein. Suitable coupling agents are known to those skilled in the art and include, but are not limited to, EDC. A streptavidin-modified eosin isothiocyanate component (SA-EITC) can be synthesized as follows by functionalizing photoinitiator Eosin-5-Isothiocyanate (Invitrogen) directly onto external lysine residues of streptavidin through formation of a thiourea bond (Hermanson, G. T. Bioconjugate Techniques; Academic Press: San Diego, 1996) (further description provided in US Patent Application Publication US 2009-0137405, hereby incorporated by reference). In an embodiment, the average number of photoinitiator molecules attached to the biotin-binding molecule is between two and three.

In another embodiment, a polymeric photoinitiator label is formed. In the context of cell staining, the polymeric photoinitiator label may be used to detect targets which are not internal to the cell membrane. Such a polymeric photoinitiator label can be formed from a polymer which can be coupled with both a photoinitiator and a molecular recognition group such as a biotin-binding molecule. In another embodiment, the polymeric photoinitiator label can be formed from a polymer which can be coupled with both a photoinitiator and biotin. In an embodiment, the photoinitiator can be attached to the polymer by an ester linkage or by any other kind of linkage known to the art. In an embodiment, the biotin binding protein can be attached to the polymer by an amide linkage. In an embodiment, the polymer comprises an amine functional group or a carboxylic functional group. In an embodiment, the polymer comprises carboxylic acid groups and amide groups. In an embodiment, the polymer comprises a poly(acrylic acid-co-acrylamide) backbone.

In an embodiment, the polymer backbone of the polymeric photoinitiator label comprises sufficient hydrophilic monomeric units that the polymeric label is water soluble. In an embodiment, the hydrophilic monomeric units are selected from the group consisting of ethylene glycol, acrylate, acrylate derivatives such as acrylamide and hydroxyethylacrylate, and vinyl monomers such as 1-vinyl-2-pyrrolidinone. Without wishing to be bound by any particular belief, hydrophilic macroinitiator backbones are believed to limit nonspecific adsorption of the macroinitiator from aqueous solutions.

In an embodiment, a polymeric photoinitiator label can be formed from a polymer which comprises or is coupled to one part of a two-part photoinitiator system. The polymer can be coupled to a molecular recognition group such as avidin or streptavidin. When the combination of the polymer and the second part of the initiator system is exposed to the appropriate wavelength of light, the initiator system is capable of capable of initiating polymerization of a polymer precursor. In different embodiments, the initiator or the co-initiator may be incorporated into the polymer. In an embodiment, one part of the two-part photoinitiator system is a tertiary amine which is part of the polymeric photoinitiator label. The other part of the photoinitiator system can be camphorquinone. (CQ) This two-part system can be activated by light of approximately 469 nm. The tertiary amine can be incorporated into the polymer label by co-polymerizing acrylic acid with a monomer comprising the tertiary amine and an acrylate group.

In an embodiment, the photoinitiator label comprises sufficient photoinitiators so that it may be regarded as a macroinitiator (having many initiators present on a single molecule). The number of initiator groups per label may vary from one label to another. In an embodiment, the use of a macroinitiator can increase the average initiator concentration by a factor of between about 10 to about 100. In other embodiments, the average number of initiators per polymer chain is from 50 to 100, from 80 to 180, from 120 to 160 or from 100 to 200. The number of molecular recognition groups may also vary from chain to chain. In an embodiment, the average number of molecular recognition groups is between one and three. Without wishing to be bound by any particular belief, it is believed that the incorporation of too many initiator groups can lead to nonspecific interaction between the macroinitiator and the array. The molecular weight of the backbone polymer is selected to be large enough to allow attachment of the appropriate number of initiator and molecular recognition groups. For a poly(acrylic acid-co-acrylamide) backbone, the molecular weight of the backbone may be greater than about 50,000.

U.S. Pat. No. 7,354,706 to Rowlen et al. and Sikes et al., 2008, Nat Mat 7:52-56, hereby incorporated by reference, describe experimental procedures for forming polymeric macroinitiators.

As used herein, a photoinitiator is consumed during the polymerization process. In an embodiment, the photoinitiator is a radical photoinitiator. In an embodiment, the photoinitiator may be a unimolecular radical photoinitiator which undergoes homolytic bond cleavage to generate free radicals upon exposure to light. In another embodiment, the photoinitiator is part of a two-part photoinitiator system, comprising a photoinitiator and a co-initiator. In an embodiment, the photoinitiator interacts with the co-initiator to generate free-radicals upon exposure to a source of visible light. The free-radicals can then be used to initiate polymerization of monomers via a chain-growth mechanism. The concept of amplification is inherent in chain-growth polymerization reactions due to the large number of propagation steps that result from a single initiation event. In an embodiment, the amplification factor can be up to 10⁶ or 10⁷.

In an embodiment, the photoinitiator is activated by visible light. Use of visible light sources for photoinitiation has the attractive characteristic of requiring only a low power, inexpensive and mild excitation source. Further, use of visible light has the added advantage of eliminating the unwanted bulk polymerization often observed when using UV light. The use of visible light, rather than UV light, for photoinitiation can also expand the range of suitable monomer formulations. In an embodiment, the monomer formulation contains high concentrations of bi-functional monomers that form thick, highly crosslinked polymer that remains stable at the recognition site or on the surface with rinsing. Finally, visible light can enable more efficient amplification due to its higher penetration capability. In an embodiment, the visible light initiator is a bimolecular photoinitiator (i.e. part of a two-part photoinitiator system).

In an embodiment, the photoinitiator is a photoreducible dye. In an embodiment, the photoinitiator is a xanthene dye. In one embodiment, the photoinitiator molecule is fluorescein or a fluorescein derivative. In an embodiment, the photoinitiator molecule is an eosin, a bromine derivative of fluorescein, or a derivative. In an embodiment, the photoinitiator molecule is 2′,4′,5′,7′-tetrabromofluorescein or a derivative. In an embodiment, the photoinitiator molecule is Rose Bengal or a derivative. In different embodiments, the photoinitiator is activated by wavelengths of light between 400 and 700 nm, between 450 and 600 nm, between 400 and 500 nm, or between 0 and 600 nm. Suitable co-initiators for visible light photoinitiators are known to those skilled in the art. Suitable co-initiators for fluorescein derivatives include, but are not limited to, amines such as methyl diethanol amine and tetraethanol amine. In an embodiment, the visible light activated photoinitiator is water soluble and can be coupled to biotin or a biotin-binding protein.

In an embodiment, the photoinitiator is selected so that the polymer produced through photopolymerization is bound to the surface with sufficient strength that is not easily removed with rinsing. In an embodiment, the photoinitiator molecule is selected so that the polymer formed is attached to the target-probe complex (e.g. target protein-antibody complex) through termination between surface stabilized radicals from the photoinitiator and bulk radicals present on the polymer chains

It is generally accepted that in the eosin-triethanolamine system polymerization occurs as a result of free radicals originating from triethanolamine (Kiziliel, 2004, Langmuir 20, 8652-8658). Though most of the initiating radicals are not surface-tethered, anchoring of the polymer films to the biorecognition site can occur through a variety of methods including the rapid, localized formation of a crosslinked polymer that is physically entangled with the surface from which it is initiated, radical termination reactions that occur by combination of propagating radicals with eosin radicals (Kizilel, 2004, Langmuir 20:8652-8658), and chain transfer of propagating radicals to nearby functional groups along with subsequent reinitiation.

In another embodiment, the photoinitiator is activated by ultraviolet (UV) light. In this embodiment, the monomer formulation is selected so to limit bulk polymerization by the wavelength of UV light. A number of UV-activated photoinitiators are known to the art. UV photoinitiators may be unimolecular photoinitiators or bimolecular photoinitiators. In an embodiment, the UV photoinitiator is a unimolecular photoinitiator which undergoes a-cleavage. In another embodiment, the UV photoinitiator is a bimolecular photoinitiator which undergoes hydrogen abstraction. In another embodiment, the photoinitiator is a cationic photoinitiator. In another embodiment, the photoinitiator comprises a carboxylic acid functional group. Commercially available photoinitiators, for example Irgacure 2959 (Ciba), can be modified to improve their water solubility.

The photoinitiator-labeled complex is typically contacted with a polymer precursor or monomer solution. As used herein, a “polymer precursor” means a molecule or a portion thereof which can be polymerized to form a polymer or copolymer. Such precursors include monomers and oligomers. In this usage a monomer may itself contain a plurality of monomeric units. In an embodiment, the polymer precursor solution comprises at least one polymer precursor, co-initiator and a solvent. In an embodiment, the solvent comprises water and the polymer precursor is water soluble. The polymer precursor solution may also comprise other components, including molecules which serve to accelerate the polymerization reaction (an accelerator). In an embodiment, the concentration of the monomer components is selected to avoid excessive polymer film thickness, thereby improving the resolution of the stain. In an embodiment, the monomer solution is formulated to so that it does not unduly enhance propagation rates and or minimize termination rates, in contrast to formulations more suitable for encapsulation applications.

In an embodiment, the backbone of the monomer comprises sufficient hydrophilic monomeric units that the polymer precursor is water soluble. In an embodiment, the hydrophilic monomeric units are selected from the group consisting of monomers containing ethylene glycol groups (e.g. monomers containing PEG groups), acrylate, acrylate derivatives such as acrylamide and hydroxyethylacrylate, and vinyl monomers such as 1-vinyl-2-pyrrolidinone. In an embodiment, a hydrophilic monomer may contain PEG groups and one or more acrylate or acrylate derivative functional groups. In different embodiments, the molecular weight of the monomer is between 200 and 5000, between 300 and 1000 or from 250-750. In an embodiment, the molecular weight of PEG and/or PEGDA is from 250 to 750. The molecular weight may be measured as the number average molecular weight.

In an embodiment, the polymer precursor solution comprises a multifunctional polymer precursor including at least two polymerizable functional groups. In an embodiment, the multifunctional polymer precursor is a difunctional polymer precursor. In an embodiment the amount of difunctional polymer in the solution is from 5 up to 50 wt % (wt % as compared to the solution as a whole). In different embodiments, the amount of difunctional polymer precursor as compared to the total weight of polymer precursors in solution is at least 25 wt %, 50 wt %, 75 wt %, or 90% wt %. In another embodiment, the relative amount of difunctional polymer precursor may be from 50-100%. The inclusion of substantial amounts of difunctional monomer is believed to aid in the formation of greater amounts of polymer for a given polymerization time. For example, the presence of difunctional acrylate can yield pendant double bonds in propagating polymer chains that may crosslink with other propagating chains, thus suppressing chain termination rates and causing large amounts of high molecular weight polymer to be generated at the molecular recognition site.

In an embodiment, the solution comprises a polymer precursor comprising a plurality of acrylate or acrylate derivative groups. As used herein the polymer precursor comprises acrylate or acrylate derivative groups (such as methacrylate groups). In an embodiment, the solution comprises a difunctional polymer precursor with acrylate or acrylate derivative groups at each end. In an embodiment, the difunctional polymer precursor has a poly(ethylene glycol) (PEG) backbone and acrylate end groups. In an embodiment, the molecular weight of this difunctional PEG monomer is between 300 and 1000. In an embodiment, the weight percent of the difunctional PEG monomer in aqueous solution is from 5% to 50%. In an embodiment, an amine co-initiator is present in a concentration from 22.5 mM to 2250 mM. In an embodiment, the amine co-initiator is water soluble. In an embodiment, the amine co-initiator is methyl diethanol amine. In an embodiment, an accelerant is present in a concentration from greater than zero to 250 nM. In an embodiment, the concentration of vinyl pyrrolidinone and MDEA are 30-40 mM and 200-250 mM, respectively. In an embodiment, the accelerator is 1-vinyl-2-pyrrolidinone.

In another embodiment, the solution comprises a mixture of a difunctional monomer with a vinyl group at each end and a monomer with a single vinyl group. In an embodiment, the polymer precursor solution comprises acrylamide and a bis-acrylamide crosslinker such as N,N′-methylene-bis-acrylamide. As is known to the art, polymerization of these components forms polyacrylamide gel; the structure of the gel (average pore size) is dependent upon the total amount of acrylamide present and the relative amount of cross-linker. In an embodiment, the amount of crosslinker is from 0.5-2 wt %. In an embodiment, the total amount of acrylamide and bisacrylamide is 40 wt % in aqueous solution and 5 mole % (about 2 wt %) of the acrylamide is N,N-methylene-bis-acrylamide. In an embodiment, the amine co-initiator is methyl diethanol amine. In an embodiment, the accelerator is 1-vinyl-2-pyrrolidinone. These acrylamide solutions can be used produce thicker polymer coatings than some of the PEG solutions. In an embodiment, the concentration of vinyl pyrrolidinone and MDEA are 30-40 mM and 200-250 mM, respectively. This formulation is compatible with nitrocellulose-coated glass slides which are desirable for antibody array testing.

In an embodiment, the pH of the polymer precursor solution is greater than 7 and less than or equal to 9. In an embodiment, the pH of the polymer precursor solution is between 8 and 9. Since the pH of the solution can affect free radical formation, it is desirable to control the pH of the solution during the photopolymerization step.

In another embodiment, the polymer precursor solution further comprises microparticles or nanoparticles that can be used to trigger a measurable response and these particles are incorporated into the polymer mass during polymerization. For example, the labeling moieties may be fluorescent, magnetic, radioactive, electrically conducting, absorptive/colored, MRI contrast agents, or electron microscopy contrast agents (e.g. electron opaque particles such as metal particles). As used herein, nanoparticles have an average size greater than or equal to 1 nm and less than 1000 nm. As used herein microparticles have an average size greater than or equal to 1 micron to less than 1000 microns. In an embodiment, the particles are nanoparticles. In different embodiment, the average size of the nanoparticles is from 1 to 500 nm, from 5 to 200 nm, from 5 to 100 nm, from 10 to 50 nm, from 20 to 40 nm or from 10-30 nm. In an embodiment, the average size of the nanoparticles may be larger than the average (calculated) final mesh size of the polymer. In different embodiment, the average size of the nanoparticles may be larger than the average mesh size of the polymer by a factor of up to 2, 5, 7.5 or 10. Too high concentration of fluorescent NPs can cause light attenuation. Too low can result in too little fluorescence. In an embodiment, the concentration is between 0.005 wt % and 5 wt %, from 0.01 to 3 wt %, or from 0.01 to 1 wt %.

In an embodiment, the particles are fluorescent particles. Fluorescent particles known to the art include fluorescently labeled microspheres and nanospheres. These particles include surface labeled spheres, spheres labeled throughout, and spheres possessing at least one internal fluorescent spherical zone (as described in U.S. Pat. No. 5,786,219 to Zhang et al.) Other fluorescent particles known to the art include quantum dots (QDots). These include naturally fluorescent cadmium selenium nanoparticles that have optical properties that are tunable with their size.

In an embodiment of the present invention, the fluorescent particles are microspheres or nanospheres having fluorescent dye substantially contained within the particle, rather than being present only on the surface of the particle. Such particles may also be referred to as having the dye encapsulated within the particles. Such particles are commercially available and are commonly termed microspheres (even for particle diameters less than one micrometer). Containment of the dye within the beads is believed to limit interaction of the dye with the other components of the polymer precursor solution, since physical contact of the dye with these components is limited. In an embodiment, the microspheres or nanospheres are polystyrene particles or beads.

The surface of the microspheres or nanospheres may be modified in a variety of ways. In an embodiment, the microsphere or nanospheres comprise a polystyrene core surrounded by a PEG shell which is then functionalized with the chemical group of interest. Commercially available modifications include carboxylate-modified products, amine-modified products, sulfate and aldehyde-sulfate modified products. In an embodiment, nanospheres used in the present invention are carboxylate modified; the resulting microspheres has been stated to be highly charged and relatively hydrophilic (Molecular Probes Product Information, “Working with FluoSpheres® Fluorescent Microspheres, 1994).

In an embodiment, the surface of the fluorescent particles is selected to allow dispersion of the particles in the polymer precursor solution and subsequent incorporation into the polymer formed at the molecular recognition site. The surface of the fluorescent particles may also be selected to minimize nonspecific recognition between the particles and the cell or the array surface. In an embodiment, the surface of the fluorescent particles is hydrophilic. For polymeric microspheres or nanospheres having a naturally hydrophobic surface, the surface of the polymeric micro or nanospheres may be treated to improve the hydrophilicity of the surface (e.g. carboxylate or amine modified).

In an embodiment, the polymerization product is a polymer gel and the particles are incorporated into the gel. In an embodiment, the network structure of the gel allows encapsulation of the particles without covalent attachment of the particles to the gel network. In another embodiment, the particles are covalently attached to the polymer formed.

Surface treatment of the particles may be used to obtain covalent attachment of the particles to the polymer formed. The appropriate form of surface treatment may vary with the type of particle and the type of polymer, but may include attachment of functional groups, monomers or polymers to the surface. In an embodiment, pendant acrylic monomers may be coupled to the surface of the particles. Acrylic monomers may be coupled to the particle surface by reaction of commercially available particles with acrylate molecules. Polymers may also be attached to the surface through polymerization from the surface.

In an embodiment, the dye contained within particles is selected for compatibility with the photopolymerization process. The absorption spectrum of the dye may overlap that of the initiator, so long as polymerization is not reduced to unacceptable levels. If the absorption spectra overlap, the intensity and/or exposure of the light may be adjusted accordingly to compensate.

In another embodiment, the dye contained within the particles is selected for compatibility with a particular detection device. For example, the dye may be selected so that it has an emission maximum suitable for a particular filter set.

In an embodiment, fluorescent particles suitable for use with fluorescein or fluorescein-derivative initiators can encapsulate dyes having excitation and emission maxima which fall within a relatively broad range of values. In an embodiment, the fluorescent particles can have an absorption/excitation maximum which falls in the range from approximately 500 to approximately 670 nm and an emission maximum which falls in the range from approximately 510 to approximately 690 nm. Suitable fluorescent particles include, but are not limited to, Crimson (excitation/emission maxima of 625/645 nm), Nile Red (broad excitation/emission bandwidths of 535/575 nm), Yellow-Green (excitation/emission maxima 505/515 nm), and Dark Red (excitation/emission maxima of 660/680) FluoSpheres®, all available from Invitrogen.

In an embodiment, the amount of oxygen dissolved in the polymer precursor solution is minimized to minimize oxygen inhibition of the polymerization process. The amount of oxygen dissolved in the solution may be minimized by control of the atmosphere under which polymerization takes place, reducing the oxygen content of the polymer precursor solution by flowing a gas through it, or by a combination thereof. Suitable atmospheres and purge gases include, but are not limited to, argon and nitrogen. The amount of oxygen dissolved in the polymer precursor solution may also be controlled by the addition of oxygen inhibition agents.

In some embodiments of the invention, the photoinitiator-labeled target-probe complex (e.g. the photoinitiator-labeled target protein-antibody complex) and polymer precursor solution are exposed to light, thereby forming a polymer. Photopolymerization occurs when polymer precursor solutions are exposed to light of sufficient power and of a wavelength capable of initiating polymerization. In an embodiment, the light source primarily provides light having a wavelength between 400 and 700 nm. In an embodiment, the intensity of the radiation is selected so that an appropriate dose of radiation can be delivered in less than or equal to one-half hour. In different embodiments, the intensity of the radiation is from 1-50 or 5-50 mW/cm².

In an embodiment, the polymer formed is a covalently crosslinked hydrogel. The term “hydrogel” refers to a class of polymeric materials which are extensively swollen in an aqueous medium, but which do not dissolve in water. Determination of polymer formation may be made with either swollen or dried gels. For accurate polymer film thickness measurements, the gels are typically dried.

In different embodiments, the methods of the invention are capable of detecting as few as 10³ or ˜10⁴ labeled target molecules using minimal instrumentation, such as an optical microscope or CCD camera. In other embodiments, the methods of the invention are capable of visual detection of concentrations as few as 100, 50, 25, 10, 5, 1, 0.5, 0.1 or 0.005 biomolecules/μm².

In an embodiment, the improvement in sensitivity of the methods of the invention can be quantified as the ratio of the amount of fluorescence resulting from incorporation of fluorescent nanoparticles into the polymer mass to the amount of fluorescence resulting from a fluorescent labeling moiety being directly coupled to each probe. For this measurement, the fluorescent labeling moiety is typically selected so that it has similar spectral properties to the fluorescent nanoparticles. For example, fluorescence signal of staining with polymerization-based amplification with yellow/green NPs can be compared to that of staining done with streptavidin-FITC, since FITC and the yellow/green NPs have similar spectral properties (e.g. excitation wavelengths), allowing them to be imaged by the same instrumentation.

In an embodiment, the “overall gain” for a given color of nanoparticle and fluorescence measurement technique may be measured as the slope on the plot of photoinitiator surface density (x) vs. polymer film fluorescence (y) and is indicative of the increase in film fluorescence observed as photoinitiator surface density increases. Similarly, for a given color of nanoparticle and fluorescence measurement technique the “fluorescence gain” denotes the slope on the plot of film thickness (x) vs. film fluorescence (y) and signifies the amount of increase in film fluorescence observed with an increase in film thickness. The values of overall gain and/or fluorescence gain (for a given color of nanoparticle and fluorescence measurement technique) can be optimized to optimize polymerization conditions.

In one aspect, the methods of the invention rely on biorecognition between a target protein and an antibody. In order for molecular interaction between the target protein and the antibody to identify the target protein, the molecular interaction is sufficiently specific.

The antibody may be selected for complex formation with a particular antigen or epitope of an antigen. The antibody may be a monoclonal or polyclonal antibody. The different embodiments, the antibody may be selected from the class IgA, IgD, IgE, IgG or IgM. The antibody may be conjugated with a biotin binding protein or a photoinitiator. In an embodiment, the biotin-binding protein comprises avidin, streptavidin, or Neutravidin (a deglycosylated form of avidin).

Techniques for formation of antibody-antigen complexes are known to those skilled in the art. Typically, the antibody is supplied in a buffer containing a nonspecific protein such as bovine serum albumin (BSA). The antibody solution may be an aqueous solution and may be contacted with the sample containing the antigen for a period of time typically referred to as the incubation time. In an embodiment, the concentration of antibody in the solution is selected to be below levels which promote nonspecific staining. Commercial antibody solutions may vary in concentration, and are typically diluted before use. For example a stock solution having a concentration in the mg per mL range may be diluted to a range of micrograms per mL. In different embodiments, the initial concentration of the stock solution may be from 0.5 to 10 mg/mL or 0.5 to 10 μg/mL. In different embodiments, the dilution of the primary antibody solution using the methods of the present invention may be from 1:100 to 1:500,000, 1:1000 to 1:500,000, 1:5,000 to 1:500,000, 1:10,000 to 1:500,000, 1:10,000 to 1:100,000, 1:1000 to 1:100,000, or 1:1000 to 1:10,000. In contrast dilution ranges for conventional technology may be diluted between 1:0 (no dilution) up to ˜1:1000. The sample is typically washed following this step.

If a secondary antibody is used, it is typically selected for formation of a complex with the primary antibody. The secondary antibody may be raised in one species against antibodies of another species (the species of the primary antibody). For example, if the primary antibody is produced in mouse, an anti-mouse antibody produced in goat may be used as the secondary antibody. The secondary antibody may be conjugated with a biotin binding protein or a photoinitiator. Typically, the secondary antibody is also supplied in a solution containing a nonspecific protein, which is contacted with the antibody of the antibody-antigen complex. This solution may be an aqueous solution. The concentration of secondary antibody in the solution is usually selected to be below levels which promote nonspecific staining. The sample is typically washed following this step.

In an embodiment, the primary antibody is coupled to a photoinitiator. In an embodiment, the primary antibody may be biotin-labeled and the photoinitiator coupled to a biotin-binding protein. The photoinitiator may also be covalently bound to the primary antibody. In another embodiment, the primary antibody is coupled to a secondary antibody, which in turn is coupled to the photoinitiator. The secondary antibody may be biotin-labeled and photoinitiator coupled to a biotin-binding protein or the secondary antibody may be covalently bound to the photoinitiator.

In an embodiment of the method for detecting the molecular recognition event, a photoinitiator-labeled target-antibody complex is formed. The photoinitiator-labeled target-antibody complex may be a photoinitiator-labeled target protein-antibody complex. The photoinitiator-labeled target protein antibody complex comprises a target protein, a primary antibody, and a photoinitiator. The complex may also optionally include a secondary antibody which undergoes molecular recognition with the primary antibody. In an embodiment, the molecular recognition event occurs between the target protein and the primary antibody followed by coupling of the photoinitiator to the primary antibody. In another embodiment, the molecular recognition may occur after coupling of the photoinitiator to the primary antibody.

Upon exposure to light, the photoinitiator initiates polymerization of a monomer or polymer precursor solution in contact with the photoinitiator label of the photoinitiator-labeled target probe complex (e.g. target protein-antibody complex). In an embodiment, a cross-linked polymer is formed. In an embodiment, the polymer mass is bound to the target probe complex (e.g. target protein-antibody complex), forming a polymer label. When labeling moieties are incorporated into the polymer label, detection of these particles in the polymer label allows detection of the molecular recognition event.

In an embodiment, after polymerization, unpolymerized polymer precursor and labeling moieties/particles not incorporated into the polymer label are removed prior to the detection step. The unpolymerized polymer precursor and unincorporated labeling moieties may be removed by rinsing, for example by rinsing with water or an aqueous solution.

In an embodiment, the photoinitiator-labeled target protein-antibody complex is formed by covalently attaching the photoinitiator to either the primary or secondary antibody. In another embodiment, the photoinitiator-labeled target protein-antibody complex is formed by attaching a photoinitiator label comprising a photoinitiator and a biotin binding protein to a biotin-labeled primary or secondary antibody present in the target-protein antibody complex. The photoinitiator label will comprise a photoinitiator, but in some embodiments the photoinitiator label can comprise the co-initiator portion of a two part initiation system.

In an embodiment, the target protein-antibody complex is contacted with the photoinitiator label by contacting the target protein-antibody complex with a photoinitiator label solution comprising the photoinitiator labels. In an embodiment, the solvent is aqueous and the photoinitiator label is water soluble. In an embodiment, the concentration of the photoinitiator label in the solution may be selected to limit nonspecific adsorption of the photoinitiator label to the surface of a substrate. After the photoinitiator label is attached to the target protein-antibody complex, excess photoinitiator label may be removed. In an embodiment, photoinitiator label not attached to the target protein-antibody complex is removed to sufficiently reduce the any signal resulting from non-specific adsorption. The excess photoinitiator label may be removed by rinsing. The rinse may be an aqueous solution.

Application of this method to adherent cells and tissue sections can be used to visualize the spatial localization of target proteins by light microscopy, fluorescent microscopy, confocal microscopy and electron microscopy. An application of this method can be utilized for the labeling of cellular proteins, both on the cell surface and within the cell, for detection by flow cytometry for fluorescent activated cell sorting (FACS) and analysis. This technique may also be applied for in vivo and intravital (within the body) identification and visualization of target proteins in whole living organisms.

The methods of the invention can be used for staining a variety of cell structures, including small localized protein structures on the nuclear envelope and large regions of cytoplasm. In different embodiments, the cells to be stained may be adherent cells or nonadherent cells, live or fixed. The methods and kits of the invention may also be used with cells present in tissue sections or in tissue engineered scaffolds. The cells or tissues may be from any species; the methods of the invention need not be specific for human tissue. Also, the methods and kits of the invention can be applied to micro-organisms such as bacteria or fungi.

The cells may be subjected to fixation treatment prior to staining. Suitable fixation procedures are known to those skilled in the art. Typically, the fixation treatment will be selected to preserve the primary antigens of interest. Fixation agents known to the art include organic solvents (e.g. methanol) and crosslinking reagents. If access to antigens in the interior of the cell is desired, the cells also may be subjected to a permeabilization treatment. Such permeabilization treatments are known to those skilled in the art and can involve use of a detergent. The cells may also be treated with a blocking reagent prior to contact with the antibody solution. The high sensitivity of the methods of the invention may allow for more tolerance in fixing or permeabilization conditions. Better sensitivity means that detection can be achieved with fewer intact and accessible epitopes.

In an embodiment, the invention provides a kit suitable for use with a biotin-labeled target protein-antibody complex, the kit comprising a first solution including a conjugate of a photoinitiator and a biotin binding protein, a second solution including a monomer, and a plurality of labeling moieties. The first solution may be an aqueous solution comprising a conjugate of the biotin binding protein and the photoinitiator. The photoinitiator conjugate may be a streptavidin-photoinitiator conjugate. The second solution may be an aqueous solution comprising the monomer, a co-initiator, and a polymerization accelerator. The monomer may be difunctional polyethylene glycol monomer such as polyethylene glycol diacrylate. In an embodiment, the co-initiator is a tertiary amine such as methyldiethanol amine. In an embodiment, the accelerator is 1-vinyl-2-pyrrolidinone. The labeling moieties may be nanoparticles. In an embodiment, a nanoparticle solution is mixed after purchase of the kit. In another embodiment, an aqueous nanoparticles solution may be provided as part of the kit.

In an embodiment, a kit for polymerization-based detection includes:
a) streptavidin-initiator conjugate solution (1 mL at approximately 0.5 mg/mL, balance is water with buffer)
b) monomer mix (50 mL)
   25 volume % polyetheylene glycol diacrylate (MW 575)
   225 mM methyldiethanol amine
   37 mM 1-vinyl-2-pyrrolidinone
   balance is water

c) nanoparticles (˜1 mL, ˜2% solids, balance is water with buffer, but concentration and amount dependent on which nanoparticles are used)

In an embodiment, a kit suitable for use with a target protein-antibody complex comprises a first solution including a conjugate of a photoinitiator and a secondary antibody, a second solution including a monomer, and a plurality of labeling moieties. The first solution may be an aqueous solution comprising a conjugate of the photoinitiator and the secondary antibody. The second solution may be an aqueous solution comprising the monomer, a co-initiator, and a polymerization accelerator. The monomer may be difunctional polyethylene glycol monomer such as polyethylene glycol diacrylate. In an embodiment, the co-initiator is a tertiary amine such as methyldiethanol amine. In an embodiment, the accelerator is 1-vinyl-2-pyrrolidinone. The labeling moieties may be nanoparticles. In an embodiment, a nanoparticle solution is mixed after purchase of the kit. In another embodiment, an aqueous nanoparticles solution may be provided as part of the kit.

In another embodiment, a kit for polymerization-based detection includes:
a) secondary antibody-initiator conjugate (1 mL at approximately 0.5 mg/mL, balance is water with buffer)
b) monomer mix
   25 volume % polyetheylene glycol diacrylate (MW 575)
   225 mM methyldiethanol amine
   37 mM 1-vinyl-2-pyrrolidinone balance is water
c) nanoparticles (˜1 mL, ˜2% solids, balance is water with buffer, but concentration and amount dependent on which nanoparticles are used).

In another embodiment, the invention provides a kit comprising:
a) a first aqueous solution including a conjugate of a photocleavable photoinitiator and biotin-binding protein;
b) a second aqueous solution including a biotinylated secondary antibody;
c) a third aqueous solution including a water soluble monomer and a polymerization accelerator; and
d) a fourth aqueous solution including fluorescent nanoparticles.
   The photocleavable photoinitiator and the biotin-binding protein may be attached to a polymer to form a macroinitiator.

In an embodiment, either the target or the probe is a nucleic acid. In an embodiment, both the target and the probe are a single stranded nucleic acid. In an embodiment, the probe is an oligonucleotide, a relatively short chain of single-stranded DNA or RNA. “Nucleic acid” includes DNA and RNA, whether single or double stranded. The term is also intended to include a strand that is a mixture of nucleic acids and nucleic acid analogs and/or nucleotide analogs, or that is made entirely of nucleic acid analogs and/or nucleotide analogs and that may be conjugated to a linker molecule. “Nucleic acid analogue” refers to modified nucleic acids or species unrelated to nucleic acids that are capable of providing selective binding to nucleic acids or other nucleic acid analogues. As used herein, the term “nucleotide analogues” includes nucleic acids where the internucleotide phosphodiester bond of DNA or RNA is modified to enhance bio-stability of the oligomer and “tune” the selectivity/specificity for target molecules (Uhlmann, et al., (1990), Angew. Chem. Int. Ed. Eng., 90: 543; Goodchild, (1990), J. Bioconjugate Chem., I: 165; Englisch et al., (1991), Angew, Chem. Int. Ed. Eng., 30: 613). Such modifications may include and are not limited to phosphorothioates, phosphorodithioates, phosphotriesters, phosphoramidates or methylphosphonates. The 2′-O-methyl, allyl and 2′-deoxy-2′-fluoro RNA analogs, when incorporated into an oligomer show increased biostability and stabilization of the RNA/DNA duplex (Lesnik et al., (1993), Biochemistry, 32: 7832). As used herein, the term “nucleic acid analogues” also include alpha anomers (α-DNA), L-DNA (mirror image DNA), 2′-5′ linked RNA, branched DNA/RNA or chimeras of natural DNA or RNA and the above-modified nucleic acids. For the purposes of the present invention, any nucleic acid containing a “nucleotide analogue” shall be considered as a nucleic acid analogue. Backbone replaced nucleic acid analogues can also be adapted to for use as immobilized selective moieties of the present invention. For purposes of the present invention, the peptide nucleic acids (PNAs) (Nielsen et al., (1993), Anti-Cancer Drug Design, 8: 53; Engels et al., (1992), Angew, Chem. Int. Ed. Eng., 31: 1008) and carbamate-bridged morpholino-type oligonucleotide analogs (Burger, D. R., (1993), J. Clinical Immunoassay, 16: 224; Uhlmann, et al., (1993), Methods in Molecular Biology, 20, “Protocols for Oligonucleotides and Analogs,” ed. Sudhir Agarwal, Humana Press, NJ, U.S.A., pp. 335-389) are also embraced by the term “nucleic acid analogues”. Both exhibit sequence-specific binding to DNA with the resulting duplexes being more thermally stable than the natural DNA/DNA duplex. Other backbone-replaced nucleic acids are well known to those skilled in the art and can also be used in the present invention (See e.g., Uhlmann et al., (1993), Methods in Molecular Biology, 20, “Protocols for Oligonucleotides and Analogs,” ed. Sudhir Agrawal, Humana Press, NJ, U.S.A., pp. 335).

More generally, the probe and/or target can be an oligomer. “Oligomer” refers to a polymer that consists of two or more monomers that are not necessarily identical. Oligomers include, without limitation, nucleic acids (which include nucleic acid analogs as defined above), oligoelectrolytes, hydrocarbon based compounds, dendrimers, nucleic acid analogues, polypeptides, oligopeptides, polyethers, oligoethers any or all of which may be immobilized to a substrate. Oligomers can be immobilized to a substrate surface directly or via a linker molecule.

In an embodiment, the probe is DNA. The DNA may be genomic DNA or cloned DNA. The DNA may be complementary DNA (cDNA), in which case the target may be messenger RNA (mRNA). The DNA may also be an Expressed Sequence Tag (EST) or a Bacterial Artificial Chromosome (BAC). For use in hybridization microarrays, double-stranded probes are denatured prior to hybridization, effectively resulting in single-stranded probes.

In an embodiment, the target is genetic material from influenza A, B, or C. Influenza is an orthomyxovirus with three genera, types A, B, and C. The types are distinguished by the nucleoprotein antigenicity (Dimmock, N. J., Easton, A. J., Leppard, K. N. (2001) “Introduction to Modern Virology” 5^{th} edition, Blackwell Science Ltd., London). Influenza A and B each contain 8 segments of negative sense ssRNA. Type A viruses can also be divided into antigenic sub-types on the basis of two viral surface glycoproteins, hemagglutinin (HA) and neuraminidase (NA). There are currently 15 identified HA sub-types (designated H1 through H15) and 9 NA sub-types (N1 through N9) all of which can be found in wild aquatic birds (Lamb, R. A. & Krug, R. M., (1996) “Orthomyxoviridae: The Viruses and their Replication, in Fields Virology”, B. N. Fields, D. M. Knipe, and P. M. Howley, Editors. Lippincott-Raven: Hagerstown). Of the 135 possible combinations of HA and NA, only four (H1N1, H1N2, H2N2, and H3N2) have widely circulated in the human population since the virus was first isolated in 1933. The two most common sub-types of influenza A currently circulating in the human population are H3N2 and H1N1. LI et al. describe a DNA microarray whose probes were multiple fragments of the hemagglutinin, neuraminidase, and matrix protein genes. (Li, J. et al., (2001), J. Clinical Microbio., 39(2), 696-704).

DNA microarrays are known to the art and commercially available. The general structure of a DNA microarray is a well defined array of spots on an optically flat surface, each of which contains a layer of relatively short strands of DNA. As referred to herein, microarrays have a spot size less than about 1.0 mm. In most hybridization experiments, 15-25 nucleotide sequences are the minimum oligonucleotide probe length (Schena, M., “Microarray Analysis, (2003) John Wiley & Sons, New Jersey, p. 8). The substrate is generally flat glass primed with an organosilane that contains an aldehyde functional group. The aldehyde groups facilitate covalent bond formation to biomolecules with free primary amines via Schiff base interactions. After reaction the chip is cured to form a very stable array ready for hybridization.

Protein microarrays are also known to the art and some are commercially available. The general structure of protein microarrays can be similar to that of DNA microarrays, except that array spots can contain antibodies (in particular monoclonal antibodies), antigens, recombinant proteins, or peptides. For accurate measurement of binding events, surface-bound proteins must be correctly folded and fully functional (Constans, A., 2004, The Scientist, 18(15) 42). To reduce protein unfolding, the proteins can be protected by use of stabilizing buffers and/or relatively high protein concentrations (Schena, M., “Microarray Analysis, (2003) John Wiley & Sons, New Jersey, p. 154). To avoid the protein folding problem, the functional domains of interest can be arrayed rather than the whole protein, forming domain-based arrays (Constans, 2004, ibid).

In an embodiment, the probe is contacted with a solution comprising the target under conditions effective to form a target-probe complex. The conditions effective to form a target-probe complex depend on the target and probe species. For ssDNA or RNA targets binding to ssDNA probes, suitable hybridization conditions have been described in the scientific literature. In an embodiment, it is sufficient to contact a solution comprising the target with the probe for about 2 hours at about 42° C. In an embodiment, this solution also comprises an agent, such as a crowding agent, to limit nonspecific interactions. With reference to nucleic acid interactions, a crowding agent is an agent that interrupts nonspecific adsorption between nucleic acids that are not complementary. Formamide is one such agent to limit nonspecific interactions (Stahl, D. A., and R. Amann. 1991. Development and application of nucleic acid probes, p. 205-248. In E. Stackebrandt and M. Goodfellow (ed.), Nucleic acid techniques in bacterial systematics. John Wiley & Sons Ltd., Chichester, United Kingdom). Nonspecific interactions can also be limited by applying a blocking agent to the microarray prior to contacting the target with the probe. Suitable blocking agents are known to the art and include, but are not limited to bovine serum albumin (BSA), nonfat milk, and sodium borohydride. Detergents such as sodium lauroyl sarcosine or sodium dodecyl sulfate can also be added to aldehyde surface hybridization reactions to reduce background (Schena, M., “Microarray Analysis, (2003) John Wiley & Sons, New Jersey, p. 117). The target solution may also be contacted with the probe at higher temperatures in order to limit nonspecific interactions.

After the target is contacted with the probe, targets which have not formed target-probe complexes are removed. The unbound targets can be removed through rinsing. Water or an aqueous solution may be used for rinsing away unbound targets.

In an embodiment, the substrate surface is treated to minimize nonspecific adsorption of the photoinitiator label. If the initiator is to be attached through biotin-avidin interaction, a blocking agent can be applied to the microarray to limit nonspecific interaction of avidin. Suitable blocking agents are known to the art and include, but are not limited to, bovine serum albumin (BSA), nonfat milk and sodium borohydride. PEG-based blocking agents which react with amine functionalities are also known to the art. The blocking agent may be applied to the substrate surface prior to contact of the photoinitiator label solution with the substrate, may be supplied in the photoinitiator label solution, or both. Denhardt's solution is a commercially available solution (Sigma-Aldrich) which contains BSA and can be included in the photoinitiator label solution. In an embodiment, the array is incubated with the blocking agent for approximately 20 minutes at about room temperature.

In an embodiment, the target-probe complex is contacted with a photoinitiator label under conditions effective to attach the photoinitiator label to the target probe complex. In an embodiment, the target-probe complex is contacted with the photoinitiator label by contacting the target-probe complex with a photoinitiator label solution comprising the photoinitiator label. In an embodiment, the solvent is aqueous and the photoinitiator label is water soluble. In an embodiment, the concentration of the photoinitiator label in the solution may be selected to limit nonspecific adsorption of the photoinitiator label to the surface of a substrate.

In an embodiment, the invention provides a method of detecting a molecular recognition event between an antibody and a target protein in the interior of a cell, the cell being attached to a substrate, the method comprising the steps of:
a) subjecting the cell to a fixation step;
b) treating the cell with a blocking agent;
c) contacting the antibody with the target protein under conditions effective to form an antibody-target protein complex;
d) removing antibody not complexed with the target protein;
e) labeling the antibody-target protein complex with a photoinitiator label wherein the photoinitiator label comprises a photoreducible dye photoinitiator;
f) removing photoinitiator label not attached to the antibody-target protein complex;
g) contacting the photoinitiator-labeled antibody-target protein complex with a polymer precursor solution comprising a water soluble monomer, an amine co-initiator, and plurality of fluorescent nanoparticles, wherein the average size of the fluorescent particles is from 10 to 50 nm;
h) exposing the photoinitiator-labeled antibody-target protein complex and the polymer precursor solution to visible light, thereby forming a polymer gel attached to the cell and incorporating the fluorescent nanoparticles;
i) removing unpolymerized polymer precursor and fluorescent nanoparticles not incorporated into the polymer gel, and
j) detecting fluorescence from the nanoparticles in the polymer gel, thereby detecting the molecular recognition event.
wherein the ratio of the fluorescence signal from nanoparticles in the polymer gel to a background fluorescence signal is greater than 5.

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claim.

Every formulation or combination of components described or exemplified can be used to practice the invention, unless otherwise stated. Specific names of compounds are intended to be exemplary, as it is known that one of ordinary skill in the art can name the same compounds differently. When a compound is described herein such that a particular isomer or enantiomer of the compound is not specified, for example, in a formula or in a chemical name, that description is intended to include each isomers and enantiomer of the compound described individual or in any combination. One of ordinary skill in the art will appreciate that methods, device elements, starting materials, and synthetic methods, other than those specifically exemplified can be employed in the practice of the invention without resort to undue experimentation. All art-known functional equivalents, of any such methods, device elements, starting materials, and synthetic methods, are intended to be included in this invention. Whenever a range is given in the specification, for example, a temperature range, a time range, or a composition range, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention.

In general the terms and phrases used herein have their art-recognized meaning, which can be found by reference to standard texts, journal references and contexts known to those skilled in the art. The preceding definitions are provided to clarify their specific use in the context of the invention.

### Example 1

Immunofluorescent staining is central to nearly all cell-based research, yet only a few fluorescent signal amplification approaches for cell staining exist, each with distinct limitations. Here, we present a novel, fluorescent polymerization-based amplification method (FPBA) that is shown to enable similar signal intensities as the highly sensitive, enzyme-based tyramide signal amplification (TSA) approach. Being non-enzymatic, FPBA is not expected to suffer from non-specific staining of endogenous enzymes, as occurs with enzyme-based approaches. FPBA employs probes labeled with photopolymerization initiators which lead to the controlled formation of fluorescent polymer films only at targeted biorecognition sites. Nuclear pore complex proteins (NPC) (in membranes), vimentin (in filaments), and von Willebrand factor (in granules) were all successfully immunostained by FPBA. Also, FPBA was demonstrated to be capable of multicolor immunostaining of multiple antigens. To assess relative sensitivity, decreasing concentrations of anti-NPC antibody were utilized, indicating that both FPBA and TSA stained NPC down to a 1:100,000 dilution. Non-specific, cytoplasmic signal resulting from NPC staining was found to be reduced up to 5.5-fold in FPBA as compared to TSA, demonstrating better signal localization with FPBA. FPBA's unique approach affords a combination of preferred attributes including high sensitivity and specificity not otherwise available with current techniques.

### Introduction

Immunofluorescent staining of cells is a central technology in cell biology that is invaluable for localization of cellular proteins, elucidating cellular functions and aiding in pathology-based disease diagnosis. To localize fluorescent signals at antigenic biorecognition sites, an organic fluorophore is typically coupled to the primary or secondary antibody. In a similar approach, a biotinylated antibody probe is used, followed by fluorescently labeled avidin which specifically binds biotin. Though straightforward to implement, direct labeling of protein probes is particularly inadequate for detection of low abundance antigens (Van heusden et al. 1997, J Histochem Cytochem 45:315-319). Enzymatic fluorescent signal amplification techniques which utilize an enzyme to deposit fluors near biorecognition sites enable significantly improved detection limits and are commercially available, yet they also have limitations. One popular enzymatic approach is tyramide signal amplification (TSA), also referred to as catalyzed reporter deposition (CARD), in which probes are coupled to horseradish peroxidase (HRP) which catalyzes the formation of short-lived fluorescently-labeled tyramide radicals that rapidly react with chemical groups near the enzyme, thereby immobilizing the fluorophores. Though enabling highly sensitive detection, TSA is often hindered by loss of signal localization as the tyramide radicals diffuse away from the biorecognition site prior to immobilization (Kerstens et al. 1995, J Histochem Cytochem 43:347-352; Wiedorn et al. 1999, Histochem Cell Biol 111:89-95). Additionally, non-specific staining from endogenous cellular peroxidases is often difficult or impractical to eliminate completely (Hunyady et al. 1996, Histochem. Cell Biol. 106, 447-456; Ishii et al. 2004, FEMS Microbiol Ecol 50:203-212; Pavlekovic et al. 2009, J Microbiol Methods 78:119-126). Another enzymatic method, enzyme-linked fluorescence (ELF) generates a precipitating fluorescent substrate but is only available with a single fluorophore (Larison et al. 1995, Histochem. Cytochem. 43, 77-83). As an alternative to enzymatic approaches, probe-functionalized quantum dots (QDs) have also been employed for cellular immunostaining (Wu et al. 2003, Nat Biotechnol 21:41-46). The QD approach yields an exceptionally photostable stain and also provides especially narrow emission spectra, reducing cross-talk in multiplexed staining of multiple antigens. Though QDs are brighter than organic fluorophores, they typically do not generally achieve the same increase in fluorescence intensity as achieved by enzyme-based methods (Speel et al. 1999, J Histochem Cytochem 47:281-288; Wu et al. 2003, Nat Biotechnol 21:41-46).

New signal amplification approaches that achieve the high sensitivity and strong fluorescence intensities required for low abundance protein detection, without suffering from diffusion-related loss of signal localization and non-specific staining of endogenous cellular enzymes that occur with enzymatic amplification methods can improve upon the currently available methodologies for fluorescent cellular immunostaining. Here, we present a novel non-enzymatic, polymerization-based signal amplification method for immunofluorescent staining of cells that combines the attributes of high sensitivity, good signal localization, photostable fluorescence, and multiplexed staining ability, without interference from endogenous enzymes or the need for enzymatic processes.

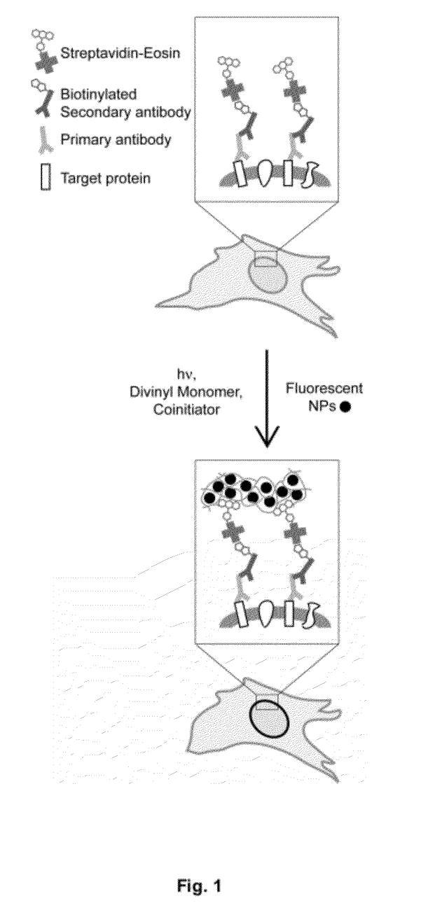

Harnessing the amplification inherent in radical polymerization reactions, photoinitiator molecules are coupled to biological probes such that light exposure initiates the conversion of monomer and fluorescent moieties into a highly fluorescent polymer film localized at the biorecognition site (FIG. 1). Specifically, this fluorescent polymerization-based signal amplification method (FPBA), utilizes protein probes labeled with eosin, a photosensitizing initiator. After unbound eosin-labeled probe is removed, a monomer solution comprised of poly(ethylene glycol) diacrylate (PEGDA) monomer, fluorescent nanoparticles (NPs), vinyl pyrrolidone as a comonomer, and N-methyldiethanolamine (MDEA) as a coinitiator is applied to the sample. The surface is then irradiated with visible light which causes eosin on the biological probe to undergo energy, charge, and electron transfer with MDEA to generate initiating MDEA radicals that propagate through the monomer to yield a crosslinked polymer network (Avens and Bowman 2009, J Polym Sci Part A: Polym Chemistry 47:6083-6094)

As the crosslinked polymer network forms, the fluorescent NPs in the monomer solution become entrapped wherever polymerization has occurred. After polymerization, the surface is rinsed to remove unreacted monomer and NPs that did not become entrapped in the polymer film. In this manner, FPBA yields highly fluorescent polymer films formed only at the biorecognition sites.

A PEGDA-based monomer system was chosen because it is water soluble, it was found to generate films that entrap a higher density of fluorescent NPs than other formulations investigated, and it yields films that are appropriately thin so as to favor localized staining. Though most of the initiating radicals are not surface-tethered, anchoring of the polymer films to the biorecognition site occurs through a variety of methods including the rapid, localized formation of a crosslinked polymer that is physically entangled with the surface from which it is initiated, radical termination reactions that occur by combination of propagating radicals with eosin radicals (Kizilel et al. 2004, Langmuir 20:8652-8658), and chain transfer of propagating radicals to nearby functional groups followed by reinitiation. The films are not displaced during aqueous wash steps. Moreover, the polymer film is covalently crosslinked, rendering it resistant to chemical and physical perturbations and practically eliminating diffusion of the fluorescent NPs out of the films. The rate of film growth depends on the radical initiation rate, which is determined by the light intensity and the surface density of eosin initiators. As there is minimal attenuation of visible light through cells, the PEGDA films are expected to demonstrate uniform three-dimensional growth from cellular structures where the eosin-labeled probe has been selectively bound.

Compared to individual fluorophores, the fluorescent NPs employed here for FPBA exhibit enhanced photostability (Mayr et al. 2009, J Fluoresc 19:303-310) and undergo fewer unwanted side reactions during the polymerization (Avens and Bowman, 2010, Acta Biomater 6:83-89). Specifically, FPBA was performed using FluoSpheres (commercially available from Invitrogen) which are polystyrene NPs with carboxylate surface functionalities that have fluorophores embedded in the interior. Since the fluorophores are embedded within the polystyrene environment, they are relatively shielded from problematic side reactions such as photobleaching and non-specific photoinitiation (Avens and Bowman 2010, Acta Biomater 6:83-89).

In previously published reports, polymerization-based signal amplification (PBA) in a variety of configurations has been demonstrated to be a sensitive and valuable signal amplification approach for surface-based biosensing (Lou et al. 2005, Anal Chem 77:4698-4705; Hansen et al. 2008, Biomacromolecules 9:355-362; Hansen et al. 2008, Anal Bioanal Chem 392:167-175; He et al. 2008, Anal Chem 80:3633-3639; Sikes et al. 2008, Nat Mat 7:52-56; Hansen et al. 2009, Anal Biochem 386:285-287; Sikes et al. 2009, Lab on a Chip 9:653-656; Avens and Bowman 2010, Acta Biomater 6:83-89), though its application to biodetection and immunostaining of cells has never before been reported. Non-fluorescent PBA has been shown to enable instrument-free detection of as few as ˜1000 surface-bound biomarkers (Sikes et al. 2008, Nat Mat 7:52-56). Additionally, in an antibody microarray format, FPBA has been demonstrated to yield a 100-fold improvement in sensitivity compared to the use of fluor-labeled streptavidin (Avens and Bowman 2010, Acta Biomater 6:83-89). These dramatic improvements in sensitivity associated with PBA have been found to be well-suited for extension to immunofluorescent staining where, for reasons of reducing costs, improving sensitivity, decreasing non-specific staining, and simplifying procedures, there is a strong desire for improved methodologies.

FPBA was developed and evaluated in this work for immunofluorescent staining of cellular antigens in cultured cells. In the work presented here, eosin was coupled to streptavidin to create a generalized approach for detecting a variety of biotinylated targets; however, coupling eosin to a primary or secondary antibody probe is readily achievable and would enable fewer immunostaining steps. FPBA was compared to Alexa488-labeled streptavidin (SA-Alexa488) for the detection of vimentin, von Willebrand factor (vWF), and nuclear pore complex proteins (NPC). The staining pattern, specificity and resolution were evaluated and compared for each of these targets. Additionally, the relative sensitivities of FPBA, TSA, and SA-Alexa488 were evaluated in the context of NPC staining by using decreasing concentrations of primary antibody. Use of low primary antibody concentrations typically results in fewer biorecognition events, thereby facilitating a comparison of the relative sensitivities of the three staining methods. Since FPBA and SA-Alexa488 both employed the streptavidin-biotin approach, a TSA kit was selected as a control which utilized horse radish peroxidase coupled to streptavidin (SA-HRP) in conjunction with Alexa488-labeled tyramide. FPBA's capability for multicolor detection of two antigens was evaluated using two sequential FPBA steps, each employing differently colored fluorescent NPs. Finally, the photostability of FPBA was evaluated and compared to the photostability of SA-Alexa488 and FITC-labeled streptavidin (SA-FITC) staining.

### Materials and Methods

Materials. Monoclonal mouse IgG1 anti-nuclear pore complex (NPC) (MAb414) purchased from Covance (Princeton, N.J.) (catalogue #MMS-120P). Polyclonal rabbit anti-von Willebrand Factor (vWF) was purchased from Dako (Denmark) (catalogue #A0082). Biotinylated polyclonal goat IgG anti-mouse IgG (H+L) (catalogue #BA-9200), biotinylated polyclonal goat IgG anti-rabbit IgG (H+L) (catalogue #BA-1000), streptavidin fluorescein (SA-FITC), normal horse serum, and Vectashield hard set mounting medium for fluorescence were purchased from Vector Laboratories (Burlingame, Calif.). 50×Denhardt's solution, streptavidin-Alexa Fluor 488 (SA-Alexa488), and TSA kit with HRP-streptavidin (SA-HRP) and Alexa Fluor488 tyramide were purchased from Invitrogen (Carlsbad, Calif.). Hydromount was purchased from Life Science Products (Frederick, Colo.). Bovine serum albumin (BSA) was purchased from Fisher Scientific (Waltham, Mass.). Paraformaldehyde was purchased from Electron Microscopy Sciences (Hatfield, Pa.). Monoclonal mouse IgG1 anti-vimentin (V9) (catalogue #V6389), 4′,6-Diamidino-2-phenylindole dihydrochloride (DAPI), 10× phosphate buffered saline (PBS), 50×Denhardt's solution, Triton X, poly(ethylene glycol) diacrylate (PEGDA) (Mn=575), N-methyldiethanolamine (MDEA), and 1-vinyl-2-pyrrolidinone were purchased from Sigma Aldrich (Saint Louis, Mo.). Streptavidin-eosin (SA-eosin) is prepared as described previously (Hansen et al. 2008, Biomacromolecules 9:355-362). Water was purified using a Milli-Q system. The isolation of the human endothelial colony forming cells used in these experiments has been described previously (Baker et al. 2009, Am J Respir Crit. Care Med 180:454-461). The fibroblasts were isolated from human umbilical cord out-growth cells. The endothelial cells were positive for eNOS (BD Biosciences 610297), CD31 (Dako M0823), vWF-8 (Dako A0082), VE-Cadherin (Cayman 160840; Ann Arbor, Mich.), and VEGFR-2 (Santa Cruz SC504; Santa Cruz, Calif.) and displayed a cobble-stone morphology. Fibroblasts were negative for these markers and displayed a spindle-like appearance.

Nanoparticles. The NPs used in this study are yellow/green FluoSpheres (20 nm diameter) and Nile red FluoSpheres (20 nm diameter) purchased from Invitrogen. They are comprised of polystyrene with fluorophores embedded in the interior and they have carboxylate surface-functionalization. The yellow/green NPs have a maximum absorbance at 505 nm, and a maximum emission at 515 nm, which is well matched to SA-Alexa488 (495/519) and SA-FITC (494/518). The Nile red NPs have a maximum absorbance at 535 nm and a maximum emission at 575 nm. Both the yellow/green NPs and the Nile red NPs have diameters of 24 (+/−4 nm), as indicated by Invitrogen.

Immunostaining. Cells in eight-well culture slides were washed with 1×PBS, fixed with 4% paraformaldehyde in PBS for 20-30 minutes, then stored refrigerated in PBS. Staining was conducted 1-30 days post fixation with no observed difference in staining intensity or loss of antigens. Immediately prior to staining, the cells were washed with PBS, permeabilized for 5 min with 0.1% Triton X in PBS, then washed with PBS. Next, the cells were blocked with 2% horse serum in PBSA (0.1% BSA in PBS) for one hour, followed by washing with PBSA. Then, primary antibody in PBSA was applied at the specified dilution for one hour, followed by washing with PBSA. Next, secondary antibody in PBSA was applied at the specified dilution for one hour, followed by washing with PBSA. Finally, the SA species (SA-eosin, SA-FITC, or SA-HRP) was prepared at 10 μg/mL in 1× PBS and 5×Denhardt's solution and applied for 30 minutes, followed by washing with PBS and finally with water. An orbital shaker was used during the washing, permeabilization, blocking, binding and rinsing steps. All PBSA and PBS wash steps consisted of three washes of two minutes each. The water rinse consisted of a single 2 minute rinse. For each staining method and target investigated, negative controls were performed in which the primary antibody was omitted, while all other steps were performed as previously described.

FPBA signal amplification. Cells treated with SA-eosin were contacted with 420 mM PEGDA, 210 mM MDEA, 35 mM 1-vinyl-2-pyrrolidinone, and 0.05 wt % fluorescent NPs in water. For the eight-well culture well slides, 100 μL monomer solution was used per well. Polymerization was initiated by a 20 minute exposure to 30 mW/cm² of light at wavelengths greater than 480 nm, followed by three water washes to remove unreacted monomer and NPs that have not been entrapped in polymer films. Argon flow was employed during polymerization to reduce ambient oxygen which inhibits polymerization. Specifically, the slides were placed in a sealed, clear plastic bag and a needle was used to introduce argon to the bag, with the tank pressure set to approximately 3 psi. Argon was introduced for 5 minutes before the light was turned on and argon flow continued throughout the photopolymerization reaction. Investigation of other polymerization reaction times revealed that times shorter than 20 minutes yielded less intense staining, while longer polymerization times often resulted in excessive evaporation of the monomer solution, leading to non-specific binding of the NPs to the surface. An orbital shaker was used for the washes, but not during the polymerization reaction. The polymer films formed are stable to the wash steps. The light source was an Acticure (Exfo, Canada) high pressure mercury lamp with an in-house internal bandpass filter (350 nm-650 nm) and an external 490 nm longpass filter (Edmund Optics, Barrington, N.J.) positioned at the end of a light guide and a collimating lens. The light intensity was measured using an International Light radiometer (Peabody, Mass.).

TSA signal amplification. Cells treated with SA-HRP underwent a 10 min Alexa488-tyramide labeling reaction following the manufacturer's instructions. Specifically, an Alexa488-tyramide working solution was prepared by diluting the stock solution (provided in the TSA kit) 1:100. For the eight-well culture slides, 100 μL of working solution was used per well. After the 10 minute reaction, three PBS rinses of two minutes each were performed, followed by one two minute water rinse. An orbital shaker was employed during the rinse steps, but not during the labeling reaction with Alexa488-tyramide.

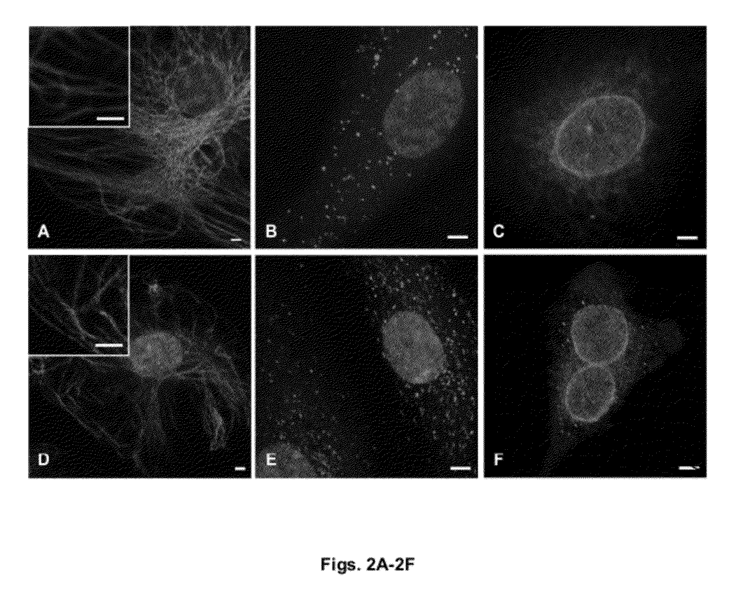

Imaging. Counterstaining of the nucleus was achieved by applying 2 mg/mL DAPI in water for three min, followed by two rinses with water. Cells stained by SA-Alexa488 or by TSA were mounted with Vectashield or Hydromount mounting mediums. Cells stained by FPBA were imaged without mounting medium (simply placing a cover glass on top of the dry slide). Vectashield and Hydromount were investigated for use with FPBA and were found to be incompatible, resulting in almost complete signal loss, likely as a result of swelling the polystyrene NPs which subsequently extracted the encapsulated fluorophores. Nonetheless, appropriate images were achieved, even in the absence of any mounting medium. It was determined water is an appropriate mounting medium for FPBA and was used to take the image in FIG. 2C. Epifluorescence microscopy was performed with a Nikon TE 2000 using a 20× objective (Japan) and an X-Cite 120 lamp (Exfo, Canada). Confocal scanning laser microscopy (CLSM) was performed with a Zeiss LSM 510 instrument (Germany) with a 40× oil objective or 63× oil objective, as indicated.

Fluorescence Quantification. Quantification of fluorescence intensities was done using ImageJ 1.40 g (NIH). Error bars represent standard error. P-values were determined for one-tailed student's t-test. For each staining condition quantified, measurements were made for all cells in four images obtained from at least two separate staining sessions. Non-specific staining in cells imaged for NPC was quantified by measuring the fluorescence in an area equal to the size of the nucleus, but immediately to the left of the nucleus.

Double immunostaining with FPBA. Binding reactions to stain NPC were performed as described previously, except that blocking and antibody-binding steps were only 45 minutes rather than one hour, and SA-eosin was applied to the surface for only 20 minutes rather than 30 minutes. The first round of polymerization employed Nile red NPs. Immediately following the first polymerization step, the cells were blocked again and binding reactions were performed to stain vimentin, utilizing 45 minute reaction times for the blocking and antibody binding steps, and 20 minutes for the SA-eosin binding step. The second polymerization utilized yellow/green NPs. The two polymerization steps incorporated NPs of different colors to enable facile discrimination of the independent responses. Two negative controls were performed: 1) NPC primary antibody was omitted, while all other steps were performed the same and 2) alternatively, vimentin primary antibody was omitted, while all other steps were performed as usual. Each of the negative controls were imaged for detection of both Nile red NPs and yellow/green NPs.

Photostability. Epifluorescence microscopy was performed as above, except the excitation source was an Acticure (Exfo, Canada) high pressure mercury lamp with an in-house internal bandpass filter (350 nm-650 nm). This lamp is designed to achieve an exceptionally stable light intensity. The slides were continuously illuminated while images were taken at the indicated times. All images were taken without mounting medium present to assure a valid comparison, as mounting medium can alter the photostability of the dye (Wu et al. 2003, Nat Biotechnol 21:41-46). A cover glass was placed over the dry slide.

### Results

### Comparison of FPBA and Streptavidin-Alexa488 (SA-Alexa488) for Staining a Variety of Cellular Antigens

By generating a fluorescent film in response to biorecognition, FPBA immobilizes a significantly greater number of fluors to the surface as compared to staining with probes that are directly labeled with fluorophores; however, the generation of a polymer film with a finite thickness brings into question the spatial resolution of the stain and the types of structures that may be imaged. To verify that FPBA achieves similar staining patterns as fluor-labeled probes, staining of various antigens was performed using biotinylated secondary antibodies and either FPBA or SA-Alexa488 to generate a fluorescent signal. SA-Alexa488 was selected for comparison because Alexa488 absorbance is well-matched to the photoluminescent properties of the yellow/green NPs used for FPBA. Moreover, since streptavidin-eosin (SA-eosin) is used for FPBA, SA-Alexa488, as opposed to a fluorescent antibody, was chosen for comparisons such that both methods employ a similar streptavidin-biotin approach. FIG. 2 demonstrates that the two staining methods yielded similar staining patterns and resolution for a variety of fine cellular structures including filamentous vimentin in the cytoplasm of fibroblasts, the nuclear pore complex proteins (NPC) located in the nuclear envelope, and von Willebrand Factor (vWF) which is present in the cytoplasm of endothelial cells, often concentrated in granules. For both FPBA and SA-Alexa488, staining of vimentin yielded images in which many of the filaments were measured to be 500 nm wide, with very few, if any, measuring less than 200 nm wide. Since the fluorescent polymer is layered on top of the cellular feature that is being stained, and since film formation occurs uniformly as it moves away from the filament surface, the polymer film itself must be less than at least 250 nm thick. This result is consistent with previously published microarray format experiments in which films formed from surface immobilized SA-eosin under similar monomer and polymerization conditions were found to be five to 200 nm thick (Avens, et al. 2008, Polymer 49:4762-4768). In the case of NPC immunostaining, both FPBA and SA-Alexa488 yielded a distinct ring structure around the nucleus, in which the ring was measured to be approximately 500 nm thick. In NPC imaging, both FPBA and SA-Alexa488 resulted in non-specific staining around the nucleus which is attributable to non-specific binding of the primary antibody. For each of these staining targets, negative controls were performed in which the primary antibody was omitted and all other steps were performed as usual. Under matched imaging conditions, the negative controls showed no visible staining. Additionally, it is of note that the FPBA confocal scanning laser microscopy (CSLM) images in FIGS. 2A and 2B were obtained without mounting medium, simply setting a cover glass on the dry slide, while FIG. 2C utilized water as a mounting medium. The SA-Alexa488 CLSM images (FIGS. 2D-F), on the other hand, were obtained using Hydromount mounting medium. The use of no mounting medium or water as a mounting medium is anticipated to negatively impact the fluorescence intensity and the resolution compared to the use of Hydromount. It is expected that development or identification of a more suitable mounting medium for FPBA will yield higher resolution FPBA images. Overall, these results verify that FPBA achieves similar staining patterns to those observed when utilizing a fluor-labeled probe.

### Comparison of the Relative Sensitivity of FPBA, SA-Alexa488, and TSA.

To assess the magnitude of the signal amplification afforded by FPBA, this method was directly compared to the use of a fluor-labeled probe (SA-Alexa488) for the immunofluorescent imaging of NPC in fibroblast cells at decreasing primary antibody concentrations. Reduced primary antibody concentrations generally result in fewer biorecognition events, thereby enabling a systematic comparison of the relative sensitivities of each of the staining methodologies. Further, to benchmark FPBA relative to a commercially available enzymatic signal amplification approach, NPC staining was also performed by TSA utilizing SA-HRP and Alexa488-tyramide. All three staining approaches employed fluorophores that have similar excitation and emission spectra as discussed in the Methods section, such that each staining approach is equally well suited for the epifluorescence filter set that was utilized. In addition, all three methods employed the streptavidin-biotin approach.

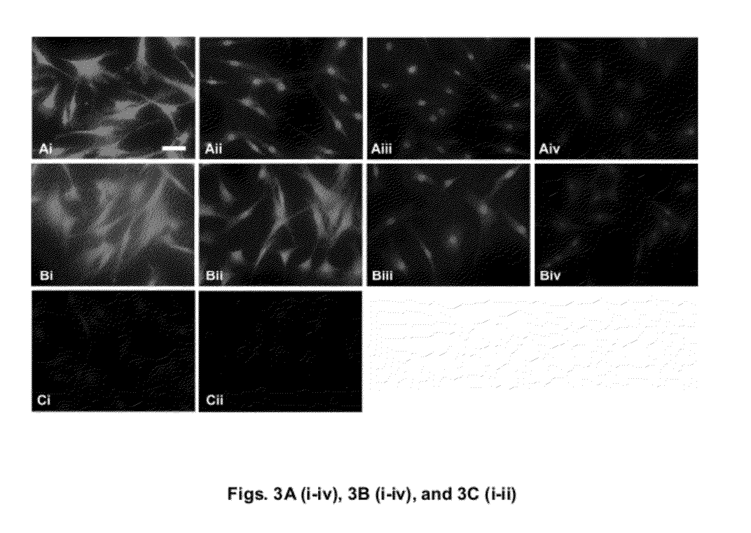

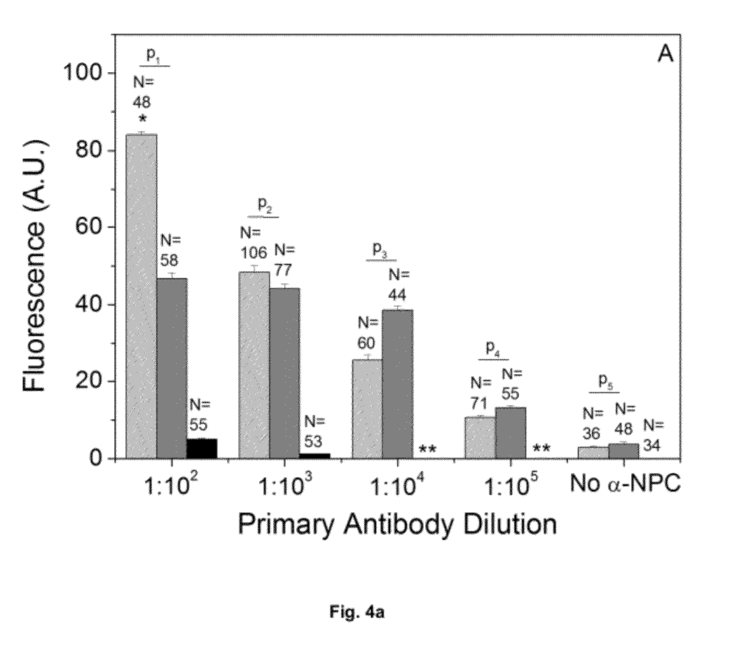

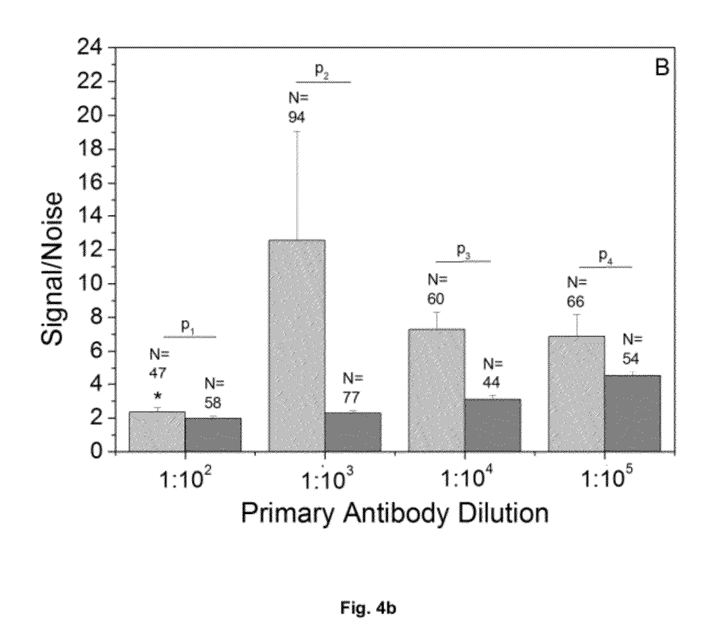

FIG. 3 shows the imaging results for FPBA, SA-Alexa488, and TSA at four different anti-NPC primary antibody concentrations, using matched camera and image settings (four second exposure, no binning of pixels). Additionally, the fluorescence intensity of NPC staining was quantified and is depicted in FIG. 4A. Although a four second exposure resulted in saturated fluorescence in the cells that were treated with 1:100 primary antibody dilution and stained by FPBA, for the purpose of making quantitative comparisons, the same exposure time is used in all conditions. Also, epifluorescence microscopy, as opposed to CLSM, was used for FIGS. 3 and 4 because of its ease of signal quantification. Epifluorescence microscopy captures fluorescence emitted from all planes of the image, readily enabling quantification of an overall fluorescence emission. On the other hand, CLSM images only a single slice of the specimen at a time, making quantification more complicated as it depends on the depth of the slices, the number of slices, and the space between the slices. At the highest NPC antibody concentration (1:100 dilution), FPBA signal was saturated while SA-Alexa488 showed no visually detectable staining, though quantification did reveal a small amount of fluorescence signal (FIGS. 3A,C and 4A). At 1:1000 anti-NPC dilution, the FPBA signal was 48, whereas the SA-Alexa488 signal was only 1.2, below the camera's linear detection range. Based on these measurements, it is concluded that FPBA enables a greater than 40-fold improvement in fluorescence intensity compared to SA-Alexa488.

Comparison of FPBA with TSA revealed that both methods generated detectable NPC staining across all four primary antibody concentrations investigated. Both methods enabled NPC staining down to 1:100,000 primary antibody dilution, with fluorescence intensities of 11 (+/−1) for FPBA and 13 (+/−1) for TSA (FIGS. 3A,B and 4A). Likewise, both methods displayed a small amount of non-specific nuclear staining in the absence of primary antibody, possibly due to non-specific binding of secondary antibody. At the highest antibody concentration (1:100 dilution), FPBA generated saturated signals, indicating that the true average signal is actually higher than 84; in contrast, the TSA signal was only 47. Indeed, the TSA signal appeared to plateau as the primary antibody concentration was increased, whereas FPBA continued to increase with increasing primary antibody concentration. Notably, decreasing the exposure time from four seconds to 500 ms prevented signal saturation of the FPBA-treated cells and enabled more clear visualization of the NPC staining; however, for the purpose of this comparative quantitative study, exposure times were not varied.

The TSA NPC stain in FIG. 3 is observed to be less localized than the FPBA stain obtained with the same primary antibody dilution. To quantify stain localization, non-specific staining “noise” was assessed for each cell by measuring the fluorescence in an area equal to the size of the nucleus, but immediately adjacent to the nucleus. The specific NPC fluorescence signal was then divided by the noise to generate “signal/noise” values. FPBA was found to have signal/noise values ranging from 6.9 to 13 for dilutions greater than 1:100 while for the1:100 primary antibody dilution, the positive signal was truncated due to saturation of the camera's detector (FIG. 4B). In contrast, TSA displayed signal to noise values ranging from 2.0 to 4.5, with the best signal/noise occurring at 1:100,000 primary antibody dilution, where specific signal is the lowest. Some of the non-specific signal adjacent to the nucleus likely arises from non-specific binding of the primary antibody; however, differences observed between signal/noise in FPBA and TSA are likely attributable to differences in signal localization between the two methods. The fact that the FPBA tends to have higher signal/noise, suggests that FPBA yields better signal localization than TSA. Additionally, it is of note that no mounting medium was used on the FPBA slides for the data utilized in FIGS. 3 and 4, whereas Vectashield mounting medium was employed for the TSA and SA-Alexa488 samples. As mounting medium is intended to improve signal intensity and image resolution, it is expected that identification of a suitable mounting medium for FPBA would yield images with further increased signal intensities and possibly better signal/noise as a consequence of improved image resolution.

### Multicolor Staining of Two Antigens by Sequential FPBA Reactions

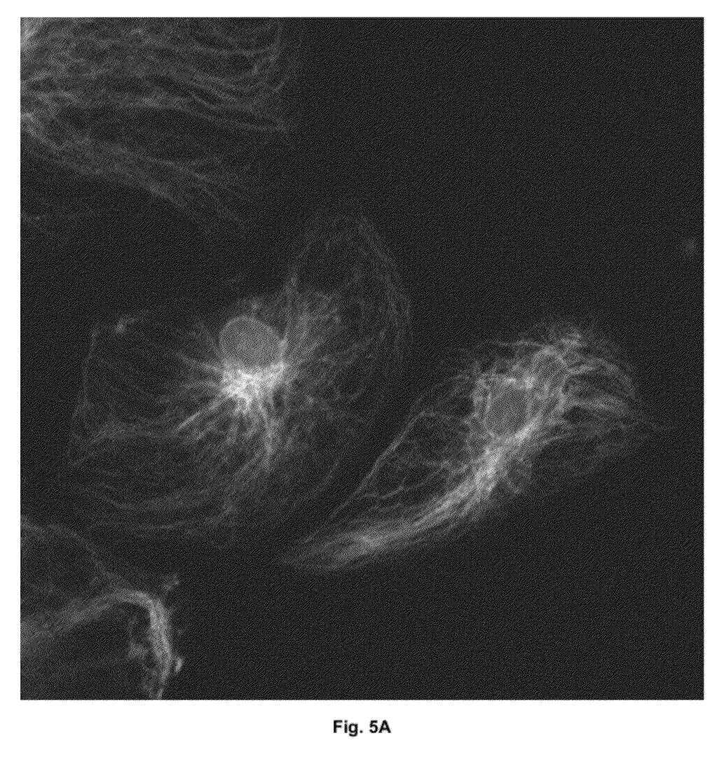

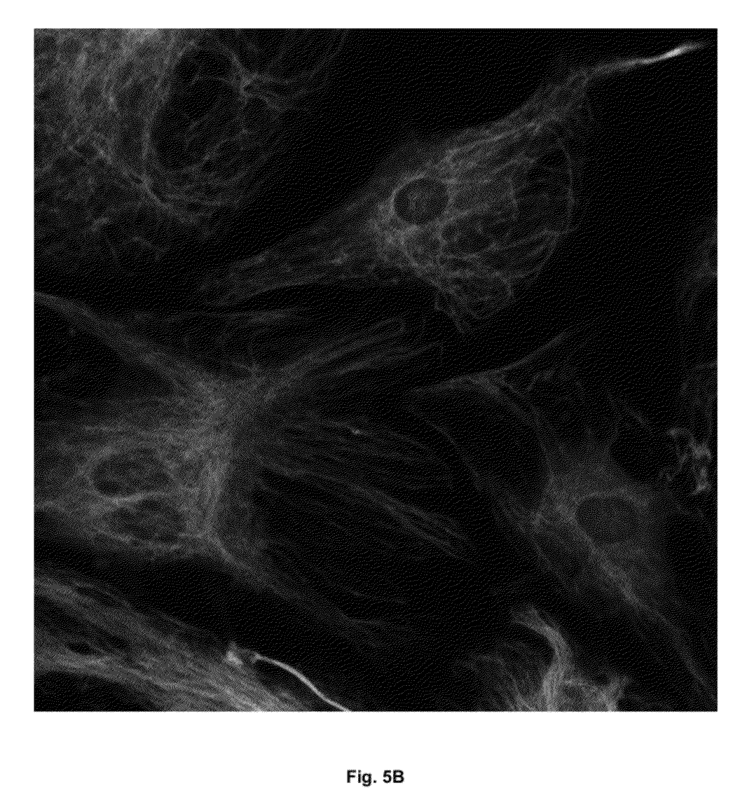

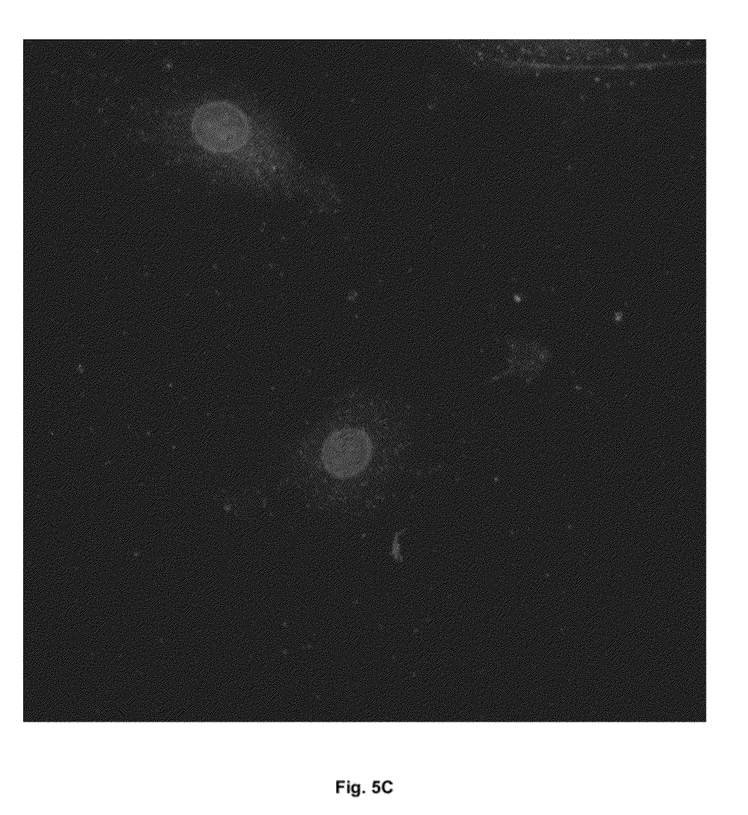

It was of interest to determine whether multiple antigenic targets in the same cell could be stained by sequential rounds of FPBA. FIG. 5 demonstrates successful sequential immunostaining of two targets by FPBA in endothelial cells. In the first round of staining, NPC was stained by FPBA utilizing Nile red NPs. Immediately following this first staining reaction, the cells were stained for vimentin by a second round of FPBA utilizing yellow/green NPs. Two negative controls were performed: 1) the primary antibody against NPC was omitted (FIG. 5B); and 2) the primary antibody against vimentin was omitted (FIG. 5C). Each negative control was imaged for detection of both Nile red and yellow/green NPs. The successful vimentin staining depicted in FIGS. 5A and 5B verifies that the polymerization reaction is mild enough that antigenic sites are still intact and available for antibody recognition and immunostaining after the first round of FPBA. Further, FIGS. 5A and 5C reveal that the polymer films from the first round of staining are not displaced or rendered non-fluorescent during the second round of staining. An additional anticipated challenge of two-round FPBA is that eosin photoinitiators remaining from the first round of polymerization might initiate polymerization during the second round, yielding non-specific staining. FIG. 5C demonstrates that no non-specific green staining occurred around the nucleus, verifying that eosin from the previous round of FPBA did not interfere with the second FPBA detection reaction.

### Evaluation of the Photostability of Fluorescence Signals Generated by FPBA.

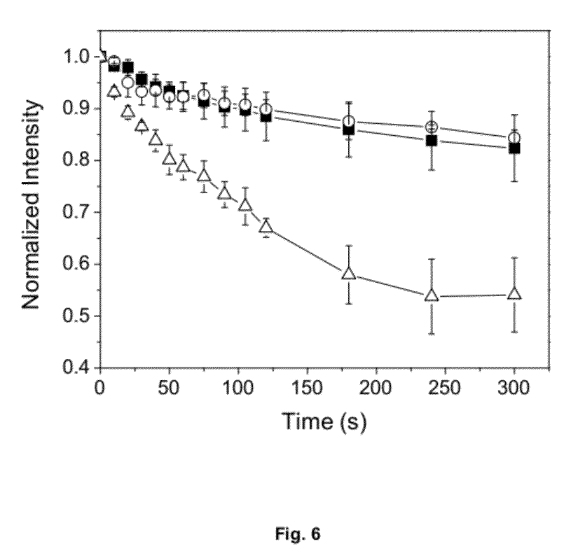

Generally, photobleaching during composing images and adjusting acquisition settings causes the intensity of the fluorescent stain to decrease, resulting in irreproducible results and loss of weakly fluorescent signals. Since FPBA utilizes fluorescent NPs which are reported to be more photostable than free fluors (Mayr et al. 2009, J Fluoresc 19:303-310), it is expected that FPBA will yield a more photostable fluorescent stain compared to other staining approaches. On the other hand, the fact that the photopolymerization step of FPBA requires the fluorescent NPs to experience a 20 minute exposure to intense light calls into question whether the NP fluorescence will be as photostable as has been reported previously. To evaluate this question, the photostability of FPBA with yellow/green NPs was compared to staining with SA-Alexa488 and SA-FITC in the context of imaging vimentin in fibroblast cells. All images were taken without mounting medium present to assure a valid comparison, as mounting medium can alter the photostability of the dye (Wu et al. 2003, Nat Biotechnol 21:41-46). Alexa488 is well-known for being highly photostable, while FITC is generally acknowledged as being quite unstable. FPBA and SA-Alexa488 showed similar rates of photobleaching, while staining with SA-FITC, resulted in more rapid photobleaching (FIG. 6). After five minutes of illumination, the fluorescence from FPBA and SA-Alexa488 remained at greater than 80% of its initial value, whereas the fluorescence from SA-FITC had decreased to nearly half its original intensity. Though FPBA shows similar photobleaching rates as SA-Alexa488, it should be noted that FPBA generates many-fold higher signal intensities than SA-Alexa488 or SA-FITC, such that a nearly 10-fold longer exposure time is required for imaging the cells stained by the latter methods.

### Discussion

The results presented here highlight FPBA as uniquely advantageous in providing highly sensitive and specific detection accompanied by extremely strong fluorescent signals without suffering from the disadvantages associated with enzymatic amplification. FPBA was demonstrated to enable similar resolution and staining patterns as those achieved with SA-Alexa488 (FIG. 2). The observation that staining cellular features with FPBA generates films at least as thin as 250 nm is consistent with previously published microarray-format experiments in which polymer films formed from surface immobilized SA-eosin under similar conditions were found to be five to 200 nm thick (Avens, et al. 2008, Polymer 49:4762-4768).

The improvements in signal intensity and detection sensitivity afforded by FPBA relative to staining with SA-Alexa488 are similar to those achieved by the enzymatic TSA approach (FIGS. 3 and 4). Both FPBA and TSA enabled use of reduced primary antibody concentrations which are associated with a significant cost savings as primary antibodies are often rare and costly, frequently constituting the most expensive component of the staining reagents. Additionally, as TSA is known to be a highly sensitive amplification technique capable of visualizing single binding events (Schmidt et al. 1997, J Histochem Cytochem 45:365-373), these results suggest that FPBA will likewise prove to be suitable for detection of very low abundance antigens. Finally, although SA-Alexa488 staining of NPC was visualized easily using CLSM, only extremely faint staining was achieved using epifluorescence imaging. In contrast, FPBA and TSA both enabled facile epifluorescence imaging of NPC. As epifluorescence microscopy is much less expensive and more widely available than CSLM, signal amplification methods that permit the use of more affordable and accessible instrumentation are of value.

Although both FPBA and TSA are able to achieve similar fluorescence gains and sensitivities, FIGS. 3 and 4 indicate that FPBA tends to have better signal/noise and better signal localization. One explanation for the enhanced localization of FPBA relative to TSA is that FPBA signal amplification occurs by formation of a localized, crosslinked film at the biorecognition site which hinders initiating radicals from diffusing far from the site of biorecognition. The TSA process, on the other hand, results in enzymatically activated tyramide-Alexa488 molecules that are free to diffuse away from the biorecognition site before covalently attaching to cellular structures. Previously published studies have noted that TSA often requires prolonged meticulous adjustment of the primary antibody concentration or the enzyme reaction time to mitigate diffusion-related loss of signal localization; however, these measures also reduce the signal amplification, ultimately limiting the sensitivity of the TSA method (Kerstens et al. 1995, J Histochem Cytochem 43:347-352; Wiedorn et al. 1999, Histochem Cell Biol 111:89-95). Likewise, it is expected that shorter polymerization times may improve FPBA signal localization by generating thinner polymer films, though signal intensity would likely be reduced as well. A second advantage of FPBA is that it is non-enzymatic, and therefore is not expected to be impacted by endogenous enzymes. A significant problem with the TSA approach, which has been reported to be difficult and sometimes infeasible to completely eliminate, is endogenous peroxidase activity of some cell types that results in non-specific staining (Hunyady et al. 1996, Histochem. Cell Biol. 106, 447-456; Ishii et al. 2004, FEMS Microbiol Ecol 50:203-212; Pavlekovic et al. 2009, J Microbiol Methods 78:119-126). Thus, FPBA generates similarly high levels of signal amplification as TSA, yet tends to yield better stain localization and is not expected to be impacted by endogenous enzymes.

The capability of FPBA to immunostain multiple targets was demonstrated by successfully staining NPC and vimentin via sequential immunostaining reactions. Negative controls confirmed that the polymerization conditions do not excessively damage antigens and that eosin from the first staining reaction does not initiate non-specific polymerization in subsequent FPBA steps. The eosin from the first reaction was likely either consumed or trapped within the polymer film during the first photopolymerization reaction. The polymer film formed in the first round of FPBA acts as a diffusive barrier, reducing the transport of MDEA coinitiator and monomer to eosin, thereby hindering additional polymerization. It should also be noted that the capability of the fluorescent polymer film to act as a diffusion barrier may impact the second round of staining negatively. For example, after FPBA is used to stain NPC, it might become more difficult to stain antigens that are colocalized on the nuclear envelope or sub-nuclear antigens that require antibodies to pass through the nuclear envelope. Though this potential limitation has not yet been investigated experimentally, if it does indeed prove to be problematic, simply selecting an appropriate order of antigen staining would circumvent these complications in most instances. Combining FPBA with other fluorescent staining methods for multi-antigen staining is also expected to be successful, provided that FPBA is the first method employed, as the illumination during the photopolymerization reaction may excessively photobleach fluors that are not embedded within NPs.

The photostability of FPBA staining was found to be better than SA-FITC and equal to SA-Alexa488, while achieving dramatically brighter fluorescence intensities than either SA-Alexa488 or SA-FITC. Though Alexa488 is well-known for being an extremely photostable fluor, it is noteworthy that FPBA is not more photostable than SA-Alexa488, as the former method utilizes fluorescent NPs which have been reported to be more photostable than Alexa488 (Mayr et al. 2009, J Fluoresc 19:303-310). It is likely that some aspect of FPBA staining such as the illumination during photopolymerization or a component of the monomer formulation may negatively impact photostability during imaging, thereby counteracting any benefits associated with the fluorescent NPs. Nonetheless, it is observed that FPBA has photostability equivalent to Alexa488 while enabling many fold brighter fluorescence intensity. By providing both good photostability and intense fluorescence signals, FPBA facilitates reproducible imaging and helps prevent loss of weak signals during image acquisition.

In conclusion, FPBA was shown to be highly sensitive, generating a greater than 40-fold increase in fluorescent signal compared to SA-Alexa488. Compared to the TSA enzymatic approach, FPBA appears to have similar sensitivity as both methods enable the use of similarly low primary antibody concentrations for detection of NPC. The results also suggest that FPBA improves upon TSA by achieving better signal localization. Further, since FPBA is non-enzymatic, the method is not expected to be affected by endogenous enzymes as occurs with enzymatic amplification methods. Finally, FPBA provides photostability equivalent to Alexa488, and the capability for multicolor staining of multiple cellular targets. The results presented here establish FPBA as providing a combination of advantageous attributes not currently available by any other immunofluorescent cell staining method. See also Avens et al. (2011), Journal of Histochemistry and Cytochemistry 59 (1) 76-87, hereby incorporated by reference in its entirety.

### Example 2

Incorporation of nanoparticles (NPs) into polymer films represents a valuable strategy for achieving a variety of desirable physical, optical, mechanical, and electrical attributes. Here, we describe and characterize the creation of highly fluorescent polymer films by entrapment of fluorescent NPs into polymer matrices through surface-mediated eosin photoinitiation reactions. Performing surface-mediated polymerizations with NPs combines the benefits of a covalently anchored film with the unique material properties afforded by NPs. The effects of monomer type, crosslinker content, NP size, and NP surface chemistry were investigated to determine their impact on the relative amount of NPs entrapped in the surface-bound films. The density of entrapped NPs was increased up to six-fold by decreasing the NP diameter. Increasing the crosslinking agent concentration enabled a greater than two-fold increase in the amount of NPs entrapped. Additionally, the monomer chemistry played a significant role as poly(ethylene glycol) diacrylate (PEGDA)-based monomer formulations entrapped a ten-fold higher density of carboxy-functionalized NPs than did acrylamide/bisacrylamide formulations, though the latter formulations ultimately immobilized more fluorophores by generating thicker films. In the context of a polymerization-based microarray biodetection platform, these findings enabled tailoring of the monomer and NP selection to yield a 200-fold improvement in sensitivity from 31 (+/−1) to 0.16 (+/−0.01) biotinylated target molecules per μm². Similarly, in polymerization-based cell staining applications, appropriate monomer and NP selection enabled facile visualization of microscale, sub-cellular features. Careful consideration of monomer and NP selection is important to achieve the desired properties in applications that employ surface-mediated polymerization to entrap NPs.

### Introduction

Polymeric nanocomposites are of significant interest, heralded for achieving further enhancements in material properties as compared to microcomposite approaches (Allegra et al. 2008, Prog Polym Sci 33:683-731). Incorporation of nanomaterials in polymeric matrices has been employed in a variety of applications including generation of materials with enhanced optical, magnetic, electrical, thermal, and mechanical properties (Balazs et al. 2006 Science 314:1107-1110; Dhibar et al. 2009, J Appl Polym Sci 113:3012-3018; Durán et al. 2008, J Pharm Sci 97:2948-2983; Luo et al. 2009, J Phys Chem C 113:9406-9411; Verma et al. 2009, Polym Compos 30:490-496). Additionally, nanocomposite coatings have been developed that achieve various desirable surface modifications including superhydrophobicity (Xu et al. 2009, Colloids Surf: Physiochem Eng Aspects 341:21-26), corrosion resistance (Olad and Rashidzadeh 2008, Prog Org Coat 62:293-298), reduced gloss (Balan et al. 2008, Prog Org Coat 62:351-357), and enhanced mechanical strength (Zhu, 2007, J Mater Sci 42:545-550).

Recently, the use of fluorescent nanocomposite films has been reported for sensitive and photostable biodetection in microarrays and immunocytochemical staining (Avens and Bowman 2010, Acta Biomater 6:83-89; Avens et al., J Histochem Cytochem, vol. 59 no. 1, 76-87; Hansen et al. 2008, Anal Bioanal Chem 392:167-175). This approach, coined “fluorescent polymerization-based amplification” (FPBA), utilizes biological probes selectively labeled with eosin photoinitiators. After the probe has bound its target on the microarray or within the cell, a solution of monomer, coinitiator and fluorescent nanoparticles is applied and the test surface is exposed to light of wavelengths greater than 480 nm to initiate polymerization. This approach results in the formation of crosslinked polymer films specifically in regions of the microarray or cell where the probe has bound its target. As the polymer film forms, it entraps the fluorescent nanoparticles, rendering the film highly fluorescent. In this manner, the presence and localization of a biological target is evidenced by a highly fluorescent nanoscale polymer film. During the development of FPBA, other strategies for generating fluorescent films were considered, such as using monomers covalently attached to organic fluorophores; however, the approach employed was unsuitable because the organic fluorophores themselves non-specifically initiated polymerization and/or underwent extensive photobleaching (Avens and Bowman, 2010, Acta Biomater 6:83-89, Acta Biomater 6:83-89). To overcome these challenges, polystyrene nanoparticles with fluorophores embedded in the interior were used to enable facile formation of fluorescent films with fewer problematic side reactions between the monomer and the fluorophores. FPBA has been demonstrated to yield approximately 100-fold brighter signals and a 100-fold improvement in detection sensitivity compared to using a molecular fluor-labeled probe (Avens and Bowman 2010, Acta Biomater 6:83-89, Avens H J et al., J Histochem Cytochem, vol. 59 no. 1, 76-87). Additionally, in cell staining applications, FPBA compared favorably to the highly sensitive tyramide signal amplification (TSA) approach which relies on peroxidase enzymes. Moreover, while both TSA and FPBA generated similarly intense fluorescent signals, FPBA is advantageous in that it is not affected by endogenous cellular peroxidase enzymes which cause non-specific TSA staining (Avens H J et al., J Histochem Cytochem, vol. 59 no. 1, 76-87).

Though a variety of surface-mediated initiation strategies are suitable for generation of NP-polymer composite films, the method presented here employs surface-immobilized eosin as a type II photoinitiator for polymerization. Upon photoexcitation with visible light, eosin undergoes energy, charge, and electron transfer with a coinitiator, commonly a tertiary amine such as N-methyldiethanolamine (MDEA), to yield a relatively stable eosin radical and an MDEA radical capable of initiating polymerization (Avens and Bowman 2009, J Polym Sci Part A: Polym Chemistry 47:6083-6094). Despite the fact that the initiating MDEA radicals are not tethered to the surface, the reaction is still considered surface-mediated because the initiating radicals are generated only at the surface. Covalent attachment of the films to the surface is believed to occur through a variety of chain transfer reactions and other processes including termination of the polymer chains with the eosin radical which is reported to be inactive for initiation, but capable of reacting by termination (Kizilel et al. 2004, Langmuir 20:8652-8658). Eosin has been used for surface-mediated polymerization for an assortment of purposes including modification of nanoparticle surfaces to enhance dispersion (Satoh et al. 2005, J Polym Sci. Part A: Polym Chem 43:600-606), islet cell encapsulation (Cruise et al. 1998, Biotechnol Bioeng 57:655-665), creation of protective surface coatings for arterial walls (An and Hubbell 2000, J Controlled Release 64:205-215), and generation of macroscopically visible and fluorescent films for sensitive signal amplification of biodetection (Hansen et al. 2008, Anal Bioanal Chem 392:167-175; Avens and Bowman 2010, Acta Biomater 6:83-89). The fact that eosin uses visible light which is less damaging to biological systems than UV light, and the observation that eosin is less sensitive to oxygen inhibition than other photoinitiators (Avens and Bowman 2009, J Polym Sci Part A: Polym Chemistry 47:6083-6094) makes eosin an excellent choice for numerous surface modification applications. Importantly, photoinitiation, as opposed to other initiation strategies, readily enables precise spatial and temporal control of the polymerization process, factors which are often critical for achieving the desired material properties. (Bowman and Kloxin 2008, AIChE J 54:2775-2795). Though photoinitiation is the focus of this work, the NP incorporation results presented here are expected to be generalizable to other surface-mediated polymerization processes including other polymerization-based detection strategies (He et al. 2008, Anal Chem 80:3633-3639; Lou et al. 2005, Anal Chem 77:4698-4705).

For FPBA and other applications that employ surface-mediated polymerization with NPs, the NP and monomer attributes can be optimized to achieve the desired polymer properties. To more aptly employ this type of nanocomposite film formation for biodetection and other applications, a detailed study is presented here investigating the effects of NP size, NP surface functionality, monomer type and crosslinking agent content on the fluorescence and thickness of the polymer films. For FPBA, it is generally desired to incorporate as high a density of NPs as possible to maximize the subsequent fluorescent signal emanating from the surface. It is hypothesized that chemical and mechanical interactions occurring at the polymer-monomer-NP interface during the polymerization strongly influence the successful entrapment of NPs into the growing polymer matrix. To identify trends that are relevant across a broad range of initiation rates, surface-mediated polymerizations were carried out using a microarray format in which each row of spots comprises a different eosin surface density, such that a wide range of eosin photoinitiator densities were evaluated simultaneously. Reactions were performed with both an acrylamide-based monomer formulation and a poly(ethylene glycol) (PEG) acrylate-based formulation where the acrylamide polymerization is known to occur more rapidly and the PEG-based formulation is more hydrophilic. Trends that are common between these two monomer formulations are expected to be pertinent to other monomer types as well, while differences are correlated to the differences in the chemistry and reaction behavior of the formulations.

The significance of the trends presented here was investigated in the context of two specific applications. Firstly, it was shown that through selection of the appropriate monomer type, crosslinker content and NP size one is able to significantly increase the absolute amount of fluorescent NPs immobilized on the surface, yielding a 200-fold improvement in detection sensitivity in a polymerization-based microarray biodetection platform compared to using sub-optimal reaction conditions. Secondly, by selecting parameters to optimize the NP density entrapped in the film, rather than just the total NP amount immobilized to the surface, it was possible to create highly fluorescent, yet thin and spatially restricted films suitable for visualization of microscale sub-cellular features in polymerization-based fluorescent immunostaining of cells. The principles outlined here are expected to be useful for tailoring monomer and NP attributes to achieve desired properties in additional applications in which NPs are being entrapped in surface-mediated polymerizations, including in the context of different polymer matrices and different types of NPs.

### Experimental

Materials. Poly(ethylene glycol) diacrylate, Mn=575 (PEGDA), Poly(ethylene glycol) acrylate, Mn=375 (PEGA), 40 wt % acrylamide in water, N,N′ methylenebisacrylamide (bisacrylamide), N-methyldiethanolamine (MDEA), vinyl pyrrolidone (VP), 4′,6-Diamidino-2-phenylindole dihydrochloride (DAPI), streptavidin (SA), biotin-labeled anti-goat IgG (bi-antibody) produced in rabbit, bovine serum albumin (BSA), and 10×PBS were purchased from Sigma Aldrich. Eosin-isothiocyanate, carboxylate-functionalized yellow/green NPs (20, 40, 100, and 200 nm diameter FluoSpheres), carboxylate-functionalized dark red NPs (20, 40, and 200 nm diameter FluoSpheres), amine-functionalized yellow/green NPs (200 nm diameter FluoSpheres), and carboxylate-functionalized crimson NPs (20 nm diameter FluoSpheres) were purchased from Invitrogen. Table 1 provides the actual NP diameters and relative quantum yields as provided by Invitrogen for each of the NPs utilized. TEM images of FluoSpheres have been published previously (Kobayashi et al. 2004, Colloids and Surfaces A: Physicochem Eng Aspects 242:47-52; Lansalot et al. 2005, Colloid Polym Sci 283:1267-1277; Popielarski et al. 2005, Bioconjugate Chem 16:1063-1070). Epoxy-functionalized glass slides (SuperEpoxy 2), 2× Protein Printing Buffer and solid printing pins were purchased from Telechem International, Inc. Biotin-labeled anti-mouse IgG produced in goat was purchased from Pierce Biotechnology while anti-nuclear pore complex proteins IgG (anti-NPC) produced in mouse was purchased from Covance. Streptavidin-eosin conjugate (SA-eosin) was prepared in-house as described previously (Hansen et al. 2008, Biomacromolecules 9:355-362) by reacting eosin isothiocyanate with amines on the protein's surface. A Cy3 Scanner Calibration slide was purchased from Full Moon BioSystems. Water was purified using a Milli-Q system. The cells used for immunofluorescent imaging are endothelial colony forming cells whose isolation has been described previously (Baker et al. 2009, Am J Respir Crit. Care Med 180:454-461).

Compare Photoluminescence of NPs in solution and in polymer. To verify that encapsulation in polymer films does not dramatically alter NP photoluminescence, an Ocean Optics USB4000-FL detector was used to measure emission spectra of free 20 nm yellow/green NPs in water (0.05 wt % NPs) as well as NPs that had been entrapped in a polyacrylamide film analogous to the ones formed on the surfaces. The excitation source was a 370 nm CrystaLaser. The samples were prepared in 1 cm wide UV/VIS cuvettes.

Preparation of Eosin Dilution Chips. Sa-Eosin was Printed onto Epoxy-functionalized glass slides using a VersArray Chip Writer™ Pro (Bio-Rad) with approximately 45% humidity and a solid pin yielding spots of approximately 500 μm diameter. 9×5 arrays of spots were prepared containing five replicate spots of 8 decreasing eosin concentrations, and a 9^{th} row with no eosin that served as a negative control suitable for evaluating non-specific polymerization. The highest print concentration was 0.27 mg/mL SA-eosin (12 μM eosin, 1.3 eosin per streptavidin) in a final concentration of 1× printing buffer. Lower print concentrations were prepared by serial 1:3 dilutions into 1× print buffer with unmodified streptavidin such that all spots were printed with a total protein concentration of 0.27 mg/mL. Unbound SA-eosin was removed by three fifteen minute rinses in water with rapid rocking. Since eosin is a fluorophore, which also serves as the initiator, surface density was characterized with an Agilent Technologies Microarray Scanner. A Cy3 Scanner Calibration slide was used to convert fluorescence readings into surface density of eosin. The slides were then polymerized as described subsequently.

Preparation of Biotin Dilution Chips. Microarray Printing as Described for Sa-eosin was likewise performed to create slides with a dilution series of biotin-a-goat IgG in a 9×5 array containing five replicate spots of 8 decreasing antibody concentrations, and a 9^{th} row with no antibody that served as a negative control suitable for evaluating non-specific polymerization. The highest print concentration was 85 μg/mL biotin-a-goat IgG in 1× printing buffer and 0.5 mg/mL BSA. Lower print concentrations were prepared by serial 1:3 dilutions into 1× print buffer with 0.5 mg/mL BSA, and the 9^{th} row contained only 0.5 mg/mL BSA. The slides were stored unprocessed until they were used for binding and detection reactions. The surface concentration of bound antibodies was estimated as described previously (Avens and Bowman 2010, Acta Biomater 6:83-89).

Binding reactions with the biotin dilution chips. The biotin-a-goat IgG dilution chips were rinsed with water to remove unbound antibody, then the slides were incubated with 10 mg/mL BSA in 1× PBS for 1 hour, followed by three 2 minute rinses in PBS with rapid rocking. The dilution chips were then reacted 1 hour with 1 μg/mL SA-eosin, using Chip Clips from Whatman to form wells on the chips and gentle rocking was used, followed by three 2 minute rinses in PBS with rapid rocking and one rinse in water. The slides were then polymerized as described subsequently.

Cell staining procedures. Cells in micro-chamber slides were rinsed with 1× PBS then fixed with 4% paraformaldehyde for 15 minutes. The slides were then stored in 1× PBS and refrigerated until staining. At the time of staining, slides were rinsed 3 min with 1× PBS, permeabilized with 0.1% Triton-X in PBS for 5 minutes, then rinsed three times with PBS. Next, the slides were blocked for 1 hour with 2% horse serum and 0.1% BSA in PBS, and then rinsed three times with 0.1% BSA in PBS (PBSA). Anti-NPC IgG at a dilution of 1:1000 in PBSA was applied for 1 hour, followed by three rinses with PBSA. Next, biotin-anti-mouse IgG was applied for 1 hour at a dilution of 1:400 in PBSA, followed by three rinses with PBSA. The slides were then contacted with 10 μg/mL SA-eosin for 30 minutes, followed by three rinses with PBS and one rinse with water. Polymerization reactions were then carried out as described in the following section. After polymerization, the nuclei of the cells were stained 2 minutes with 1 μg/mL DAPI in water.

Polymerization reactions. The microarray or cell slide surfaces were contacted with the desired monomer solution. For five minutes prior to and throughout the entire light exposure, the slides were placed in a plastic bag with argon flow to reduce oxygen in the atmosphere. The light source was an Acticure (Exfo) high pressure mercury lamp with an in-house internal bandpass filter (350 nm-650 nm) and an external 490 nm longpass filter (Edmund Optics) positioned at the end of a light guide and a collimating lens. The light intensity was measured using an International Light radiometer. After polymerization, unreacted monomer was removed with three five minute water rinses, followed by air drying. To obtain sufficient replicates in the microarray experiments, each condition investigated was polymerized on at least 2-3 arrays in 2-3 separate polymerization sessions.

Film thickness measurements. Polymer film thicknesses were measured using a Dektak 6M surface profilometer with 12.5 μm diameter tip and a stylus force of 1 mg.

Fluorescent imaging of films. Cells stained by fluorescent polymerization-based signal amplification were imaged by confocal scanning laser microscopy (Zeiss LSM 510 instrument) with a 40× oil objective. Yellow/green NP fluorescence on the microarray surfaces was measured using a Leica MZ FLIII stereomicroscope (Leica Microsystems, Wetzlar, Germany) with the blue filter set. Exposure times in the range of 5 seconds to 60 seconds were used, depending on the intensity of the fluorescent signal. A Cy3 Scanner Calibration slide was imaged at each exposure time to identify appropriate scaling factors for the different exposure times, such that all data could be plotted using the same arbitrary fluorescence scale. Crimson and dark red NP fluorescence on the microarray surfaces were measured using the red channel of an Agilent Technologies Microarray Scanner. Significant positive signals are those that have a signal to noise (S/N) greater than 3, where [S/N=(signal−background signal)/(standard deviation of the background signal)]. The film fluorescence values have been normalized to account for the differences in relative quantum yield between NPs of different sizes or different surface chemistries, utilizing the relative quantum yields provided in Table 1. Additionally, for NPs of the same color, but different sizes or surface-functionalities, the film fluorescence values were normalized to account for the slight differences in the NP fluorophore content per mass, as determined by absorbance measurements (Table 1). In this way, the reported fluorescence intensities are more indicative of the mass of NPs that has been immobilized in the films, rather than simply variations in the manufacturing of these NPs.

Statistical Analysis. In cases where a “gain value” is reported, linear regression with a least squares parameter estimation was performed to best fit a line of the form y=mx (intercept set to 0) to the data. The slope “m” is termed “gain” and the 95% confidence interval on the slope is indicated.

Mesh size determinations. Mesh sizes were estimated by measuring the equilibrium swelling ratio (Q) of the gels as described by Bryant and Anseth (2006, Scaffolding in Tissue Engineering. Taylor and Francis Group, Boca Raton, pp 71-90). Photoinitiation of the indicated monomer formulations was achieved with 4 μM eosin-isothiocyanate to generate disc shaped hydrogels of approximately 1 mm thick and 8 mm in diameter. The gels were rinsed 24 hours with several changes of water. The swollen weight (Mₛ) was recorded and then the gels were dried under vacuum at 37° C. for three days after which the dried weight (M_{d}) was recorded. Q was calculated as shown in equation 2-1, where ρₛ is the solvent density (1 mg/mL) and ρₚ is the polymer density, 1.12 mg/mL for PEG gels and 1.35 mg/mL for polyacrylamide gels.

$$Q = 1 + \frac{{ρ}_{p}}{{ρ}_{s}} ( \frac{{M}_{s}}{{M}_{d}} - 1 ) \qquad ( 2 \text{-} 1 )$$

The average molecular weight between crosslinks (M_{c}) is calculated as shown in equation 2-2, where V₁ is the molar volume of water (18 mL/mol), v_{2,s} is the equilibrium polymer volume fraction (1/Q), X₁₂ is the solvent-polymer interaction parameter, 0.43 for PEG-based gels and 0.48 for polyacrylamide gels. Except for the gels comprised of PEGDA with no PEGA, the term 2/Mₙ is neglected as M_{c}<<Mₙ.

$$\frac{1}{{\overline{M}}_{c}} = - \frac{( 1 / {ρ}_{p} {V}_{1} ) [ ln ( 1 - {v}_{2 , s} ) + {v}_{2 , s} + {X}_{12} {v}_{2 , s}^{2} ]}{[ {v}_{2 , s}^{1 / 3} - \frac{{v}_{2 , s}}{2} ]} - \frac{2}{{\overline{M}}_{n}} \qquad ( 2 \text{-} 2 )$$

Finally, the average mesh sizes (ε) for the swollen hydrogels are calculated as shown in equation 2-3, where Cₙ is the characteristic ratio of the polymer (4 for PEG-based gels and 14.8 for the polyacrylamide gels), l is the bond length, and n is the number of bonds between the crosslinks.

εν_{2,s}^{-1/3}Cₙ^{1/2}n^{1/2}l (2-3)

The observation that PEGA with no added crosslinker readily forms a crosslinked gel highlights the fact that a significant amount of chain transfer occurs in the PEGA and PEGDA systems, which creates ambiguity concerning the chemical composition of the chains between crosslinks. Thus, the average mesh sizes for the PEG-based gels were calculated by two distinct methods to provide reasonable bounds on the probable average mesh sizes: 1) n=2M_{c}/Mᵣ, where Mᵣ is the molecular weight of the repeating monomer unit assuming no chain transfer; and, 2) assuming extensive chain transfer, n=3M_{c}/Mᵣ, where Mᵣ is the molecular weight of the PEG repeat unit. The value of three in the second method arises from the fact that the PEG repeat unit (COO) contains three bonds. In the case of polyacrylamide, n=2M_{c}/Mᵣ, where Mᵣ is 71 g/mol.

### Results and Discussion

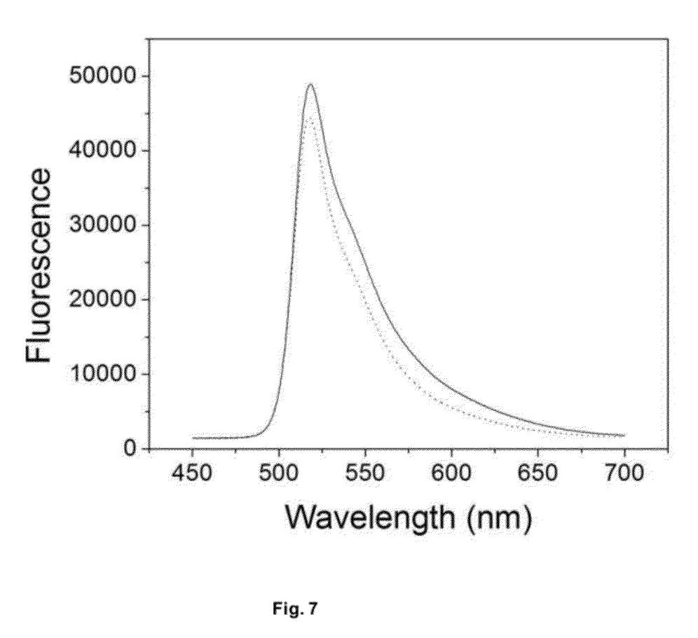

Compare the photoluminescence of NPs in solution and in the polymer film. The photoluminescent properties of these NPs are not expected to change upon encapsulation in polymer films, as the fluorophores in the NPs are already embedded in the NP polymer matrix. To verify that encapsulation in polymer does not dramatically alter NP photoluminescence, emission spectra were compared between free 20 nm yellow/green NPs in water (0.05 wt % NPs) and NPs that had been entrapped in a 1 cm thick polyacrylamide film (FIG. 7). Entrapment in polyacrylamide resulted in only a 9% drop in the maximum fluorescent signal and the shape of the emission spectra was not significantly altered. These results indicate that NP photoluminescence is not altered in a meaningful manner either by the polymerization reaction or by being embedded in additional polymer matrix.

Effect of NP size. To ascertain the impact of NP diameter on the ability of fluorescent NPs to become encapsulated in films generated from surface-mediated photoinitiation, polymerizations were performed with monomer solutions containing fluorescent NPs of a variety of distinct diameters ranging from 20 nm to 200 nm. Additionally, a microarray format was employed such that a wide range of eosin photoinitiator surface densities were evaluated simultaneously, enabling investigation of the effect of NP size at various initiator surface densities. Using various eosin initiator surface densities ensures that the observed trends for NP size are generalizable to varying initiation rates. Finally, two different monomer formulations were employed, an acrylamide-based formulation and a PEGDA-based formulation.

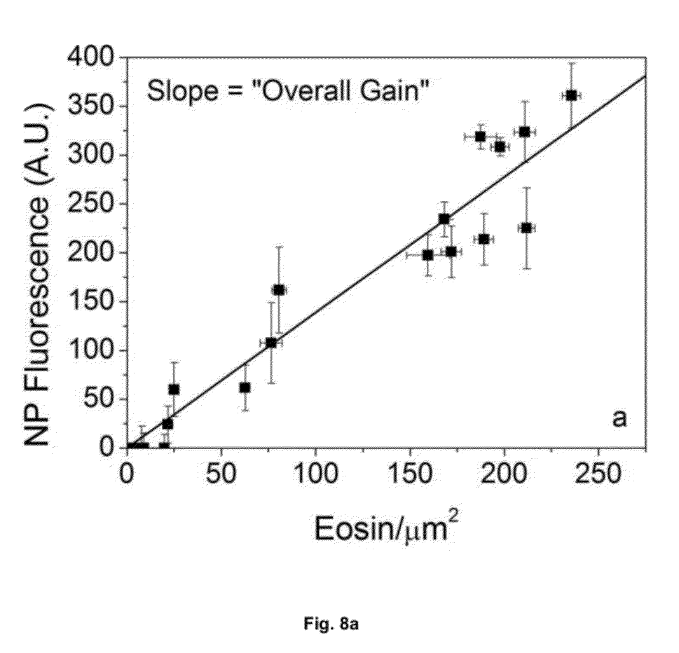

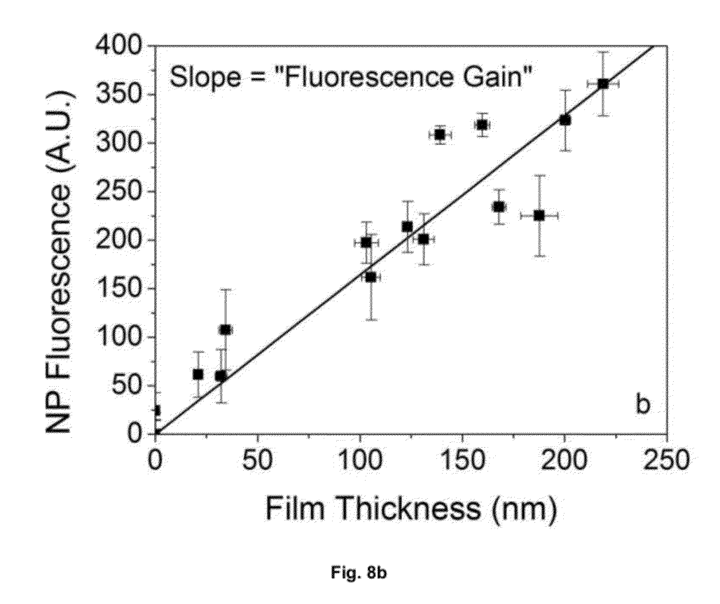

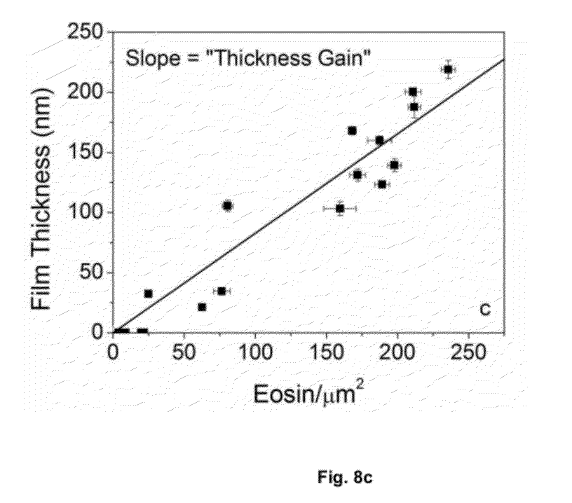

In the context of generating fluorescent films via NP entrapment, one can envision achieving improved fluorescent signals in two ways: firstly, by generating thicker films; or, secondly, by incorporating a higher density of NPs into the films. To take into account both these contributions, the data presented here are evaluated not only for overall fluorescence generated as a function of eosin initiator surface density, but also for both how thick the films are and how much fluorescence is seen as a function of film thickness. FIG. 8 shows a typical data set comprising the results from photopolymerizations of an acrylamide monomer formulation containing 0.05 wt % 100 nm yellow/green NPs. Three terms are introduced to facilitate analysis of these data: 1) “overall gain” refers to the slope on the plot of eosin surface density (x) vs. film fluorescence (y) (FIG. 8a) and is indicative of the increase in film fluorescence observed as photoinitiator surface density increases; 2) “fluorescence gain” denotes the slope on the plot of film thickness (x) vs. film fluorescence (y) (FIG. 8b) and signifies the amount of increase in film fluorescence observed with an increase in film thickness; and, 3) “thickness gain” refers to the slope on the plot of eosin surface density (x) vs. film thickness (y) (FIG. 8c) and describes the increase in film thickness associated with a particular increase inphotinitiator surface density. In all cases, the gain is reported to be the least squares fit of the proportionality constant for the two relevant factors. The three types of gains are related in that the fluorescence gain multiplied by the thickness gain is roughly equal to the overall gain; however, due to the error associated with linear regression, this relationship is not exact. Nearly all the data sets were sufficiently linear such that the p values for the slopes were many orders of mangitude less than one. The exceptions were the overall gain and the fluorescence gain for the 200 nm dark red NPs depicted in FIG. 10, whose p values were greater than 0.05. The fact that the film fluorescence typically increases monotonically with film thickness suggests that the NPs are evenly distributed throughout the thickness of the films, which is consistant with previously published results that also reported a linear relationship between film thickness and film fluorescence (Avens and Bowman 2010, Acta Biomater 6:83-89). A comparison of the gain values for polymerizations performed under different conditions enables determination of which conditions yield improved fluorescence amplification, and whether that amplification is a result of increased film thickness, a higher density of NP incorporation, or both.

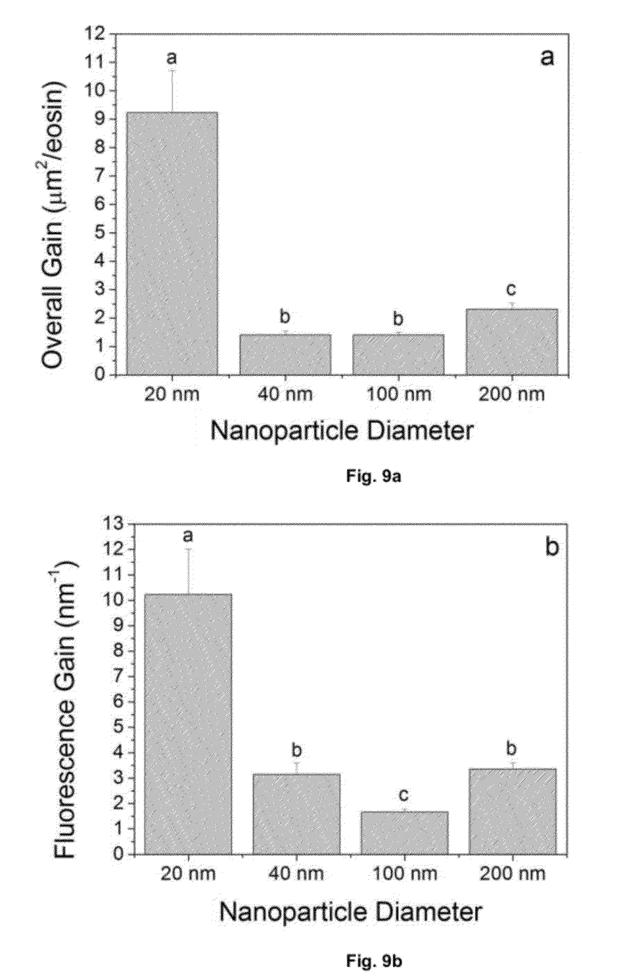

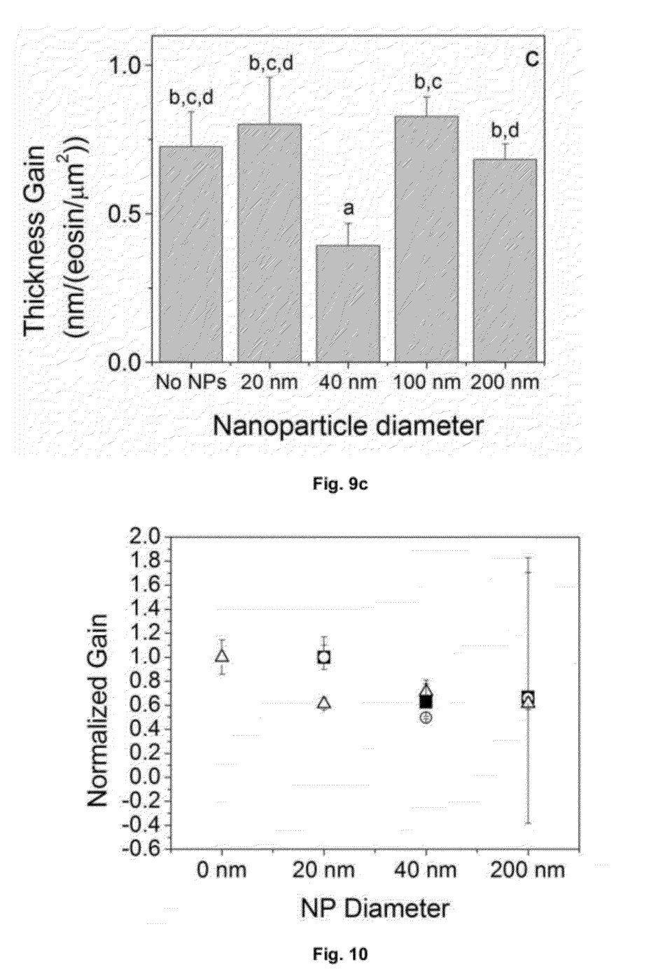

Surface-mediated initiation of polymerization of an acrylamide monomer formulation with varying sizes of yellow/green NPs (0.05 wt %) reveals that the smallest NP diameter, 20 nm, results in four-fold to six-fold greater overall gain compared to the larger NP sizes. These results indicate that the 20 nm NP size favors immobilization of a higher mass of NPs onto the surface (FIG. 9a). Further, as the fluorescence gains are three-fold to six-fold higher for the 20 nm NPs (FIG. 9b), it is concluded that polymerization with 20 nm NPs enables a higher density of NPs (on a mass-basis) to become entrapped in the films. The ability of 20 nm NPs to incorporate better into the films is the dominant contribution to the overall gain, as there is no clear trend between thickness gain and NP diameter (FIG. 9c). Indeed, FIG. 9c demonstrates that film thickness is determined by the eosin photoinitator surface density and shows no clear correlation with NP size.

Similar experiments were also performed using a PEGDA monomer formulation with varying sizes of dark red NPs. To facilitate a more concise presentation of the data, the gains were normalized to a scale of 0-1 by dividing each gain by the largest gain in the data set. In this way, it is possible to plot the overall gain, fluorescence gain, and thickness gain all on a single chart, as shown in FIG. 10. Tables 5-8 are tables of all the non-normalized gain values for the figures in this example that display normalized gains. In agreement with the acrylamide results, it is found that the nm NP size favors improved fluorescence signal compared to the 40 nm NP size, primarily as a result of better incorporation of the 20 nm NPs into the films, rather than by growth of thicker films (FIG. 10). The increase in overall gain and fluorescence gain associated with the 20 nm particles in PEGDA is not as dramatic as was observed with the acrylamide formulation. This behavior suggests that factors other than NP size are more important in determining NP incorporation in the PEGDA system than in the acrylamide system. Additionally, the large error associated with the 200 nm NPs is attributed to sporatic non-specific binding of the 200 nm particles to the surface, and indicates that this size NP is unsuitable for use with the PEGDA monomer formulation in biodetection applications that require quantiation and specificity.

The enhanced incorporation of smaller NPs likely arises from the interfacial nature of the surface-initiated polymerization and the evolving crosslinked network that is forming. Though the polymerization is initiated from a uniform surface, heterogeneities and differential extension of the polymer film into the bulk monomer will undoubtedly arise, particularly in these crosslinked materials that are notorious for microgel formation and heterogeneity (Hutchison and Anseth 2001, Macromol Theory Simul 10:600-607; Kloosterboer 1988, Adv Polym Sci 84:1-61). The smaller the NP is, the more likely it is to become entrapped in this evolving structure.

Effect of NP surface Chemistry. In light of the evolving interface between the polymerizing mixture and the bulk monomer, it is anticipated that chemical interactions between the polymer and functional groups on the NP surface are likely to impact incorporation of fluorescent NPs into polymer films. To investigate the effect of surface modifications, a comparison was made between carboxylate vs. amine modification of the fluorescent NPs. FIG. 11 shows that amine-functionalized NPs are preferentially incorporated into the polyacrylamide films compared to the carboxylate-functionalized NPs, as evidenced by their higher fluorescence gain; however, the amine-functionalized NPs yield thinner films than the carboxylate-functionalized NPs. The net effect is that the amine-functionalized NPs trend towards having higher overall gains than the carboxylate NPs, though this difference is not found to be significant (α<0.05). The polymerization is performed at pH=9, much higher than the pK, of carboxylic acid (˜5) and lower than the pKₐ of ammonium ions (˜11) (Jones 1997, Organic Chemistry. WW Norton & Company, New York, pp 238 & 1087); therefore, during polymerization, the amine-functionalized particles have positive surface charges, while the carboxy-functionalized particles have negative surface charges. It is not immediately apparent that either of these functional groups would react preferentially with polyacrylamide, as the carboxyl group on acrylamide has a partial negative charge, while the amine on acrylamide has a partial positive charge. However, amines are known to undergo chain transfer more readily than carboxylates, even being capable coinitiators for a variety of photosensitization systems, including eosin. Generation of radicals on the amine-functionalized NPs would likely lead to covalent bond formation with the polymer network through polymerization and subsequent crosslinking from the radical formed as a result of the chain transfer process. This hypothesis also explains the thinner films associated with the amine-functionalized particles as the additional chain transfer will slow the polymerization.

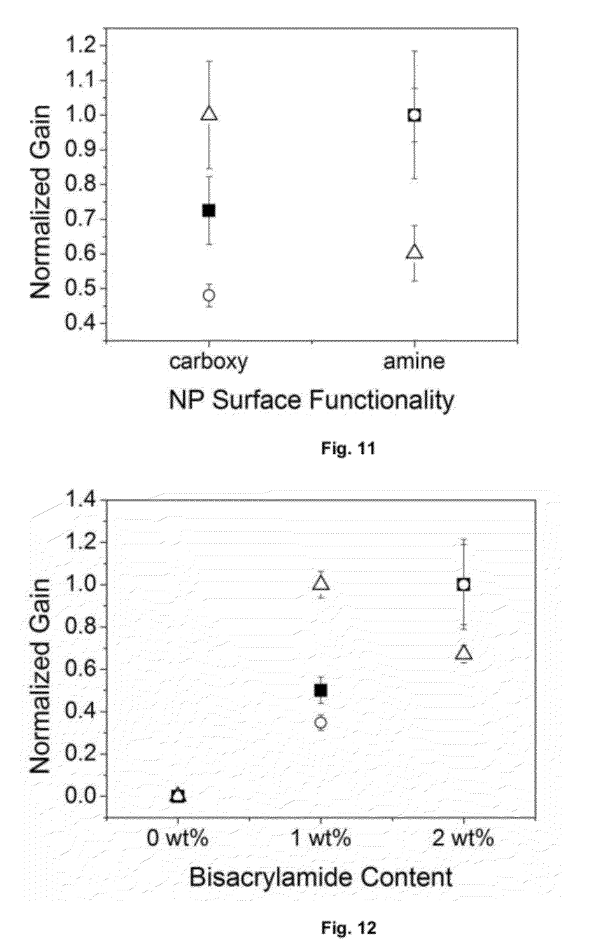

Effect of Crosslinker Concentration. Increasing the crosslinking density of a polymer network increases the polymer modulus and decreases the average mesh size. If the average mesh size is on the order of the particle diameter, then changes in the crosslinking content of the monomer formulation would be expected to impact the ability of these gels to entrap fluorescent NPs effectively. To investigate how crosslinking density affects the incorporation of nanoparticles during a surface-mediated polymerization, polymerizations were performed with varying bisacrylamide crosslinker concentrations (0, 1, and 2 wt %) added to an acrylamide monomer formulation. It was found that the higher crosslinking agent content favors incorporation of NPs into the polymer films, yielding a more than 2-fold increase in fluorescence gain, and ultimately resulting in a greater number of fluorescent species immobilized on the surface (i.e. greater overall gain) (FIG. 12). When bisacrylamide is absent, no polymer film is detected by profilometry or by fluorescence. In the absence of any bisacrylamide crosslinking agent, the polymerization rate is too slow to generate detectable films under these conditions.

A similar trend is seen in the gels formed from varying ratios of PEGDA and PEGA that achieve significant variations in crosslinking density and network mesh size. As the PEGDA content is increased, the fluorescence yield and overall yield also increase (Table 2), indicating more efficient trapping of the fluorescent NPs. On the other hand, the effect of crosslinker content on thickness gain does not exhibit any clear trend. This behavior is likely due to the fact that a more crosslinked gel has two conflicting influences: 1) it leads to an increased polymerization rate as termination becomes less facile; and, 2) in a surface-mediated eosin polymerization, the more crosslinked gel limits diffusion of MDEA to the surface and hinders its surface initiated radicals, when formed, from reaching the monomer-rich regions, thereby slowing the rate. Also, unlike the polyacrylamide system, PEGA polymerized without any additional crosslinking agent does form a detectable, insoluble film, most likely due to a small amount of diacrylate impurity present in the monoacrylate and/or chain transfer reactions which lead to crosslink formation in combination with termination by combination.

To investigate further the effect of crosslinking density on the incorporation of fluorescent NPs, the molecular weight between crosslinks (M_{c}) and the average mesh size were determined for hydrogels created using each monomer formulation described in FIG. 12 and Table 2. In the polyacrylamide system, 2 wt % bisacrylamide resulted in approximately 60% smaller average mesh sizes (9 vs. 14 nm) than the gels made with 1 wt % bisacrylamide (Table 3). For the PEG gels, variations in the crosslinker content yielded a range of mesh sizes from 2-19 nm (Table 4), demonstrating the possibility for up to a tenfold variation in mesh size in gels generated from these monomer formulations. The diameters of the NPs employed in the crosslinker experiment (24+/−4 nm for both the yellow/green and the Crimson NPs) are slightly greater than the largest average mesh size determined here, indicating that it is unlikely these NPs diffuse out of the gel once they are entrapped; however, at the monomer-polymer interface where NP incorporation occurs, the NPs are interacting with nascent polymer networks with incomplete mesh structures, which are likely larger than the final mesh size within the bulk of the gel. It seems probable that average internal mesh sizes in the range of 2-19 nm would correspond to incomplete mesh structures at the polymer-monomer interface that are on a size scale that would impact the efficiency of trapping 24 nm diameter NPs. It is posited that gels with smaller internal mesh size have incomplete mesh structures at the polymer-monomer interface that are smaller and better able to trap the NPs. Secondly, the gels with smaller mesh sizes necessarily have a faster rate of mesh formation for a given polymerization rate, providing less time for NPs at the polymerization front to diffuse away before becoming entrapped. Thus, the enhanced NP entrapment observed with increased crosslinker content is likely attributable to a combination of changes in the average mesh size at the polymer-monomer interface and differences in the rate of mesh formation.

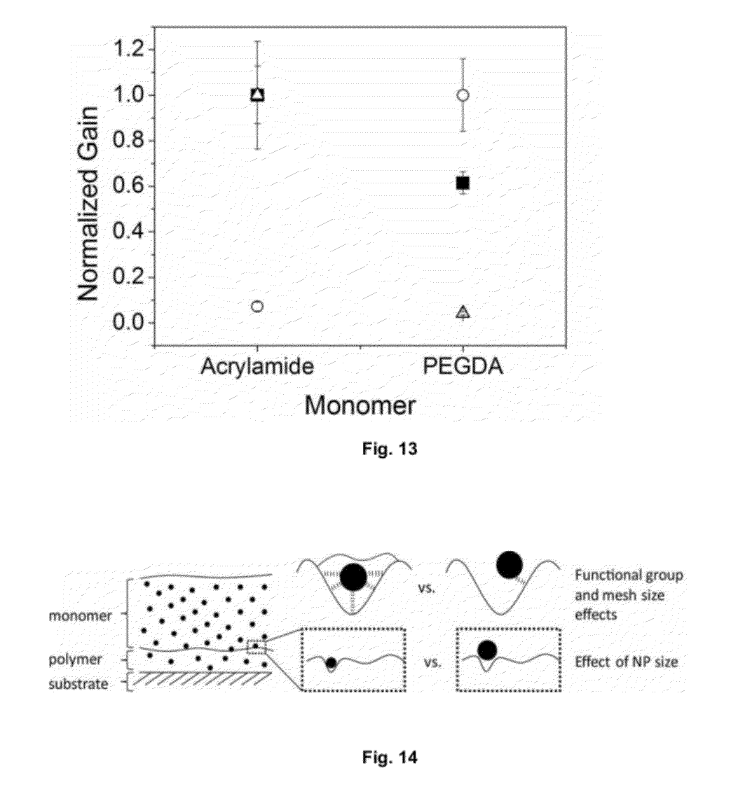

Effect of Monomer type. Monomer selection has been shown to have a significant effect on the thickness of polymer films generated from surface-mediated eosin initiation of polymerization (Avens et al. 2008, Polymer 49:4762-4768). In addition to yielding films of differing thicknesses, monomer choice is also expected to impact how well NPs incorporate into the growing films, based on chemical interactions between the polymer and the NP surface, as well as the mechanical properties of these films. To determine how the two monomer formulations employed here affect the immobilization of fluorescent nanoparticles, surface-mediated polymerizations were carried out with both an acrylamide and PEGDA monomer formulation in the presence of 0.05 wt % 20 nm Dark red NPs. Not unexpectedly, the acrylamide formulation has a more than 20-fold greater thickness gain as compared to the PEGDA system (FIG. 13), which is consistent with previous reports on similar monomer systems (Avens et al. 2008, Polymer 49:4762-4768). The overall gain is also larger for the acrylamide system though by less than two fold. Strikingly, the fluorescence gain of the PEGDA system is 10-fold higher than the acrylamide formulation, highlighting that the PEGDA formulation is much more efficient at trapping fluorescent NPs though its polymer films are significantly thinner. This difference is likely attributable to the smaller average mesh size of the PEGDA formulation (2-5 nm) as compared to the acrylamide formulation (9 nm) (Tables 2 and 3), leading to more facile NP entrapment. Secondly, the NPs are comprised of a polystyrene core, surrounded by a PEG shell which is functionalized with carboxylate groups. Thus, it is reasonable to expect that the NPs are more compatible chemically with the PEGA/PEGDA environment, interacting more favorably with PEG chains in these polymers than with the relatively more hydrophobic polyacrylamide. A summary of the posited important factors that affect NP incorporation into films generated by surface-mediated initiation of polymerization are shown in FIG. 14. Smaller particle sizes are seen to incorporate more efficiently into the films, likely due to their ability to become entrapped in the heterogeneous monomer-polymer interface. Additionally, surface functionality of the NPs and monomer choice also affect the entrapment of fluorescent NPs through non-covalent and possibly also covalent interactions. Finally, it is hypothesized that the favorable influences of smaller mesh sizes and faster rates of mesh formation at the monomer-polymer interface contribute to the improved NP incorporation seen with higher crosslinker content. Knowledge of these factors enables selection of NP and monomer attributes that are most suitable for specific applications that rely on surface-mediated initiation of polymerization to immobilize fluorescent NPs.

Impact on Sensitivity in a Biodetection Application. Fluorescent polymerization-based amplification (FPBA) has been demonstrated previously to be a sensitive array-based biodetection platform for signal amplification that enables a100-fold improvement in sensitivity as compared to traditional fluorescence detection methods (Avens and Bowman 2010, Acta Biomater 6:83-89; Hansen et al. 2008, Anal Bioanal Chem 392:167-175). FPBA relies on surface-mediated initiation of polymerization to entrap fluorescent NPs within PEGDA or polyacrylamide polymer films. Specifically, the assay employs DNA or protein probes that are coupled to eosin photoinitiators, thereby immobilizing eosin photoinitiators on the surface wherever the probe has bound its target. In the last step of the assay, the surface is contacted with monomer, MDEA coinitiator, and fluorescent NPs and exposed to visible light, generating a highly fluorescent film specifically in regions of the surface where target is present. To achieve the highest sensitivity for fluorescence detection, the mass of fluorescent NPs immobilized per surface area in response to the eosin-initiated polymerization is maximized.

The FPBA array-based biodetection platform was used to assess the combined effect of selecting monomer and NP attributes that are more or less favorable for immobilization of fluorescent NPs to the surface. As a model system for biodetection, microarray slides were used that contain a range of biotin-labeled antibody surface densities. These slides were contacted with eosin-labeled streptavidin which specifically binds to biotin, thereby immobilizing eosin photoinitiators to the surface. The biotin-streptavidin system is a commonly used label-probe combination employed in many detection assays. Two different monomer formulations were compared: 1) an acrylamide monomer formulation containing 2 wt % crosslinker and 20 nm yellow/green NPs; and, 2) a formulation containing 1 wt % PEGDA, 21 wt % PEGA and 100 nm yellow/green NPs. Based on the aforementioned NP-incorporation studies, the first formulation is expected to generate significantly stronger amplified fluorescence signals and allow more sensitive detection due to the smaller NP diameter, the higher crosslinker content, and the ability of acrylamide to grow thicker films.

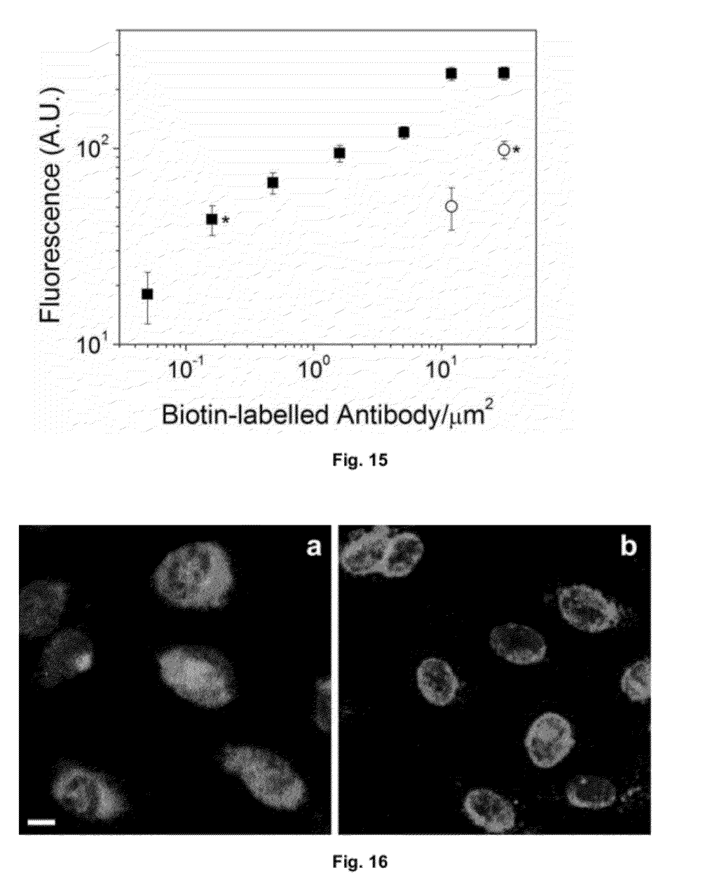

Indeed, the acrylamide formulation with 20 nm NPs is found to enable detection of as few as 0.16 biotin-labeled antibodies per micron², or 40 zeptomole surface-bound target molecules, while the PEGA/PEGDA formulation with 100 nm NPs is approximately 200-fold less sensitive with a detection limit of 31 biotin-labeled antibodies per square micron (FIG. 15). A direct comparison of the magnitude of fluorescence intensity generated by the two formulations is not readily obtained from these data, as the signal generated from the acrylamide formulation has started to saturate at the antibody surface densities that are required to see significant signal from the PEGDA/PEGA system. Additionally, it should be noted that the threshold for significant signal (S/N>3) was higher in the PEGA/PEGDA system because the non-specific polymerization background on these surfaces was higher and more variable. These results demonstrate that selection of the appropriate monomer and NP combination is important for designing a biodetection platform that relies on entrapment of NPs by surface-mediated initiation of polymer film growth.

Impact on the ability to obtain localized staining of a small cellular feature. Recently, FPBA has been extended to fluorescent immunocytochemistry, enabling enhanced fluorescence and more sensitive detection compared to traditional fluorescent immunocytochemistry methods which employ direct fluorescent labeling of the antigenic or protein probe (Avens H J et al., J Histochem Cytochem, vol. 59 no. 1, 76-87. In this format, cells fixed onto glass slides are probed with antibodies that specifically bind a target of interest within the cell. Eosin photoinitiators are either coupled to the primary or secondary antibody, or alternatively, eosin-labeled streptavidin is used in conjunction with biotin-labeled antibodies. In the last step of the assay, the cells are contacted with a monomer formulation containing fluorescent NPs and are exposed to light, yielding the formation of highly fluorescent films specifically on structures in the cell where the antigenic probe has bound its target protein. Unlike the microarray platform in which generation of thick films is acceptable, for a cell imaging application, the generation of excessively thick films is undesirable because it reduces the spatial resolution of the staining process. Therefore, it is expected that a PEGDA formulation would be preferable to an acrylamide formulation since the former is capable of immobilizing a higher density of fluorescent NPs into the polymer film. This hypothesis was tested by performing FPBA with each formulation to detect nuclear pore complex proteins (NPC) which are localized within the nuclear envelope. As shown in FIG. 16, the acrylamide formulation yields an unacceptably diffuse ring of staining around the nucleus that reaches approximately 10 microns thick in some regions. Conversely, staining with PEGDA results in a more spatially confined stain that appears to be on the order of 1-2 microns thick, specifically localized around the targeted features at the edge of the nucleus.

These two biodetection examples demonstrate the importance of understanding the NP incorporation behavior and their impact on polymerization as each different application places distinct requirements on the polymerization and detection process. Here, the optimal NP-monomer combination is dramatically different depending on whether one requires maximum sensitivity or whether one desires maximum spatial resolution

### Conclusions

The results presented here highlight the fact that careful consideration of monomer and NP selection is essential to achieving the desired properties in applications that employ surface-mediated polymerizations to entrap NPs. In choosing a monomer formulation, higher crosslinker content tends to be more efficient at entrapping NPs, while consideration of the interactions between the polymer and the NP surface is also important. Choice of suitable NP attributes requires consideration of both NP size and surface chemistry, with smaller sizes tending to favor entrapment in polymer matrices. Although this study employed relatively low NP concentrations (0.05 wt %) such that light attenuation of the NPs would not excessively hinder photoinitiation, it is expected that the trends outlined here will be similar and perhaps even more pronounced with increased NP loading. It is additionally anticipated that these findings will be helpful in designing approaches for surface-mediated generation of polymeric nanocomposites comprised of other polymers, polymerization types, and NP types. See also Avens et al. (2011), Journal of Nanoparticle Research 13 (1) 331-346, hereby incorporated by reference in its entirety.

TABLE 1
Characteristics of NPs used for these studies.^{a}

| | | Nominal | Actual | Rela- | |
|---|---|---|---|---|---|
| | Surface | diameter | diameter | tive | Relative |
| Color | functionality | (nm) | (nm) | QY^{b} | Absorbance^{c} |
| Yellow/ | Carboxylate | 20 | 24 +/− 4 | 0.33 | 1.00 |
| green | | | | | |
| Yellow/ | Carboxylate | 40 | 43 +/− 6 | 0.47 | 1.37 |
| green | | | | | |
| Yellow/ | Carboxylate | 100 | 100 +/− 6 | 0.39 | 0.85 |
| green | | | | | |
| Yellow/ | Carboxylate | 200 | 210 +/− 10 | 0.17 | 0.93 |
| green | | | | | |
| Yellow/ | Amine | 200 | 190 +/− 11 | 0.18 | 0.93 |
| green | | | | | |
| Dark red | Carboxylate | 20 | 24 +/− 3 | 1.60 | 1.00 |
| Dark red | Carboxylate | 40 | 36 +/− 5 | 1.45 | 0.46 |
| Dark red | Carboxylate | 200 | 210 +/− 10 | 0.46 | 0.46 |
| Crimson | Carboxylate | 20 | 24 +/− 4 | 1.00 | N/A |

^{a}These data are provided by Invitrogen on the certificate of analysis for each NP Fluosphere product.
^{b}For yellow/green NPs, the QY is relative to a solution in methanol of the dye used to prepare the NPs. For dark red NPs and the crimson NPs, the QY is relative to a solution in chloroform of the dye used to prepare the NPs.
^{c}For NPs of the same color, but different sizes or surface functionalities, relative absorbance values were determined. The absorbance values are per mass of NPs, and the 20 nm NP absorbance values are set to 1.0.

TABLE 2
Gain values mesaured for fluorescent films formed from varying
concentrations of PEGA and PEGDA.^{a}

| | Overall Gain | Fluorescence | |
|---|---|---|---|
| Diacrylate/ | (NP | Gain | |
| Monoacrylate | Fluorescence/ | (NP | Thickness Gain |
| (wt %/wt %) | (eosin/μm²)) | Fluorescence/nm) | (nm/(eosin/μm²) |
| 0/22 | 26 (+/−3)^{A} | 220 (+/−30)^{A} | 0.094 (+/−0.023)^{A,B} |
| 1/21 | 27 (+/−4)^{A} | 390 (+/−30)^{B} | 0.066 (+/−0.013)^{B} |
| 11/11 | 55 (+/−6)^{B} | 370 (+/−60)^{B} | 0.13 (+/−0.02)^{A} |
| 22/0 | 57 (+/−5)^{B} | 570 (+/−110)^{C} | 0.084 (+/−0.014)^{B} |

^{a}Varying PEGDA and PEGA concentrations are added to 210 mM MDEA, 35 mM VP, and 0.05 wt % 20 nm Crimson NPs in water. (22 wt % PEGDA is 420 mM, and 22 wt % PEGA is 650 mM.) Polymerizations were initiated by a 30 minute exposure to 40 mW/cm² light of wavelengths greater than 480 nm. Capital letters denote groups of conditions that are statistically different (α = 0.05).

TABLE 3
Estimated molecular weight between crosslinks (M_{c}) and
mesh size in polyacrylamide gels prepared with varying
amounts of bisacrylamide crosslinker.^{a}

| Bisacrylamide Content | | |
|---|---|---|
| (wt %) | M_{c} (g/mol) | Mesh size (nm) |
| 1 | 5.5 (+/−0.1) × | 14 (+/−0) |
| | 10³ | |
| 2 | 2.9 (+/−0.1) × | 9.1 (+/−0.2) |
| | 10³ | |

^{a}Gels prepared with 5.2M acrylamide, 210 mM MDEA, 35 mM VP, and 4 μM eosin isothiocyanate in water. 2 wt % bisacrylamide is 130 mM. Polymerizations were initiated by a 30 minute exposure to 40 mW/cm² light of wavelengths greater than 480 nm.

TABLE 4
Estimated molecular weight between crosslinks (M_{c}) and mesh size
in PEG gels prepared with varying amounts of PEGDA crosslinker.^{a}

| Diacrylate/Monoacrylate | | Mesh size range |
|---|---|---|
| (wt %/wt %) | M_{c} (g/mol) | (nm)^{b} |
| 0/22 | 1.2 (+/−0.1) × 10⁴ | 5.6-19 |
| 1/21 | 7.9 (+/−0.7) × 10³ | 4.4-14 |
| 11/11 | 2.2 (+/−0.1) × 10³ | 2.0-6.5 |
| 22/0 | 2.4 × 10²-1.5 × 10^{3c} | 2.0-5.0 |

^{a}Gels prepared with 210 mM MDEA, 35 mM VP, and 4 mM eosin-isothiocyanate in water. 22 wt % PEGDA is 420 mM, and 22 wt % PEGA is 650 mM. Polymerizations were initiated by a 30 minute exposure to 40 mW/cm² light of wavelengths greater than 480 nm.
^{b}The low end of the range is obtained by assuming that the chain between crosslinks is mainly comprised of carbon-carbon linkages. The high end of the range is obtained by assuming that the chain between crosslinks mostly consists of PEG-backbone.
^{c}M_{c} is calculated with and without taking into account chain ends.

TABLE 5
Gain values mesaured for PEGDA films that encapsulate
NPs of different sizes.^{a}

| NP | Overall Gain | Fluorescence Gain | |
|---|---|---|---|
| Diameter | (NP fluorescence/ | (NP Fluorescence/ | Thickness Gain |
| (nm) | (eosin/μm²)) | nm) | (nm/(eosin/μm²) |
| No NPs | — | — | 0.19 (+/−0.03)^{A} |
| 20 | 7.5 (+/−1)^{A} | 62.5 (+/−6)^{A} | 0.12 (+/−0.01)^{B} |
| 40 | 4.7 (+/−1)^{B} | 31.0 (+/−1)^{B} | 0.13 (+/−0.01)^{B} |
| 200 | 5.0 (+/−9)^{A,B} | 41.0 (+/−65)^{A,B} | 0.12 (+/−0.01)^{B} |

^{a}Polymer films are generated by surface mediated initiation with eosin using a PEGDA monomer formulation (420 mM PEGDA, 210 mM MDEA, and 35 mM VP in water) containing carboxylate-functionalized dark red NPs of various sizes (0.05 wt %). Polymerizations were initiated by a 30 minute exosure to 40 mW/cm² light of greater than 480 nm. Fluorescence measurements were obtained using the red channel of an Agilent microarray scanner. Films generated in the absence of NPs were not fluorescent. Capital letters denote groups of conditions that are statistically different (α = 0.05).

TABLE 6
Gain values mesaured for polyacrylamide films that encapsulate
NPs of different surface functionalities.^{a}

| | Overall Gain | Fluorescence | |
|---|---|---|---|
| Surface | (NP Fluorescence/ | Gain (NP | Thickness Gain |
| Functionality | (eosin/μm²)) | Fluorescence/nm) | nm/((eosin/μm²) |
| Carboxylate | 3.1 (+/−0.4)^{A} | 3.5 (+/−0.2)^{A} | 0.92 (+/−0.14)^{A} |
| Amine | 4.2 (+/−0.8)^{A} | 7.2 (+/−0.6)^{B} | 0.56 (+/−0.07)^{B} |

^{a}Polymer films are generated by surface mediated initiation with eosin using an acrylamide monomer formulation (5.2M acrylmaide, 130 mM bisacrylamide, 210 mM MDEA and 35 mM VP in water) with 0.05 wt % 200 nm yellow/green nanoparticles. Polymerizations were initiated by a 30 minute exosure to 40 mW/cm² light of greater than 480 nm. Fluorescence measurements were obtained using a Leica stereomicroscope with the blue filter. Capital letters denote groups of conditions that are statistically different (α = 0.05).

TABLE 7
Gain values mesaured for fluorescent polyacrylamide films
formed from varying concentrations of bisacrylamide crosslinker.^{a}

| | Overall Gain | | |
|---|---|---|---|
| Bisacrylamide | (NP | Fluorescence Gain | |
| Content | Fluorescence/ | (NP Fluorescence/ | Thickness Gain |
| (wt %) | (eosin/μm²)) | nm) | (nm/(eosin/μm²) |
| 1 | 4.2 (+/−0.6)^{A} | 2.4 (+/−0.2)^{A} | 1.8 (+/−0.1)^{A} |
| 2 | 8.8 (+/−1.5)^{B} | 7.0 (+/−1.5)^{B} | 1.2 (+/− 0.08)^{B} |

^{a}Different concentrations of bisacrylamide are added to 5.2M acrylmaide, 210 mM MDEA, 35 mM VP, and 0.05 wt % 20 nm carboxylate-functionalized yellow/green NPs in water. (2 wt % bisacrylamide is 130 mM.) Polymerizations were initiated by a 30 minute exosure to 40 mW/cm² light of greater than 480 nm. Fluorescence measurements were obtained using a Leica stereomicroscope with the blue filter. Capital letters denote groups of conditions that are statistically different (α = 0.05).

TABLE 8
Gain values mesaured for fluorescent films formed from
different monomer types.^{a}

| | Overall Gain | Fluorescence Gain | |
|---|---|---|---|
| | (NP fluorescence/ | (NP Fluorescence/ | Thickness Gain |
| Monomer | (eosin/μm²)) | nm) | (nm/(eosin/μm²) |
| Acrylamide | 34 (+/−8)^{A} | 13 (+/−6)^{A} | 2.5 (+/−0.3)^{A} |
| PEGDA | 21 (+/−2)^{B} | 175 (+/−31)^{B} | 0.10 (+/−0.02)^{B} |

^{a}Polymer films are generated by surface mediated initiation with eosin using either an acrylamide monomer formulation (5.2M acrylamide with 130 mM bisacrylamide) or a PEGDA formulation (420 mM PEGDA), each with 210 mM MDEA, 35 mM VP, and 0.05 wt % 20 nm carboxylate-functionalized dark red NPs. Polymerizations were initiated by a 30 minute exosure to 40 mW/cm² light of greater than 480 nm. Fluorescence measurments were obtained using the red channel of an Agilent microarray scanner. Capital letters denote groups of conditions that are statistically different (α = 0.05).

### REFERENCES

Allegra G, Raos G, Vacatello M (2008) Theories and simulations of polymer-based nanocomposites: From chain statistics to reinforcement. Prog Polym Sci 33:683-731.
An Y, Hubbell J A (2000) Intraarterial protein delivery via intimally-adherent bilayer hydrogels. J Controlled Release 64:205-215.
Avens H A, Randle T J, Bowman C N (2008) Polymerization behavior and polymer properties of eosin-mediated surface modification reactions. Polymer 49:4762-4768
Avens H J, Bowman C N (2009) Mechanism of Cyclic Dye Regeneration During Eosin-Sensitized Photoinitiation in the Presence of Polymerization Inhibitors. J Polym Sci Part A: Polym Chemistry 47:6083-6094
Avens H J, Bowman C N (2010) Development of fluorescent polymerization-based amplification for sensitive and non-enzymatic biodetection in antibody microarrays. Acta Biomater 6:83-89
Avens, H. J.; Berron, B. J.; May, A. M.; Voigt, K. R.; Seedorf, G. J.; Balasubramaniam, V. & Bowman, C. N. (2011); Sensitive Immunofluorescent Staining of Cells via Generation of Fluorescent Nanoscale Polymer Films in Response to Biorecognition, Journal of Histochemistry and Cytochemistry 59 (1) 76-87
Avens, H. J.; Chang, E.; May, A. M.; Berron, B. J.; Seedorf, G. J.; Balasubramaniam, V. & Bowman, C. N. (2011), Fluorescent Polymeric Nanocomposite Films Generated by Surface-Mediated Photoinitiation of Polymerization, Journal of Nanoparticle Research 13 (1) 331-346
Avens H J, et al. (2011) J Histochem Cytochem, vol. 59 no. 1, 76-87
Baker C D, Ryan S L, Ingram D A, Seedorf G J, Abman S H, Balasubramaniam V (2009) Endothelial Colony-Forming Cells from Preterm Infants Are Increased and More Susceptible to Hypoxia. Am J Respir Crit. Care Med 180:454-461
Balan L, Schneider R, Lougnot D J (2008) A new convenient route to polyacrylate/silver nanocomposites by light-induced cross-linking polymerization. Prog Org Coat 62:351-357.
Balazs A C, Emrick T, Russell T P (2006) Nanoparticle Polymer Composites: Where
Two Small Worlds Meet. Science 314:1107-1110.
Bowman C N, Kloxin C J (2008) Toward an Enhanced Understanding and Implementation of Photopolymerization Reactions. AIChE J 54:2775-2795.
Bryant S J, Anseth K S (2006) Photopolymerization of Hydrogel Scaffolds. In: Ma P X, Elisseeff J (Eds) Scaffolding in Tissue Engineering. Taylor and Francis Group, Boca Raton, pp 71-90.
Cruise G M, Hegre O D, Scharp D S, Hubbell J A (1998) A Sensitivity Study of the Key Parameters in the Interfacial Photopolymerization of Poly(ethylene glycol) Diacrylate upon Porcine Islets. Biotechnol Bioeng 57:655-665.
Dhibar A K, Mallick S, Rath T, Khatua B B (2009) Effect of Clay Platelet Dispersion as Affected by the Manufacturing Techniques on Thermal and Mechanical Properties of PMMA-Clay Nanocomposites. J Appl Polym Sci 113:3012-3018.
Durán J D G, Arias J L, Gallardo V, Delgado A V (2008) Magnetic Colloids As Drug Vehicles J Pharm Sci 97:2948-2983.
Hansen R R, Sikes H D, Bowman C N (2008a) Visual Detection of Labeled Oligonucleotides Using Visible-Light-Polymerization-Based Amplification. Biomacromolecules 9:355-362
Hansen R R, Avens H J, Shenoy R, Bowman C N (2008b) Quantitative evaluation of oligonucleotide surface concentrations using polymerization-based amplification. Anal Bioanal Chem 392:167-175
Hansen R R, Johnson L M, Bowman C N (2009) Visual, base-specific detection of nucleic acid hybridization using polymerization-based amplification. Anal Biochem 386:285-287
He P, Zheng W, Tucker E Z, Gorman C B, He L (2008) Reversible Addition—Fragmentation Chain Transfer Polymerization in DNA Biosensing. Anal Chem 80:3633-3639
Hunyady B, Mezey E, Pacak K, Palkovits M (1996) Identification of endogenous peroxidase-containing cells as eosinophils in the gastrointestinal system. Histochem Cell Biol 106:447-456
Hutchison J B, Anseth K S (2001) Off-Lattice Approach to simulate Radical Chain Polymerizations of Tetrafunctional Monomers. Macromol Theory Simul 10:600-607.
Ishii K, Muβmann M, MacGregor B J, Amann R (2004) An improved fluorescence in situ hybridization protocol for the identification of bacteria and archaea in marine sediments. FEMS Microbiol Ecol 50:203-212
R. Jenison, H. S. Yang, A. Haeberli, B. Polisky. Interference-based detection of nucleic acid targets on optically coated silicon. Nature Biotechnol. 19 (2001) 62-65.
Jones M Jr (1997) In: Organic Chemistry. WW Norton & Company, New York, pp 238 & 1087.
Kerstens H M J, Poddighe P J, Hanselaar A G J M (1995) A Novel In Situ Hybridization Signal Amplification Method Based on the Deposition of Biotinylated Tyramine. J Histochem Cytochem 43:347-352
Kizilel S, Pérez-Luna V H, Teymour F (2004) Photopolymerization of Poly(Ethylene Glycol) Diacrylate on Eosin-Functionalized Surfaces. Langmuir 20:8652-8658
Kloosterboer J G (1988) Network Formation by Chain Crosslinking Photopolymerization and its Applications in Electronics. Adv Polym Sci 84:1-61.
Kobayashi Y, Misawa K, Kobayashi M, Takeda M, Konno M, Satake M, Kawazoe Y, Ohuchi N, Kasuya A (2004) Silica-coating of fluorescent polystyrene microspheres by a seeded polymerization technique and their photo-bleaching property. Colloids and Surfaces A: Physicochem Eng Aspects 242:47-52.
Lansalot M, Sabor M, Elaissari A, Pichot C (2005) Elaboration of fluorescent and highly magnetic submicronic polymer particles via a stepwise heterocoagulation process. Colloid Polym Sci 283:1267-1277.
Larison K D, BreMiller R, Wells K S, Clements I, Haugland RP (1995) Use of a New Fluorogenic Phosphatase Substrate in Immunohistochemical Applications. J Histochem Cytochem 43:77-83
Lou X, Lewis M S, Gorman C B, He L (2005) Detection of DNA Point Mutation by Atom Transfer Radical Polymerization, Anal Chem 77:4698-4705
Luo Y S, Yang J P, Dai X J, Yang Y, Fu S Y (2009) Preparation and Optical Properties of Novel Transparent Al-Doped-ZnO/Epoxy Nanocomposites. J Phys Chem C 113:9406-9411.
Mayr T, Moser C, Klimant I (2009) Performance of Fluorescent Labels in Sedimentation Bead Arrays—A Comparison Study. J Fluoresc 19:303-310
Olad A, Rashidzadeh A (2008) Preparation and anticorrosive properties of PANI/Na-MMT and PAN I/O-MMT nanocomposites. Prog Org Coat 62:293-298.
Pavlekovic M, Schmid M C, Schmider-Poignee N, Spring S, Pilhofer M, Gaul T, Fiandaca M, Löffler F E, Jetten M, Schleifer K-H, Lee N M (2009) Optimization of three FISH procedures for in situ detection of anaerobic ammonium oxidizing bacteria in biological wastewater treatment. J Microbiol Methods 78:119-126 Popielarski S R, Pun S H, Davis M E (2005) A Nanoparticle-Based Model Delivery System To Guide the Rational Design of Gene Delivery to the Liver. 1. Synthesis and Characterization. Bioconjugate Chem 16:1063-1070.
Satoh M, Shirai K, Saitoh H, Yamauchi T, Tsubokawa N (2005) Photografting of Polymers onto Nanosized Silica Surface Initiated by Eosin Moieties Immobilized onto the Surface. J Polym Sci. Part A: Polym Chem 43:600-606.
Schmidt B F, Choa J, Zhu Z, DeBiasio R L, Fisher G (1997) Signal Amplification in the Detection of Single-copy DNA and RNA by Enzyme-catalyzed Deposition (CARD) of the Novel Fluorescent Reporter Substrate Cy3.29-Tyrmaide. J Histochem Cytochem 45:365-373
Sikes H D, Hansen R R, Johnson L M, Jenison R, Birks J W, Rowlen K L, Bowman C N (2008) Using polymeric materials to generate an amplified response to molecular recognition events. Nat Mat 7:52-56
Sikes H D, Jenison R, Bowman, C N (2009) Antigen detection using polymerization-based amplification. Lab on a Chip 9:653-656
Speel E J M, Hopman A H N, Komminoth P (1999) Amplification Methods to Increase the Sensitivity of In Situ Hybridization: Play CARD(s). J Histochem Cytochem 47:281-288
Van heusden J, de Jong P, Ramaekers F, Bruwiere H, Borgers M, Smets G (1997) Fluorescein-labeled Tyramide Strongly Enhances the Detection of Low Bromodeoxyuridine Incorporation Levels. J Histochem Cytochem 45:315-319
Verma P K, Sardar P S, Ghosh S, Biswas M (2009) Conducting Nanocomposites of Polyacrylamide With Acetylene Black and Polyaniline. Polym Compos 30:490-496.
Wiedorn K H, Kuhl H, Galle J, Caselitz J, Vollmer E (1999) Comparison of in-situ hybridization, direct and indirect in-situ PCR as well as tyramide signal amplification for the detection of HPV. Histochem Cell Biol 111:89-95
Wu X, Liu H, Liu J, Haley K N, Treadway J A, Larson J P, Ge N, Peale F, Bruchez M P (2003) Immunofluorescent labeling of cancer marker Her2 and other cellular targets with semiconductor quantum dots. Nat Biotechnol 21:41-46
Xu X H, Zhang Z Z, Liu W (2009) Fabrication of superhydrophobic surfaces with perfluorooctanoic acid modified TiO₂/polystyrene nanocomposites coating. Colloids Surf: Physiochem Eng Aspects 341:21-26.
Zhu Y, Sun D X, Zheng H, Wei M, Zhang L M (2007) Improvement in properties of epoxy-based electrophoretic coating by silica/polyurethane nanocomposites. J Mater Sci 42:545-550.

We claim:

1. A method of detecting a molecular recognition event between an antibody and a target protein of a cell attached to a substrate, the method comprising the steps of:
a) treating the cell with a blocking agent;
b) contacting the antibody with the target protein under conditions effective to form an antibody-target protein complex;
c) removing antibody not complexed with the target protein;
d) labeling the antibody-target protein complex with a photoinitiator label wherein the photoinitiator label comprises photoinitiator;
e) removing photoinitiator label not attached to the antibody-target protein complex;
f) contacting the photoinitiator-labeled antibody-target protein complex with a polymer precursor solution comprising a water soluble monomer, and a plurality of fluorescent nanoparticles, wherein the average size of the fluorescent particles is from 10 to 50 nm;
g) exposing the photoinitiator-labeled antibody-target protein complex and the polymer precursor solution to light, thereby forming a polymer gel attached to the cell and incorporating a plurality of the fluorescent nanoparticles;
h) removing unpolymerized polymer precursor and fluorescent nanoparticles not incorporated into the polymer gel; and
i) detecting fluorescence from the nanoparticles in the polymer gel, thereby detecting the molecular recognition event.
wherein the ratio of the fluorescence signal per unit area from nanoparticles in the polymer gel to a background fluorescence signal per unit area is greater than 5.

2. The method of claim 1, wherein the target protein is an antigen.

3. The method of claim 1, wherein the cell comprises a plurality of target proteins and a plurality of antibodies are contacted with the cell, forming a plurality of antibody-target protein complexes in step b) and forming polymer gel at a plurality of antibody-target protein complexes in step g).

4. The method of claim 3, wherein the polymer gel forms a layer connected to a plurality of antibody-target protein complexes, the thickness of the layer being from 5 nm to 500 nm.

5. The method of claim 4, wherein the thickness of the layer is from 5 nm to 200 nm.

6. The method of claim 1, wherein the target protein is located in the cell interior and the cell is subjected to a fixation treatment prior to step a).

7. The method of claim 6, further comprising the step of subjecting the cell to a permeabilizing treatment prior to step b).

8. The method of claim 1, wherein the cell is a cultured cell.

9. The method of claim 8, wherein the cell is an adherent cell.

10. The method of claim 1, wherein each of the fluorescent nanoparticles encapsulates a fluorescent dye.

11. The method of claim 10, wherein the surface of the fluorescent nanoparticles is hydrophilic.

12. The method of claim 1, wherein the average size of the fluorescent nanoparticles is from 10 nm to 30 nm.

13. The method of claim 1, wherein the antibody is biotin-labeled, the photoinitiator label further comprises a biotin-binding protein and the antibody-target protein complex is labeled with the photoinitiator label by interaction between the biotin of the antibody and the biotin-binding protein of the photoinitiator label.

14. The method of claim 1 wherein the antibody-target protein complex is a primary antibody-target protein complex and the primary antibody-target protein complex is labeled with the photoinitiator label by contacting the antibody target protein-complex with a secondary antibody bound to a photoinitiator label, the secondary antibody undergoing molecular recognition with the primary antibody.

15. The method of claim 1, wherein the antibody-target protein I complex is a primary antibody-target protein complex and the primary antibody-target protein complex is labeled with the photoinitiator label by contacting the primary antibody target protein-complex with a secondary antibody comprising biotin and undergoing molecular recognition with the primary antibody, thereby forming a biotin-labeled complex of target protein with the primary and secondary antibodies and then contacting the biotin-labeled complex of target protein with the primary and secondary antibodies with a photoinitiator label comprising a biotin-binding protein.

16. The method of claim 1, wherein the photoinitiator is a photoreducible dye, the polymer precursor solution further comprises a co-initiator and the photoinitiator-labeled antibody-target protein complex and the polymer precursor solution are exposed to visible light in step g).

17. The method of claim 1, wherein the water soluble monomer comprises at least two acrylate or acrylate derivative functional groups.

18. The method of claim 17, wherein the polymer precursor solution further includes a second water soluble monomer comprising a single acrylate or acrylate derivative functional group.

19. The method of claim 18, wherein the amount of the first monomer relative to the amount of the first and second monomers is from 50% to less than 100%.

20. A kit comprising:
a) a first aqueous solution including a conjugate of a photoreducible dye photoinitiator and a secondary antibody;
b) a second aqueous solution including a water soluble monomer, an amine co-initiator and a polymerization accelerator; and
c) a third aqueous solution including fluorescent nanoparticles.

21. A kit comprising:
a) a first aqueous solution including a conjugate of a photoreducible dye photoinitiator and biotin-binding protein;
b) a second aqueous solution including a biotinylated secondary antibody;
c) a third aqueous solution including a water soluble monomer, an amine co-initiator and a polymerization accelerator; and
d) a fourth aqueous solution including fluorescent nanoparticles.

22. A kit comprising:
a) a first aqueous solution including a conjugate of a photocleavable photoinitiator and biotin-binding protein;
b) a second aqueous solution including a biotinylated secondary antibody;
c) a third aqueous solution including a water soluble monomer and a polymerization accelerator; and
d) a fourth aqueous solution including fluorescent nanoparticles.
